(19) United States
(12) Patent Application Publication
(10) Pub. No.: US 2010/0207078 A1
(43) Pub. Date: Aug. 19, 2010

(54) DEPROTECTION OF FUNCTIONAL GROUPS BY MULTI-PHOTON INDUCED ELECTRON TRANSFER

(76) Inventors: Seth Marder, Atlanta, GA (US); Stephen Barlow, Atlanta, GA (US); Joseph Perry, Sandy Springs, GA (US); Jing Wang, Tucson, AZ (US)

Correspondence Address: MERCHANT & GOULD PC, P.O. BOX 2903, MINNEAPOLIS, MN, 55402-0903, US

(21) Appl. No.: 12/373,318

(22) PCT Filed: Jul. 12, 2007
(86) PCT No.: PCT/US07/15971

§ 371 Date: Sep. 22, 2009

Related U.S. Application Data
(60) Provisional application No. 60/830,159, filed on Jul. 12, 2006.

(51) Int. Cl. G02B 5/23; C07C 69/76; C07C 69/612

(52) U.S. Cl. 252/586; 560/54; 560/51; 560/251; 204/157.69; 204/157.6

(57) ABSTRACT

The present invention provides novel compositions suitable for use in an intermolecular photodeprotection reaction scheme. Such compositions include a chromophore compound and a second compound having a photocleavable group bonded to a protected functional group. Novel compounds which can used in intramolecular photodeprotection are also provided. These compounds have a chromophore moiety bonded to a photocleavable group, which itself is bonded to a protected group. The compounds and compositions disclosed herein can be used in two-photon and multi-photon excitation.

This application is being filed on Jul. 12, 2007, as a PCT International Patent application in the name of Georgia Tech Research Corporation and The Arizona Board of Regents on Behalf of The University of Arizona, both U.S. national corporations, applicant for the designation of all countries except the US, and Seth Marder a citizen of the United States; Stephen Barlow, a citizen of the United Kingdom; Joe Perry and citizen of the United States; and Jing Wang a citizen of China; applicants for the designation of the US only, and claims priority to U.S. Provisional patent application Ser. No. 60/830,159, filed Jul. 12, 2006.

### BACKGROUND OF THE INVENTION

The ability to protect a reactive functional group and provide that group in stable form, yet deliver the reactive functionality upon subjecting the protected group to deprotection conditions, constitutes a useful synthetic and mechanistic tool. Deprotection processes can be carried in any number of ways, including thermally or photochemically. The present invention relates generally to the absorption of radiation by a chromophore moiety or compound, resulting in electron transfer and subsequent cleavage of a protected functional group from a photocleavable group.

Chromophore moieties or compounds that can absorb electromagnetic radiation can play a role in the process of protection and deprotection. Chromophores can encompass many different chemical structures. Various components observed in organic chromophores include conjugated systems, aromatic systems, and donor or acceptor moieties. A more complete description of electron donors or donating groups and electron acceptors or electron accepting groups is disclosed in J. March, Advanced Organic Chemistry Reactions, Mechanisms and Structure, Fourth edition, Wiley-Interscience, New York, 1992. Chapter 9.

Thus, there is a continuing need to develop and understand new applications for chromophore moieties and to incorporate such moieties into new reactions and processes. There is also a need to develop new and improved methods to initiate deprotection, and new methods for inducing electron transfer and subsequent cleavage of a protected functional group from a photocleavable group.

### BRIEF SUMMARY OF THE INVENTION

In one aspect, this invention provides a system in which two-photon absorption leads to an excited state, which is capable of transferring an electron to an acceptor group which then undergoes cleavage to release a protected functionality. The general strategy comprises a two-photon absorbing chromophore which transfers an electron to a group that we will refer to as the photocleavable group. The photocleavable group will be chosen such that after receiving an electron it will undergo a bond cleavage process to release the protected group of interest. Specifically, by two-photon or multi-photon excitation, we refer to a method of preparing a compound in an electronically excited state, comprising the steps of:

(a) exposing a compound to pulsed laser radiation; and

(b) converting the compound to a multiphoton electronically excited state upon simultaneous absorption of at least two photons of radiation by the compound, wherein the sum of the energies of all of the absorbed photons is greater than or equal to the transition energy from a ground state of the compound to the multi-photon excited state and wherein the energy of each absorbed photon is less than the transition energy between the ground state and the lowest single-photon excited state of the compound and is less than the transition energy between the multi-photon excited state and the ground state.

The photodeprotection method of the present invention can be carried out in an intermolecular or an intramolecular fashion. For example, an intermolecular photodeprotection can be carried out in which at least one chromophore compound is excited and electrons are transferred from the chromophore compound to a second compound having a photocleavable group bonded to a protected group. In another aspect, this invention encompasses an intramolecular photodeprotection, in which a single compound comprising a chromophore moiety bonded to a photocleavable group, which itself is bonded to a protected group.

It has been discovered that molecules that have two or more electron donors such as amino groups or alkoxy groups connected to aromatic or heteroaromatic groups as part of a π(pi)-electron bridge exhibit unexpectedly and unusually high two-photon or higher-order absorptivities. These high two-photon or higher-order absorptivities are compared to, for example, the absorptivities observed in, for example, dyes, such as stilbene, diphenylpolyenes, phenylene vinylene oligomers and related molecules.

Moreover, it has also been discovered that the strength and position of the two-photon or higher-order absorption can be tuned and further enhanced by appropriate substitution of the π-electron bridge with accepting groups such as cyano. It was also discovered in accordance with the present invention that molecules that have two or more electron acceptors such as formyl or dicyanomethylidene groups connected to aromatic or heteroaromatic groups as part of a π(pi)-electron bridge, exhibit unexpectedly and unusually high two-photon or higher order absorptivities in comparison to, for example, dyes, such as those disclosed above. Further, it has also been observed that the strength and position of the two-photon or higher-order absorption can be tuned and further enhanced by appropriate substitution of the n-electron bridge with donating groups such as methoxy.

Compounds and compositions of the present invention are useful when incorporated into solutions, prepolymers, polymers, Langmuir-Blodgett thin films, and self-assembled mono-layers. Such compounds and compositions can be modified in such a way as to allow for variation of ease of dissolution in a variety of host media, including liquids and polymeric hosts, by changing the nature of the substituents attached to the central pi-conjugated framework of the molecule as well as either the donors or acceptors. In addition, by controlling the length and composition of the π-bridge of the molecule, it is possible to control the position and strength of the two-photon or higher-order absorption and the two-photon or higher-order excited fluorescence.

### BRIEF DESCRIPTION OF THE DRAWINGS

FIG. 1 is a flow diagram illustrating a photodeprotection scheme of the present invention.

FIG. 2 illustrates an energy level diagram for the photodeprotection via electron transfer mechanism from a two-photon-excited chromophore.

FIG. 3 is a plot of absorbance versus wavelength for Compound (10).

FIG. 4 is a plot of photoluminescence versus wavelength for Compound (10).

FIG. 5 is a plot of absorbance versus wavelength for Compound (16).

FIG. 6 is a plot of photoluminescence versus wavelength for Compound (16).

FIG. 7 is a plot of photoluminescence versus wavelength for Compound (10) in acetonitrile.

FIG. 8 is a plot of integrated fluorescence versus absorption at 420 nm for Compound (10) in acetonitrile.

FIG. 9 is a plot of photoluminescence versus wavelength for Compound (16) in acetonitrile.

FIG. 10 is a plot of integrated fluorescence versus absorption at 420 nm for Compound (16) in acetonitrile.

FIG. 11 is a cyclic voltammetry plot for Compound (1).

FIG. 12 is a cyclic voltammetry plot for the compound in Reaction Scheme A with R═C₆H₅p-CH₃.

FIG. 13 is a positive cyclic voltammetry plot for Compound (5).

FIG. 14 is a negative cyclic voltammetry plot for Compound (5).

FIG. 15 is a cyclic voltammetry plot for Compound (13).

FIG. 16 is a cyclic voltammetry plot for Compound (18).

FIG. 17 is a positive cyclic voltammetry plot for Compound (18).

FIG. 18 is a cyclic voltammetry plot for Compound (23).

FIG. 19 is a positive cyclic voltammetry plot for Compound (23).

FIG. 20 is an approximate energy level diagram constructed using optical data and electrochemical data for two-photon chromophores and photocleavable groups.

FIG. 21 illustrates the progress of the photodeprotection reaction scheme of Example 12.

FIG. 22 illustrates the progress of the photodeprotection reaction scheme of Example 13.

FIG. 23 illustrates the progress of the photodeprotection reaction scheme of Example 14.

### ABBREVIATIONS

The following abbreviations are provided in order to aid those skilled in the art in understanding the detailed description of the present invention.

ACN—acetonitrile

AcOH—acetic acid

app—appeared

BtOH—tertiary-butyl alcohol

Bu—butyl

^{t}Bu—tert-butyl

BuOH—butyl alcohol

(CH₂O)ₙ—paraformaldehyde

CYH—cyclohexane

DBU—1,8-diazabicyclo[5,4,0]-undec-7-ene

DCC—N,N′-dicyclohexylcarbodimine

DCM—dichloromethane

DIAD—diisopropyl azocarboxylate

dis—disappeared

DMAP—4-dimethylaminopyridine

DMF—N,N-dimethylforamide

DMSO—dimethyl sulfoxide

Et or ET—ethyl

ETH—ethanol

ΔG_{ET}—Gibbs free energy change associated with electron transfer

h—hour(s)

k_{BET}—rate constant for back-electron transfer

k_{cleav}—rate constant for cleavage

k_{ET}—rate constant for electron transfer

k_{non-rad}—rate constant for non-radiative decay

k_{rad}—rate constant for radiative decay

Me—methyl

MS—mass spectrometry

OAc—acetate

rt—room temperature

THF—tetrahydrofuran

TLC—thin layer chromatography

TOL—toluene

### DETAILED DESCRIPTION OF THE INVENTION

### Two-Photon or Multi-Photon Deprotection

This invention employs a novel method in which two-photon, or multi-photon, absorption leads to an excited state in a compound or moiety, which is capable of transferring an electron to an acceptor group, which then undergoes cleavage to release a protected functionality. In this disclosure, wherever two-photon is used to describe excitation or absorption, multi-photon (more than two) can also be employed.

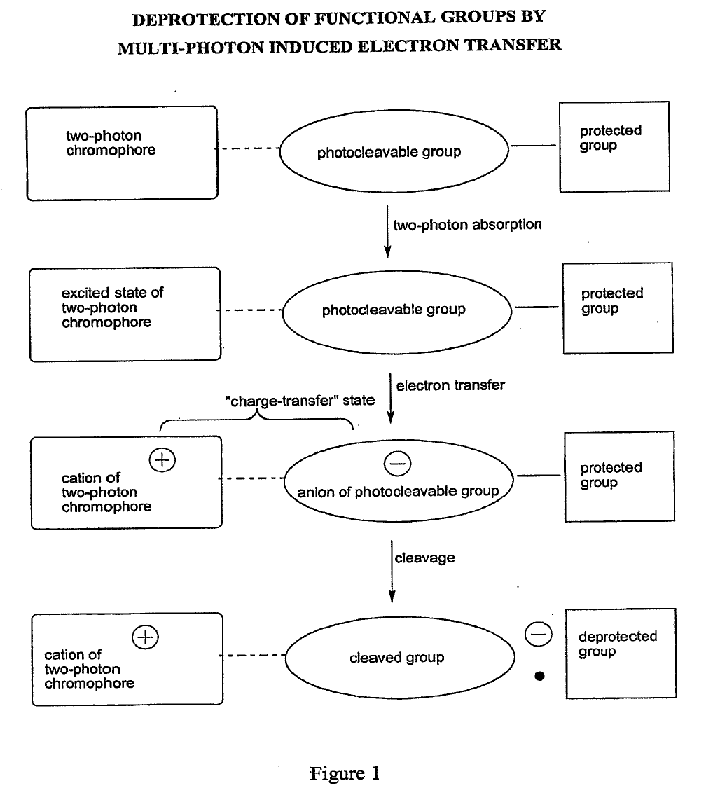

FIG. 1 illustrates a generic scheme showing two-photon photodeprotection via electron transfer. The general strategy comprises a two-photon absorbing chromophore, which transfers an electron to a group referred to as a photocleavable group. The photocleavable group is designed such that after receiving an electron it will undergo a bond cleavage process to release the protected functional group of interest. The final deprotected product can be released as an anion or a radical depending on the photocleavable group used. The dotted line indicates the possibility of covalent bonding, non-covalent interactions, or no interaction between the chromophore and the photocleavable group. Hence, both intramolecular and intermolecular photodeprotection are within the scope of this invention. The positive and negative charges shown only indicate the change of charge relative to the original system, where any of the groups may be either neutral, positively charged, or negatively charged.

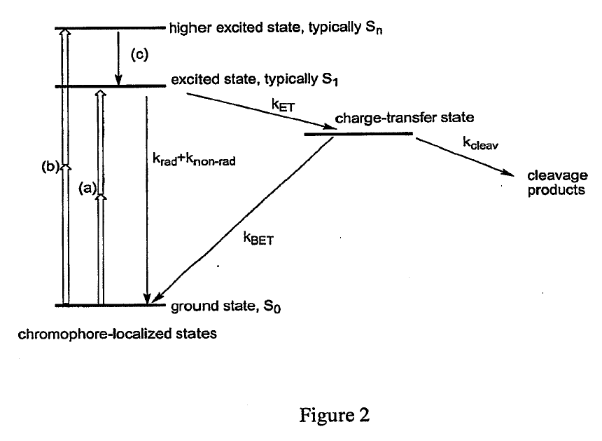

Two-photon excitation of a chromophore is the process whereby two photons of equal or unequal energy are absorbed to create an excited state whose energy, relative to the ground state, is the sum of the energy of the two photons. The electron transfer may occur directly from this excited state or from another state to which the two-photon state is converted by internal conversion. FIG. 2 illustrates the possibility of two-photon absorption (with photons of the same energy) either (a) into the state from which electron transfer takes place, typically the first singlet excited state S₁, or (b) into a higher-lying singlet state, Sₙ, followed by (c) internal conversion, allowing relaxation to the state from which electron transfer can take place.

While not wishing to be bound by theory, Applicants believe that several criteria are associated with an effective two-photon photodeprotection system. These include:

(1) The two-photon absorption cross-section into one or more excited states, corresponding to absorption of photons of energy lower than that for the lowest one-photon electronic transition of the chromophore, has a magnitude of at least about 50×10⁻⁵⁰ cm⁴s/photon. In another aspect of this invention, the chromophore moiety or compound of the present invention has a two-photon absorption cross-section of greater than about 75×10⁻⁵⁰ cm⁴s/photon, or greater than about 100×10⁻⁵⁰ cm⁴s/photon. Yet, in another aspect, the chromophore moiety or compound has a two-photon absorption cross-section of greater than about 200×10⁻⁵⁰ cm⁴s/photon, greater than about 300×10⁻⁵⁰ cm⁴s/photon, or greater than about 400×10⁻⁵⁰ cm⁴s/photon. In accordance with the present invention, the chromophore moiety or compound can have a two-photon absorption cross-section of greater than about 500×10⁻⁵⁰ cm⁴s/photon.

(2) The electron transfer from the final state obtained on relaxation of the two-photon excited state of the chromophore moiety or compound to the photocleavable group is thermodynamically feasible, i.e., the charge-transfer state should be lower in energy than the relaxed state obtained on relaxation of the chromophore moiety or compound, or the Gibbs free energy change, ΔG_{ET}, associated with electron transfer should be negative. The feasibility of electron transfer can be estimated from:

ΔG_{ET}=FE_{1/2}(C^{+/}*)−FE_{1/2}(P^{0/-}),

where F is the Faraday constant, E_{1/2}(P^{0/-}) is the half wave potential corresponding to the process, e⁻+P→P⁻, where P is the photocleavable group and this can be measured or estimated from electrochemical data, and E_{1/2}(C^{+/}*) is the electrochemical half wave potential corresponding to the process, e⁻+C⁺→C*, where C⁺ and C*, respectively, represent the chromophore minus one electron and the excited state of the chromophore formed by excitation and relaxation (typically excited state S₁). This potential can, in turn, be estimated according to:

E_{1/2}(C^{+/}*)=E_{1/2}(C^{+/0})−[E₀₀/F],

where E_{1/2}(C^{+/0}) is the electrochemically measured or estimated value of the half-wave potential corresponding to the process, e⁻+C⁺→C, where F is the Faraday constant and E₀₀ is the energy of the relaxed excited state above that of the ground state, which can generally be estimated as the energy at which the one-photon absorption onset of the chromophore is seen or at which normalized fluorescence and one-photon absorption spectra intersect (for chromophores wherein the lowest excited state is one-photon allowed and which obey Kasha's rule).

Thus, in one aspect of the present invention, the Gibbs free energy change associated with electron transfer from the chromophore moiety or chromophore compound to the photocleavable group (ΔG_{ET}) is less than +28 kJ/mol. In another aspect, ΔG_{ET} is less than +21 kJ/mol, or less than +14 kJ/mol. In a different aspect, the Gibbs free energy change associated with electron transfer from the chromophore moiety or chromophore compound to the photocleavable group (ΔG_{ET}) is less than zero kJ/mol.

(3) The rate of forward electron transfer should be competitive with other processes that can depopulate the chromophore-based excited state. That is, k_{ET}>0.1(k_{rad} plus k_{non-rad}), where k_{ET}, k_{rad}, and k_{non-rad} are rate constants, respectively, for the electron transfer, radiative decay, and non-radiative decay of the relevant chromophore-based excited state. Rates of intermolecular electron transfer are known to those skilled in the art to depend on the thermodynamic driving force for electron transfer and on the electronic coupling between the donor and acceptor moieties. The driving force will depend on the choice of donor and acceptor functionalities, whereas the electronic coupling will depend on the specific details of the linkage between donor and acceptor (the dotted line illustrated in FIG. 1).

Hence, in one aspect of the present invention, the rate constant for electron transfer, k_{ET}, of an excited state of the chromophore moiety is greater than 0.1×(rate constant for radiative decay plus the rate constant for non-radiative decay) (k_{rad}+k_{non-rad}) of the excited state of the chromophore moiety. In another aspect, the rate constant for electron transfer, k_{ET}, is greater than 0.5×(k_{rad}+k_{non-rad}). Alternatively, the rate constant for electron transfer, k_{ET}, is greater than the sum of k_{rad} and k_{non-rad} in other aspects of the present invention.

(4) Cleavage should be thermodynamically feasible from the charge-transfer state, i.e., from the photocleavable group plus one electron.

(5) The rate of cleavage of a charge-transfer state, i.e., of the photocleavable group plus one electron, should be competitive with processes depopulating the charge-transfer state. In the present invention, the rate constant for cleavage, k_{cleav}, of a charge-transfer state of the photocleavable group is greater than 0.1 times the rate constant for back-electron transfer, k_{BET}, of the charge-transfer state of the photocleavable group. In another aspect, k_{cleav} is greater than 0.5 times k_{BET}. In still another aspect, k_{cleav} is greater than k_{BET}, greater than 2 times k_{BET}, greater than 3 times k_{BET}, or greater than 4 times k_{BET}. In a further aspect, the rate constant for cleavage, k_{cleav}, of a charge-transfer state of the photocleavable group is greater than 5 times the rate constant for back-electron transfer, K_{BET}, of the charge-transfer state of the photocleavable group.

It is contemplated in other aspects of the present invention that the chromophore moieties or chromophore compounds disclosed herein have two-photon absorption cross-sections in the wavelength range between 300 nm and 1900 nm. In some aspects, the chromophore moieties or chromophore compounds have two-photon absorption cross-sections in the wavelength range between 400 nm and 800 nm. In others aspect, the wavelength range is between 350 nm and 600 nm, 500 nm and 700 nm, or 600 nm and 900 nm.

Another feature of the invention is that the oxidation potential, reduction potential and the energy difference between the ground state and the fluorescent excited state can be precisely tuned such that the excited-state reduction or oxidation potential can also be tuned. In this manner, using the theory for electron transfer developed by Marcus, it is possible to tune both the forward electron transfer rate and charge recombination rate. This tunability allows control of, for example, the initiation rate of the polymerization, or the time constants for the generation and recovery in transient photochromic materials.

Many Of the compounds useful according to the invention can be described by one of four structural motifs. These compounds exhibit enhanced two-photon or multi-photon absorptivities and allow one to control the position of two-photon or multi-photon absorption bands. The motifs may be generally categorized as follows:

(a) molecules in which two donors are connected to a conjugated π-electron bridge (abbreviated “D-π-D” motif);

(b) molecules in which two donors are connected to a conjugated π-electron bridge which is substituted with one or more electron accepting groups (abbreviated “D-A-D” motif);

(c) molecules in which two acceptors are connected to a conjugated π-electron bridge (abbreviated “A-π-A” motif); and

(d) molecules in which two acceptors are connected to a conjugated π-electron bridge which is substituted with one or more electron donating groups (abbreviated “A-D-A” motif).

Yet another feature of this invention is that it is possible to control the position of the two-photon or higher order absorption peak in these molecules by controlling by the number of conjugated double bonds between the two donor substituted aromatic or heteroaromatic for class one compounds, or the two electron acceptor substituted aromatic or heteroaromatic end groups for class two compounds. Class one compounds are compounds where the end groups are electron donating groups, e.g., D-π-D and D-A-D. Class two compounds are compounds where the end groups are acceptors, e.g., A-π-A and A-D-A. Thus, increasing the number of double bonds, also leads to a considerable shift of the two-photon or higher order absorption band to longer wavelength. Incorporation of phenylene-vinylene groups between the end groups has a similar effect.

The lipophilicity, hydrophilicity and overall solubility of the two-photon or higher absorbing chromophores can be tuned over a very wide range by the appropriate substitution of the donor groups. For example, 4,4′-bis-dimethylaminostilbene and 4,4′-bis-(diphenyl)aminostilbene are sparingly soluble, whereas 4,4′-bis(-di-n-butylamino)stilbene and 4,4′-bis(di-(4-n-butyl-phenyl)aminostilbene are very soluble in non-polar organic solvents, i.e., are lipophilic. In contrast, the hydrochloric acid adduct of the bis-lysyl ester of 4-diethylamino 4′-diethanolamino stilbene and bis-lysyl ester of 1-(4-dimethylaminophenyl)-4-(4′-diethanolaminophenyl)buta-1,3-diene are hydrophilic. In each case, the molecules maintain their fluorescence in solution.

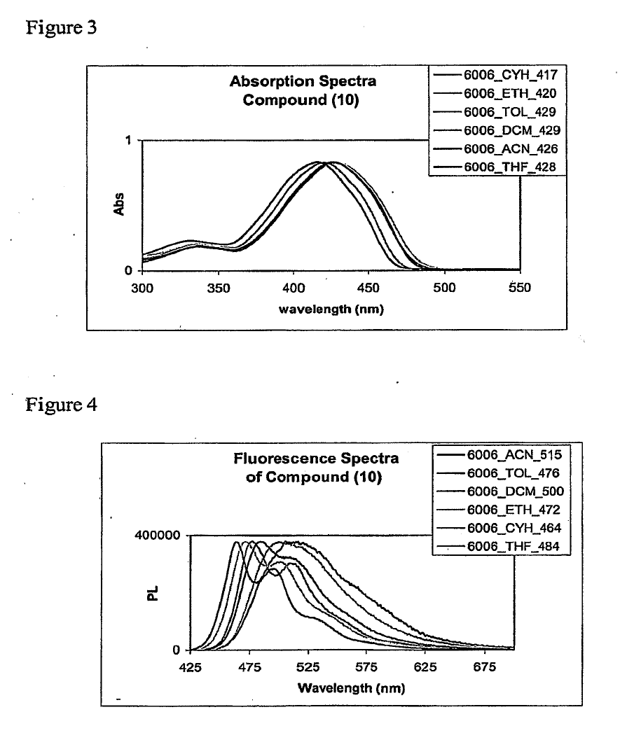

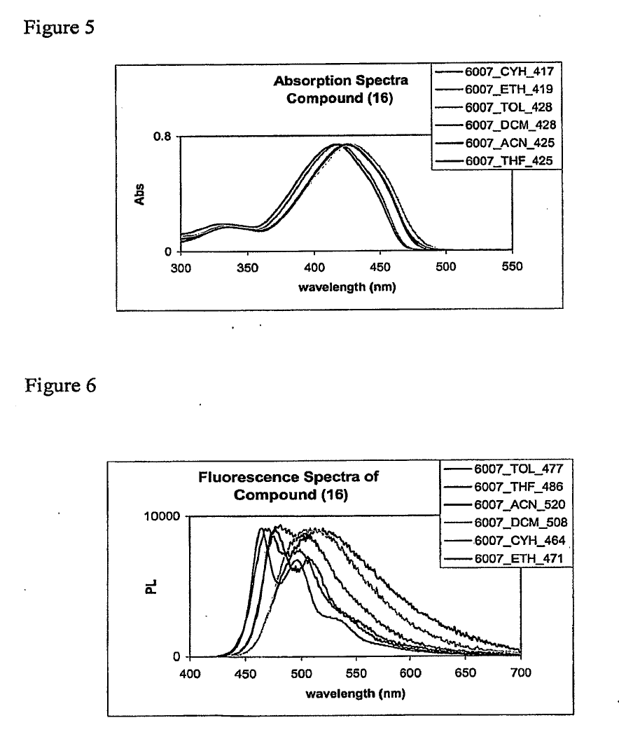

FIGS. 3 and 4 illustrate the absorption and fluorescence spectra, respectively, for Compound (10). Similarly, FIGS. 5 and 6 illustrate the absorption and fluorescence spectra, respectively, for Compound (16). Absorption spectra were determined on a Hewlett-Packard model 8453 spectrophotometer. Fluorescence spectra were collected on a Jobin Yvon Spex Fluorolog-III fluorimeter. Photon parameters were measured by using a multi-functional optical meter of Newport model 1835-C. The data in FIGS. 3-6 were determined using one photon excitations. Compound (10) is synthesized in Example 4 and is a chromophore compound. Compound (16) is synthesized in Example 7 and is a compound containing a chromophore moiety analogous to Compound (10) bonded to a photocleavable group. A comparison of the absorption spectra indicates that the addition of a photocleavable group in Compound (16) does not impact the one-photon absorption. Thus, a skilled artisan would also expect no impact due to the presence of the photocleavable group on the absorption of the chromophore moiety in a two-photon absorbance as well, albeit that such absorption spectra typically would be conducted in the 500 nm to 900 nm range, for example.

A comparison of the one-photon fluorescence spectra in FIGS. 4 and 6 indicates an approximate 40-fold drop in the fluorescence of Compound (16), containing a photocleavable group, versus that of Compound (10). This drop illustrates a reduction in fluorescence of Compound (16) due to electron transfer competing with fluorescence in Compound (16). All of the chromophore molecules or chromophore moieties of this disclosure are two-photon excitable, which leads to the same excited state following non-radiative decay as obtained in the single photon excitations. Accordingly, cleavage of a protected functional group from a photocleavable group, arising from the absorption by the chromophore and electron transfer, operate identically regardless of whether the excited state is generated via single photon or multi photon excitations.

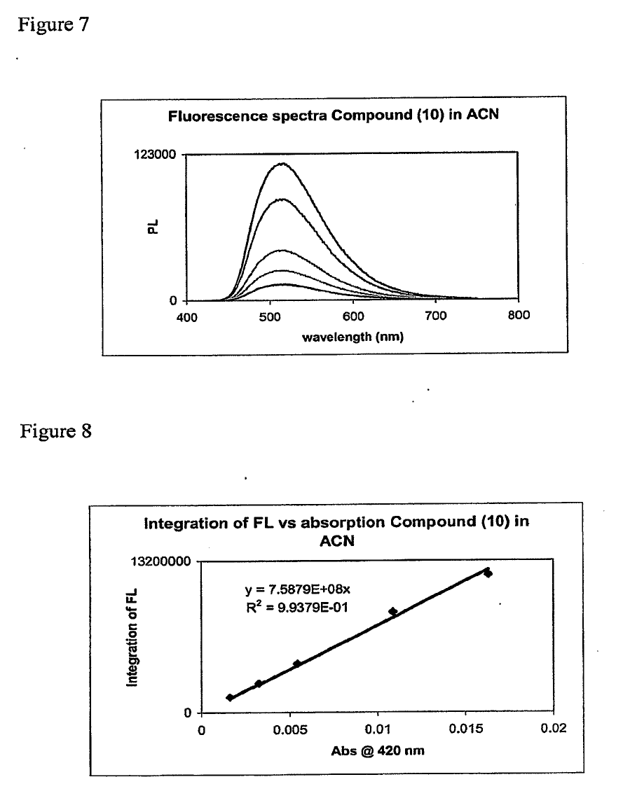

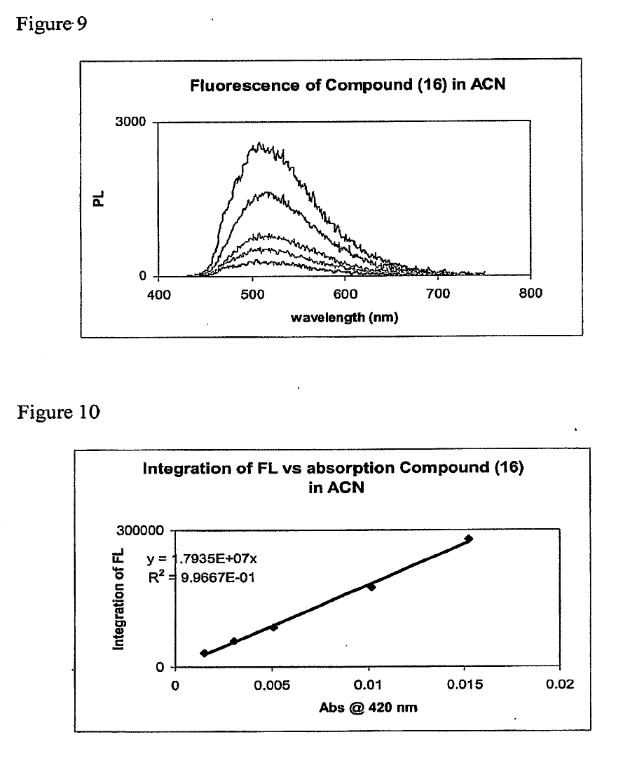

This electron transfer process is further evidenced by comparing FIGS. 7 and 9, which show that one-photon fluorescence quantum yield of Compound (16) is about 2.4% of that of Compound (10), due to fluorescence quenching by electron transfer being competitive with the radiative and nonradiative rates for depopulation of the excited state of the molecules. This provides support for molecular design consistent with the photodeprotection factors describe above. FIGS. 8 and 10 demonstrate a linear relationship between the integrated fluorescence and absorption, regardless of the presence of a photocleavable group. The linear behavior of the integration of fluorescence vs. absorption shown in FIGS. 8 and 10 is useful for making accurate quantum yield calculations.

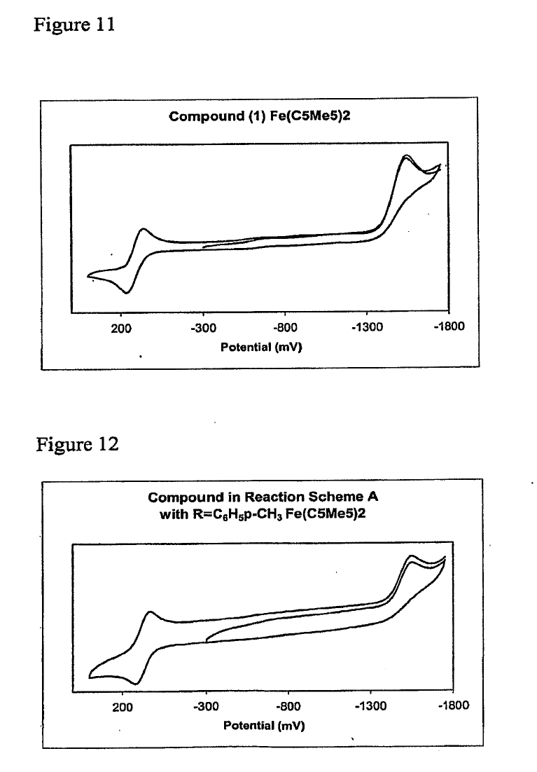

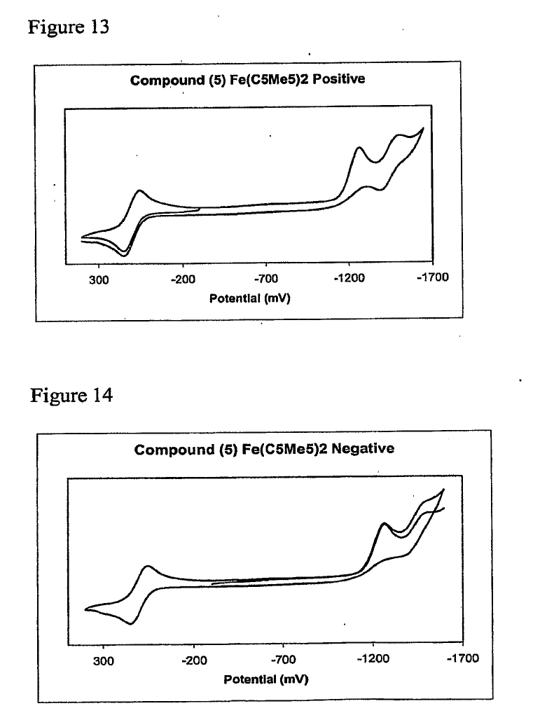

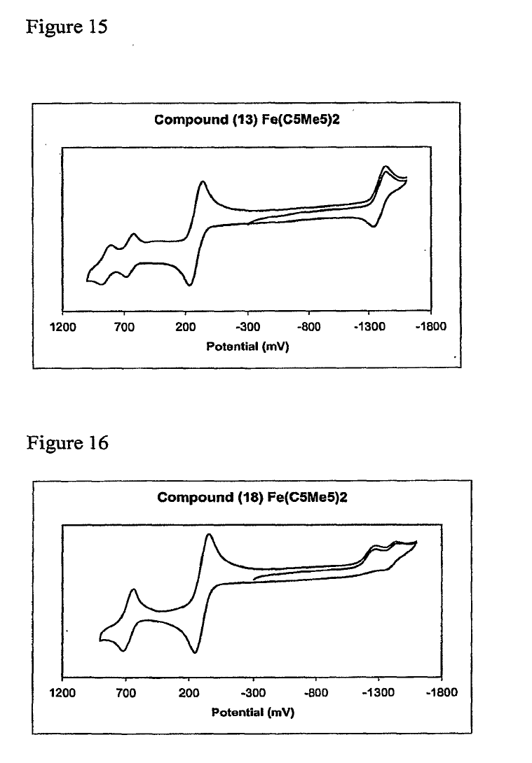

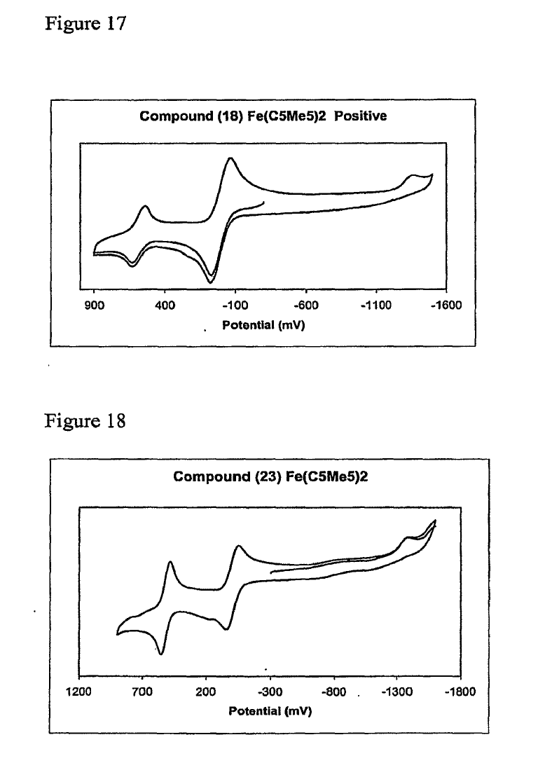

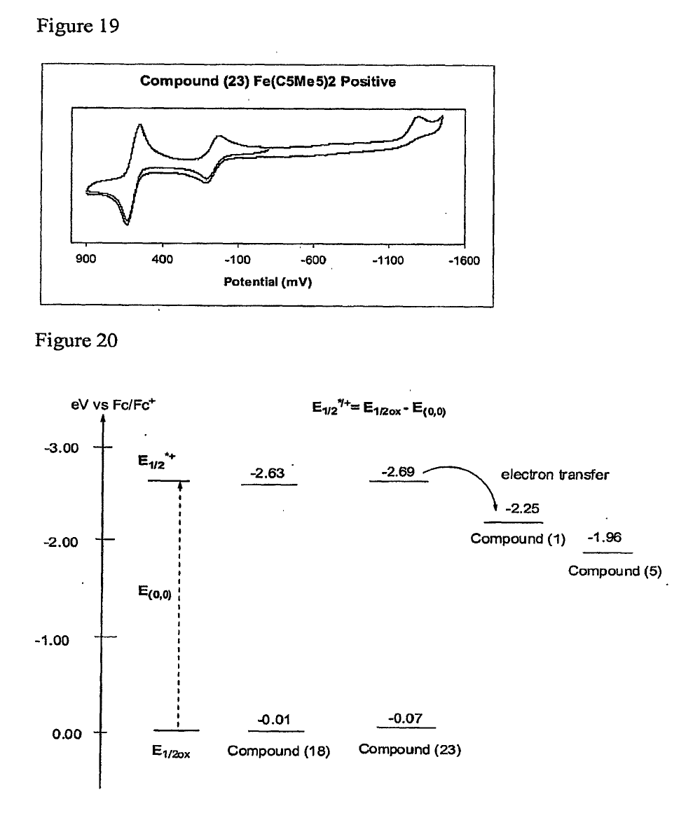

Cyclic voltammograms show redox processes of the compound indicated in addition to that of decamethylferrocene internal reference (−0.48 V vs. ferrocenium/ferrocene in CH₂Cl₂: Connelly and Geiger, Chem. Rev. 1996, vol. 96, p 877 and seen at +0.1-0.2 V vs. the pseudo-reference electrode used for horizontal scale). FIGS. 11-19 are cyclic voltammetry plots that demonstrate that the energy of the excited state of the chromophore or the oxidation potential of the chromophore is sufficient to reduce the photocleavable group via electron transfer (i.e., cleavage is possible). FIG. 20 further demonstrates that the energy level of the excited state of the chromophore provides the necessary driving force for electron transfer. Thus, the cyclic voltammetry measurements are useful for measuring the oxidation potential of the chromophore, which can be added to the energy of the photon to obtain the reduction potential of the first excited state, which addresses whether the first excited state is sufficient to cleave the photocleavable group.

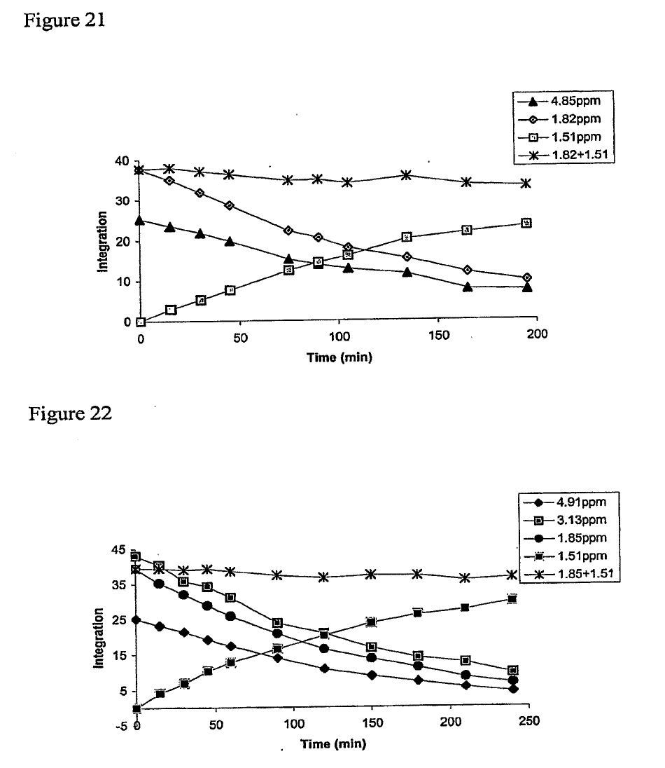

Examples 12-14 and FIGS. 21-23 demonstrate that both intramolecular and intermolecular reaction schemes are successful in cleaving and deprotecting a protected functional group using one-photon excitation. Example 11 demonstrates an unsuccessful photodeprotection scheme; excitation of the chromophore was not enough to cleave and deprotecting the acetic acid functional group of Compound (5). Constructive Examples 15-16 demonstrate intermolecular and intermolecular photodeprotection schemes using two-photon excitation.

These data demonstrate that both intermolecular and intramolecular systems composed of a two-photon chromophore and a photocleavable group can be cleaved by excitation with light of a wavelength absorbed by the two-photon chromophore, but not directly absorbed by the photocleavable group. The electrochemical data described above indicate the thermodynamic favorability of electron transfer from the excited chromophore to the photocleavable group in all cases. Example 11 shows that the protected group of Compound (5) is not cleaved, although electron transfer is favorable. In this case, we teach that the second compound does not undergo cleavage sufficiently effectively for cleavage to occur before the competitive back electron transfer. In the case of Examples 12-14, cleavage is successful due to the selection of the photocleavable group in the second compound. It is known that the chromophores shown in these examples have two-photon cross-sections, either by measurement or by analogy to closely related structures/compounds that have been measured. For example, Compound (11), also known as dye 41, has a cross-section of about 900×10⁻⁵⁰ cm⁴s/photon at 730 nm wavelength, as disclosed in Rumi et al., J. Am. Chem. Soc., 2000, 122, 9500-9510. Therefore, excitation of these systems with intense light of about 730 nm can be anticipated to lead to two-photon excitation followed by electron transfer and cleavage analogous to that described herein by one-photon excitation.

U.S. Pat. No. 6,267,913 discloses a method for multi-photon deprotection or photodecaging of groups in which a multi-photon absorbing dye is attached to known photodeprotecting groups or photodecaging group. In this method, a dye can absorb two-photons or more of energy, and through an energy or charge transfer mechanism, serve to excite the attached photodeprotecting or photodecaging group, thereby inducing the deprotection of a functional group. The functional group can be, for example, a drug, neurotransmitter, metal ion or other chemical reagent. In contrast, the present invention discloses that it is not necessary to “excite” the attached photodeprotecting group, but rather, effective cleavage depends largely upon other criteria. One such criteria is that the excited chromophore transfers an electron to the photocleavable group.

Further, in the present invention, the rate of forward electron-transfer should be competitive with other processes that can depopulate the chromophore-based excited state. That is, k_{ET}>0.1 times (k_{rad} plus k_{non-rad}), where k_{ET}, k_{rad} and k_{non-rad} are rate constants; respectively, for the electron transfer, and for the radiative and non-radiative decay of the relevant chromophore-based excited state. As noted earlier, rates of intermolecular electron transfer are known to those skilled in the art to depend on the thermodynamic driving force for electron transfer and on the electronic coupling between the donor and acceptor moieties. Additionally, the driving force will depend on the choice of donor and acceptor functionalities, whereas the electronic coupling will depend on the details of the linkage between donor and acceptor.

Also, cleavage should be thermodynamically feasible from the charge-transfer state, i.e., from the photocleavable group plus one electron. For instance, the rate of cleavage of the charge-transfer state, i.e., of the photocleavable group plus one electron, should be competitive with processes depopulating the charge transfer state. That is, k_{cleav}>0.1 times k_{BET}, where k_{cleav} and k_{BET} are rate constants for cleavage and for back-electron transfer.

As shown in Examples 11-13, it can be seen that in one case (Example 11) electron transfer does not lead to effective cleavage, whereas in Examples 12-13 the rate of cleavage is competitive with back reaction and the desired photodeprotection reaction occurs. While not intending to be bound by theory, it is conceivable that because Compound (5) in Example 11 has two carbonyl groups, the rate of forward electron transfer may be more energetically favorable as compared to that for the compounds of Examples 12-13. In the latter examples, the radical anion can be localized more on the carbonyl adjacent to the carbon involved in the cleavage reaction, which facilitates the subsequent cleavage reaction. Thus, the present invention teaches that excitation of or transference of an electron to the photocleavable group, alone, is not sufficient to ensure cleavage and that all the criteria presented above are necessary to distinguish operative compounds and compositions of this invention that are useful in intramolecular and intermolecular photodeprotection from those disclosed elsewhere.

Moreover, the same strategy will be applicable for any two-photon chromophore, with two-photon absorption at any wavelength, any cleavable group, and any protected functionality, as long as the criteria discussed above are met. Further details of possible choices of chromophore, cleavable group, and the protected group are given below. This invention also demonstrates that photodeprotection can be carried out either in an intermolecular or intramolecular fashion. Intramolecular photodeprotection, in some instances, can be employed at lower concentrations than intermolecular compositions. Generally, any type of covalent link may be selected in the intramolecular case, as may non-covalent links between the chromophore moiety and photocleavable moiety. This may include, but is not limited to, hydrogen bonding, π-stacking, arene-perfluoroarene interactions, and ionic other electrostatic interactions. The choice of linking mechanism may be anticipated to affect both forward and back electron transfer rates and so will affect the rates of cleavage obtainable under a given irradiation.

### Intermolecular Photodeprotection

A composition in accordance with the present invention can be used in an intermolecular photodeprotection scheme whereby at least one chromophore compound is excited by light or other radiation source and electrons are transferred from the chromophore compound to at least one second compound. The at least one second compound has at least one photocleavable group bonded to at least one protected group. The electron transfer causes the protected functional group to be cleaved from the at least one second compound.

In one aspect, the present invention provides a composition comprising:

(a) at least one chromophore compound selected from:

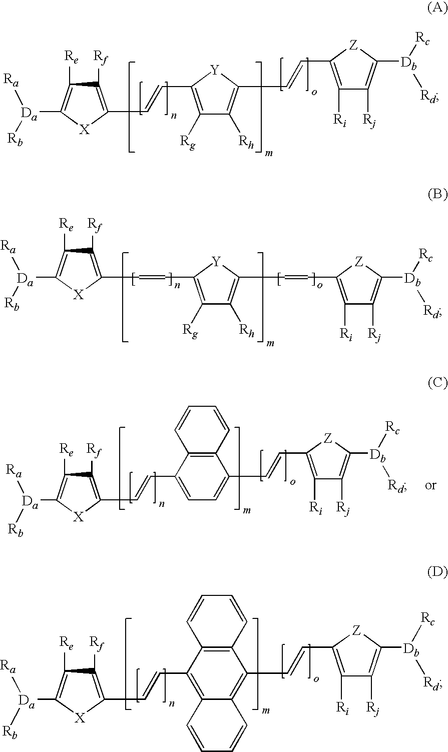

wherein:
Dₐ is selected from N, O, S, or P;
D_{b} is selected from N, O, S, or P;
m, n, and o independently are integers from 0 to 10, inclusive;
X, Y, and Z independently are selected from CRₖ═CRₗ, O, S, or N—Rₘ;
Rₐ, R_{b}, R_{c}, and R_{d} independently are selected from a hydrogen atom, a linear or branched alkyl group with up to 25 carbons, —(CH₂CH₂O)_{α}—(CH₂)_{β}ORₐ₁, —(CH₂CH₂O)_{α}—(CH₂)_{β}NRₐ₂Rₐ₃, —(CH₂CH₂O)_{α}—(CH₂)_{β}CONRₐ₂Rₐ₃, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, a group of aromatic rings having up to 20 carbons in the aromatic ring framework, fused aromatic rings, vinyl, allyl, 4-styryl, acroyl, methacroyl, acrylonitrile, isocyanate, isothiocyanate, epoxides, strained ring olefins, (—CH₂)_{γ}SiCl₃, (—CH₂)_{γ}Si(OCH₂CH₃)₃, or (—CH₂)_{γ}Si(OCH₃)₃; wherein one of Rₐ and R_{b} is not present when Dₐ is O or S and wherein one of R_{c} and R_{d} is not present when D_{b} is O or S;
Rₑ, R_{f}, R_{g}, Rₕ, Rᵢ, Rⱼ, Rₖ, Rₗ, and Rₘ independently are selected from a hydrogen atom, a linear or branched alkyl group with up to 25 carbons, —(CH₂CH₂O)_{α}—(CH₂)_{β}OR_{b1}, —(CH₂CH₂O)_{α}—(CH₂)_{β}NR_{b2}R_{b3}, —(CH₂CH₂O)_{α}—(CH₂)_{β}CONR_{b2}R_{b3}, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, a group of aromatic rings having up to 20 carbons in the aromatic framework, fused aromatic rings, CHO, CN, NO₂, Br, Cl, I, phenyl, an acceptor group containing more than two carbon atoms, a functional group obtained by reaction with an amino acid,
NRₑ₁Rₑ₂, or ORₑ₃;
Rₐ₁, Rₐ₂, and Rₐ₃ independently are selected from a hydrogen atom, a linear or branched alkyl group with up to 25 carbons, or a functional group obtained by reaction with: an amino acid, a polypeptide, adenine, guanine, tyrosine, cytosine, uracil, biotin, ferrocene, ruthenocene, cyanuric chloride, or methacryloyl chloride;
R_{b1}, R_{b2}, and R_{b3} independently are selected from a functional group obtained by reaction with: an amino acid, a polypeptide, adenine, guanine, tyrosine, cytosine, uracil, biotin, ferrocene, ruthenocene, cyanuric chloride, or methacryloyl chloride;
Rₑ₁, Rₑ₂, and Rₑ₃, are independently selected from a hydrogen atom, a linear or branched alkyl group with up to 25 carbons, —(CH₂CH₂O)_{α}—(CH₂)_{β}OR_{g1}, —(CH₂CH₂O)_{α}—(CH₂)_{β}NR_{g2}R_{g3}, —(CH₂CH₂O)_{α}—(CH₂)_{β}CONR_{g2}R_{g3}, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}, —(CH₂)_{β}-Phenyl, aryl groups, fused aromatic rings, vinyl, allyl, 4-styryl, acroyl, methacroyl, acrylonitrile, isocyanate, isothiocyanate, epoxides, strained ring olefins, (—CH₂)_{γ}SiCl₃, (—CH₂)_{γ}Si(OCH₂CH₃)₃, or (—CH₂)_{γ}Si(OCH₃)₃;
R_{g1}, R_{g2}, and R_{g3} independently are selected from a hydrogen atom, a linear or branched alkyl group with up to 25 carbons, or a functional group obtained by reaction with: an amino acid, a polypeptide, adenine, guanine, tyrosine, cytosine, uracil, biotin, ferrocene, ruthenocene, cyanuric chloride, or methacryloyl chloride;
α is an integer from 0 to 10, inclusive;
β and γ independently are integers in a range from 1 to 25, inclusive; and
   (b) at least one second compound having at least one photocleavable group bonded to at least one protected group,
wherein the at least one photocleavable group has the formula:

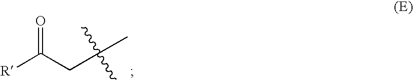

wherein R′ is a substituted or unsubstituted aryl or heteroaryl moiety, wherein any substituents on R′ are selected from alkyl, alkenyl, alkoxy, or hydroxy groups; and wherein the at least one protected group is selected from selected from:

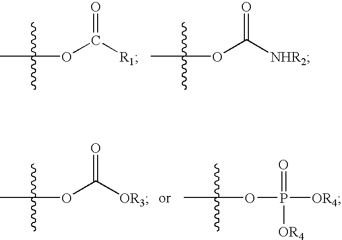

wherein:
   R₁, R₂, R₃, and R₄ independently are selected from a substituted or unsubstituted alkyl, alkenyl, alkynyl, aryl, aralkyl, aralkenyl, aralkynyl, fused aryl, carbocyclic, carboxylalkyl-substituted alkyl, heterocycloalkyl, heterocycloalkyl-substituted alkyl, heteroaryl, heteroarylalkyl, heteroarylalkenyl, or heteroaralkynyl groups, wherein any substituents on R₁, R₂, R₃, and R₄ independently are selected from alkyl, alkenyl, alkynyl, alkoxy, acyl (alkanoyl), acyloxy, cyano, alkylcarboxy, chloro, bromo, aryl, cycloalkyl, aralkyl, aralkenyl, aralkynyl, hydroxy, nitro, amino, carboxy, amino acid, peptide, adenine, guanine, tyrosine, cytosine, uracil, biotin, ferrocene, ruthenocene, cyanuric chloride, methacryloyl chloride, glucose, mannose, galactose, gulose, allose, altrose, idose, talose, fructose, arabinose, xylose, sucrose, cellobiose, maltose, lactose, trehalose, gentiobiose, melibiose, raffinose, gentianose, adenosine, deoxyadenosine, guanosine, deoxyguanosine, cytidine, deoxycytidine, uridine, or deoxythymidine.

In another aspect, R₁, R₂, R₃, and R₄ independently are selected from alkoxy, acyl (alkanoyl), acyloxy, cyano, alkylcarboxy, chloro, bromo, cycloalkyl, hydroxy, nitro, amino, carboxy, amino acid, peptide, adenine, guanine, tyrosine, cytosine, uracil, biotin, ferrocene, ruthenocene, cyanuric chloride, methacryloyl chloride, glucose, mannose, galactose, gulose, allose, altrose, idose, talose, fructose, arabinose, xylose, sucrose, cellobiose, maltose, lactose, trehalose, gentiobiose, melibiose, raffinose, gentianose, adenosine, deoxyadenosine, guanosine, deoxyguanosine, cytidine, deoxycytidine, uridine, or deoxythymidine.

Useful alkyls in the at least one second compound include C₁-C₁₆ linear or branched alkyl groups. Alternatively, any alkyl group in the second compound is a C₁-C₈ linear or branched alkyl. Suitable alkyls include, but are not limited to, methyl, ethyl, propyl, isopropyl, butyl, sec-butyl, tert-butyl, 3-pentyl, hexyl, octyl, and the like. Further, the alkyl groups optionally can be substituted.

Alkenes suitable for use in the at least one second compound generally are C₂-C₁₀ linear or branched alkenyl groups. In another aspect, the alkenyl can be a linear C₂-C₆ alkenyl group. Ethenyl, propenyl, isopropenyl, butenyl, sec-butenyl, 3-pentenyl, hexenyl, octenyl, and the like, are suitable alkenes of the present invention. Optionally, the alkenyl group can be substituted.

Likewise, alkynes suitable for use in the at least one second compound are C₂-C₁₀ linear or branched alkynyl groups. Alternatively, the alkynyl can be a linear C₂-C₆ alkynyl group. Suitable alkynyls include, but are not limited to, ethynyl, propynyl, butynyl, pentynyl, hexynyl, octynyl, and the like, all of which can be optionally substituted.

Alkoxy groups can include, but are not limited to, an oxygen group substituted with a C₁-C₁₆ linear or branched alkyl groups, as described above. The alkyl group can be optionally substituted.

In accordance with the present invention, amino groups can include, but not limited to, —NH₂, —NHR₅, and —NR₆R₇, wherein R₅, R₆, and R₇ are C₁-C₁₀ alkyl or cycloalkyl groups, or R₆ and R₇ are combined with the N atom to form a ring structure, such as a piperidine, or R₆ and R₇ are combined with the N atom and another group to form a ring, such as a piperazine. The alkyl and cycloalkyl groups optionally can be substituted.

Suitable aryls include C₆-C₁₄ aryl groups, or in the alternative, C₆-C₁₀ aryl groups. Typical aryl groups that can be used in accordance with the present invention include, but are not limited to, phenyl, naphthyl, phenanthrenyl, anthracenyl, indenyl, azulenyl, biphenyl, biphenylenyl, fluorenyl, and the like.

Cycloalkyls that are useful in the present invention generally have from 3 to 8 carbon atoms, respectively. Non-limiting examples of suitable cycloalkyls include cyclopropyl, cyclobutyl, cyclopentyl, cyclohexyl, and cycloheptyl. In addition to these saturated carbocyclic groups, unsaturated carbocyclics including, but not limited to, cyclopentenyl, cycloheptenyl, cyclooctenyl, and the like, can be employed.

Arylalkyls generally can include the aforementioned C₁-C₁₆ linear or branched alkyl groups substituted by any of the aforementioned C₆-C₁₄ aryl groups. Non-limiting examples include benzyl, phenylethyl, naphthylmethyl, and the like.

Acyloxy groups that can be used in the present invention include, but not limited to a C₁-C₆ acyl (alkanoyl) attached to an oxy (—O—) group, such as, for example, formyloxy, acetoxy, propionoyloxy, butanoyloxy, pentanoyloxy, hexanoyloxy, and the like.

Useful saturated or partially saturated heterocyclic groups include, but are not limited to, tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl pyrazolinyl, tetronoyl, tetramoyl, and the like.

Heteroaryl groups can be employed in the present invention. These can include, but are not limited to, thienyl, benzo[b]thienyl, naphtho[2,3-b]thienyl, thianthrenyl, furyl, pyranyl, isobenzofuranyl, chromenyl, xanthenyl, phenoxanthiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, carbolinyl, phenanthridinyl, acrindinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, 1,2-benzoisoxazol-3-yl, benzimidazolyl, 2-oxindolyl, 2-oxobenzimidazolyl, and the like.

Sugars which can be protected functional groups of the present invention include, but are not limited to, glucose, mannose, galactose, gulose, allose, altrose, idose, talose, fructose, arabinose, xylose, sucrose, cellobiose, maltose, lactose, trehalose, gentiobiose, melibiose, raffinose, gentianose, and the like.

In one aspect, a primary hydroxy group is selectively protected with about one equivalent of an aryl or heteroaryl acyl alcohol. Alternatively, substantially all of the hydroxy groups can be protected by treatment with a large excess of aryl or heteroaryl acyl alcohol and other similar condensing reagents, as is known to those of skill in the art.

Examples of RNA and DNA bases that may be protected at the 5′-position according to the present invention include, but are not limited to, adenosine, deoxyadenosine, guanosine, deoxyguanosine, cytidine, deoxycytidine, uridine, deoxythymidine, and the like. The phosphate groups of the corresponding mono-, di- and triphosphates (e.g., at the 3′- or 5′-position) and cyclic phosphates (e.g., cyclic AMP) can also be protected according to the present invention using methods analogous to those employed in the solid phase synthesis of oligonucleotides, e.g., with the corresponding cyanoethyl phosphoramidates of the protected bases (where necessary). This technology is disclosed in Eckstein, F., Oligonucleotides and Analogs—A Practical Approach, IRL Press, New York, N.Y. (1991).

Often, compositions as described above, are formed in mixtures, solutions, suspensions, emulsions, and other types of blends, as would be recognized by those of skill in the art. In a composition comprising at least one chromophore compound and at least one second compound having at least one photocleavable group bonded to at least one protected group, the electrostatic interaction (also referred to an the energy of attraction) between the at least one chromophore compound and the at least one second compound generally can be greater than 3 kcal/mol. In another aspect, the electrostatic interaction is greater than 5 kcal/mol, or greater than 10 kcal/mol. This electrostatic interaction, or energy of attraction, applies when the at least one chromophore compound and the at least one second compound, independently, are present in a solution at a concentration between about 0.001M and about 2M. Yet, in another aspect, the concentration of the at least one chromophore compound and the at least one second compound, independently, are present in a solution at a concentration between about 0.01M and about 1M.

Compositions as described above can be used in a method for deprotecting a protected functional group. Such a method comprises:

(a) providing a composition, the composition comprising at least one chromophore compound and at least one second compound having at least one photocleavable group bonded to at least one protected group,

(b) exposing the composition to radiation;

(c) converting the chromophore compound to a multi-photon electronically excited state upon simultaneous absorption of at least two photons of the radiation by the chromophore compound, wherein the sum of the energies of all of the absorbed photons is greater than or equal to the transition energy from a ground state of the chromophore compound to the multi-photon excited state and wherein the energy of each absorbed photon is less than the transition energy between the ground state and the lowest single-photon excited state of the chromophore compound and is less than the transition energy between the multi-photon excited state and the ground state;

thereby inducing an electron transfer from the chromophore compound to the photocleavable group in the second compound to cleave and deprotect the protected functional group in the second compound.

As disclosed in U.S. Pat. No. 6,392,089, photocleavable groups are well known to those of skill in the art, as are their attachment to protected functional groups.

Protected functional groups can include, but are not limited to, carbonyl groups, amines, alcohols and phenols, phosphates, and the like. Protected functional groups are well known to those of skill in the art, and are disclosed in U.S. Pat. No. 6,392,089. In the present invention, the protected functional groups (shown above) are bonded to the photocleavable group through the oxygen atom of the protected functional group.

In accordance with the present invention, second compounds containing at least photocleavable group and at least one protected functional group can be produced by any means known to those of skill in the art. In one aspect, Reaction Scheme A illustrates a synthesis scheme for compounds containing carboxylic acids functional groups protected by photocleavable groups:

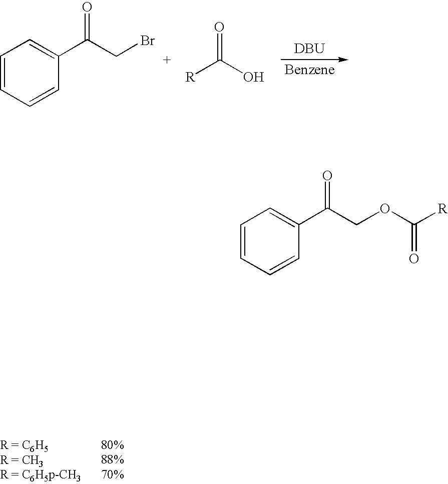

When R is CH₃, the resulting compound is acetic acid 2-oxo-2-phenyl-ethyl ester, also referred to as 2-oxo-2-phenylethyl acetate, and is shown below as Compound (1):

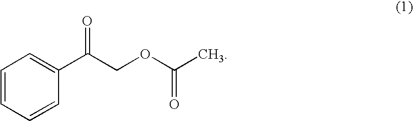

A non-limiting example of the synthesis of Compound (1) is provided in Example 1.

Other illustrative, and non-limiting, examples of reaction schemes that can be used in the present invention to produce specific compounds containing at least one photocleavable group and at least one protected functional group are provided below:

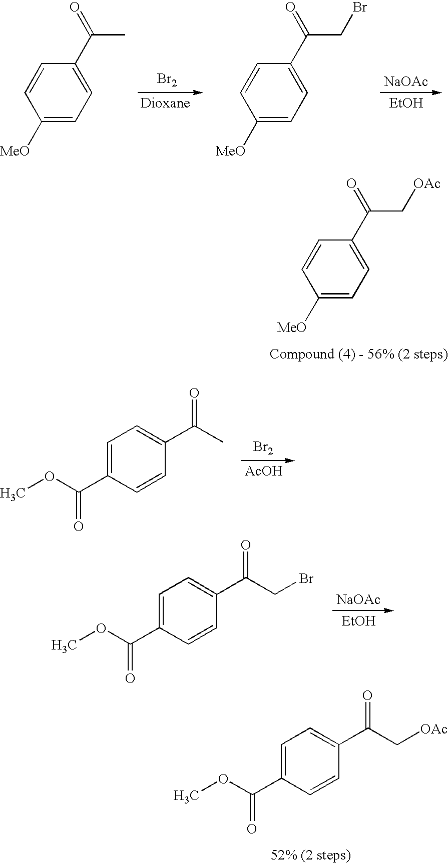

Suitable chromophore compounds of the present invention can include, but are not limited to,

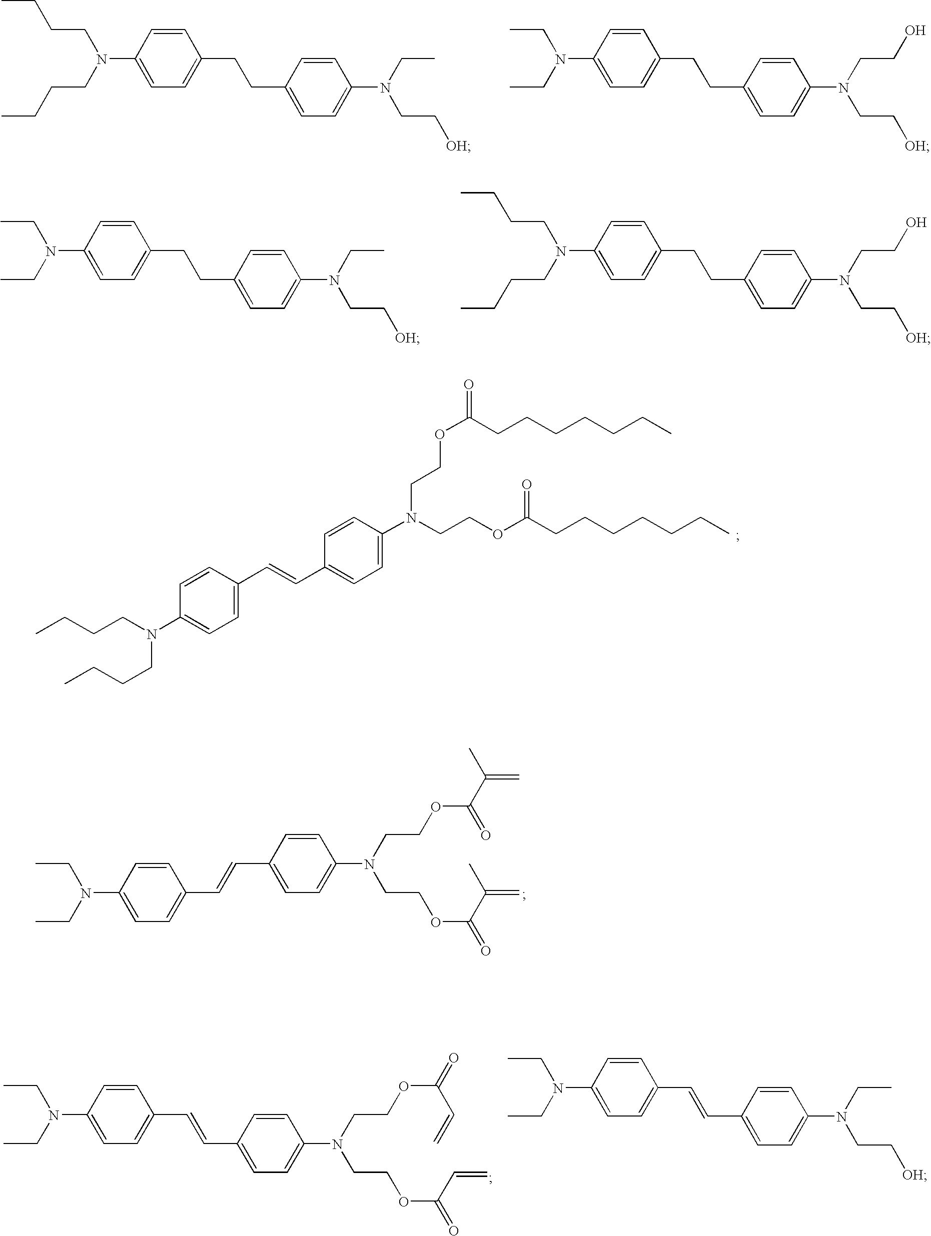

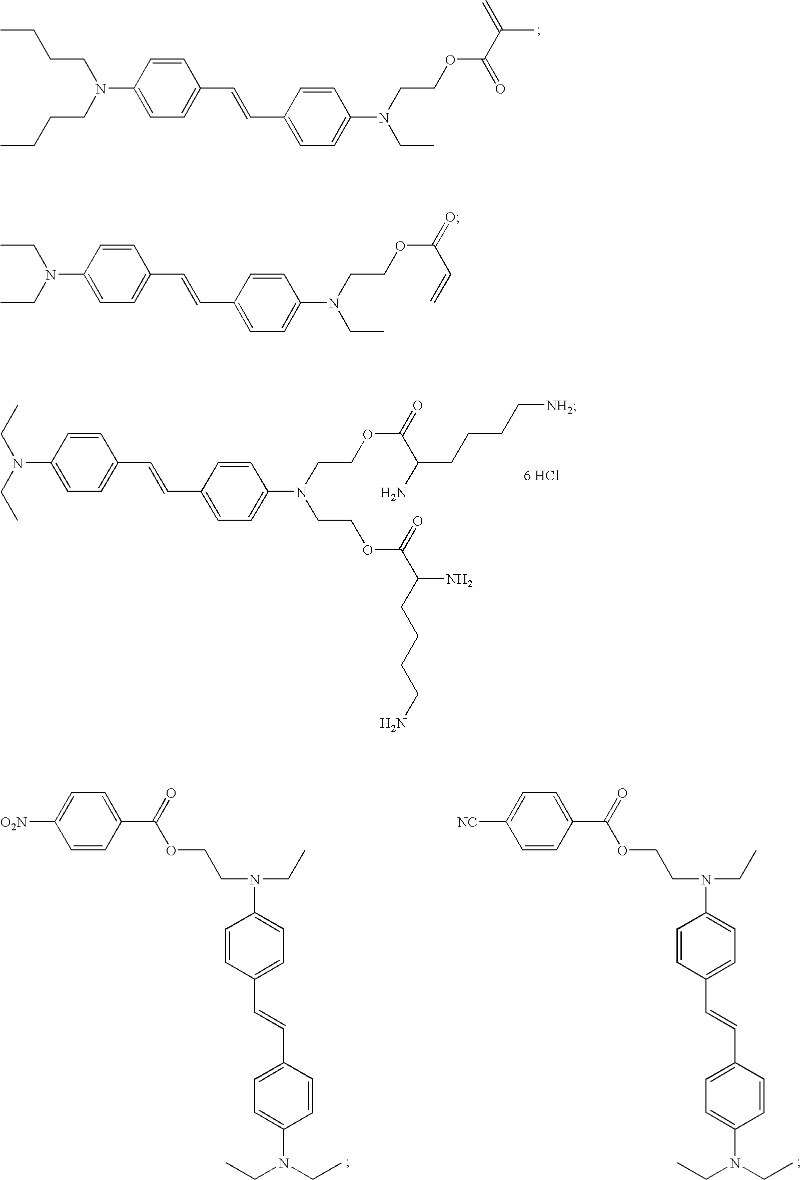

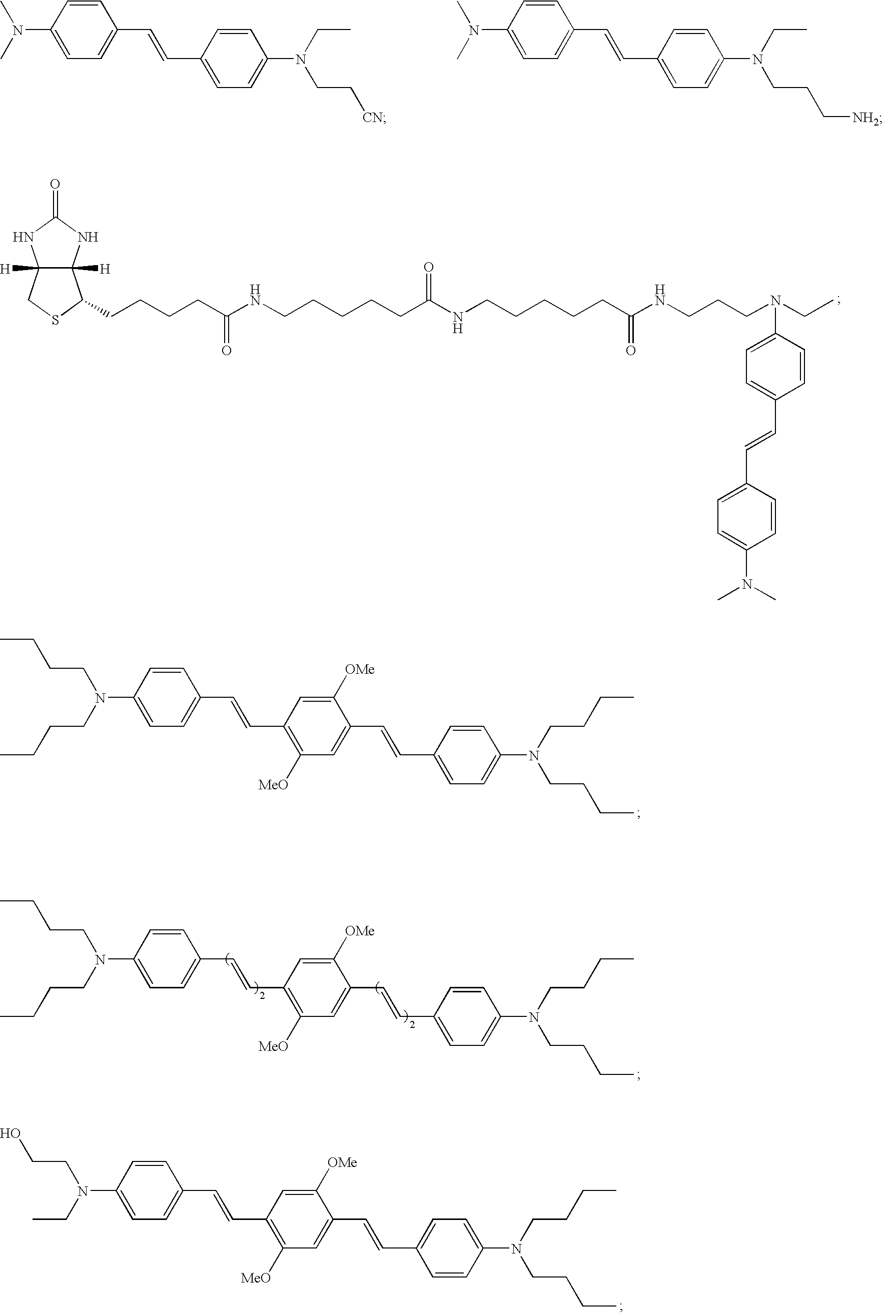

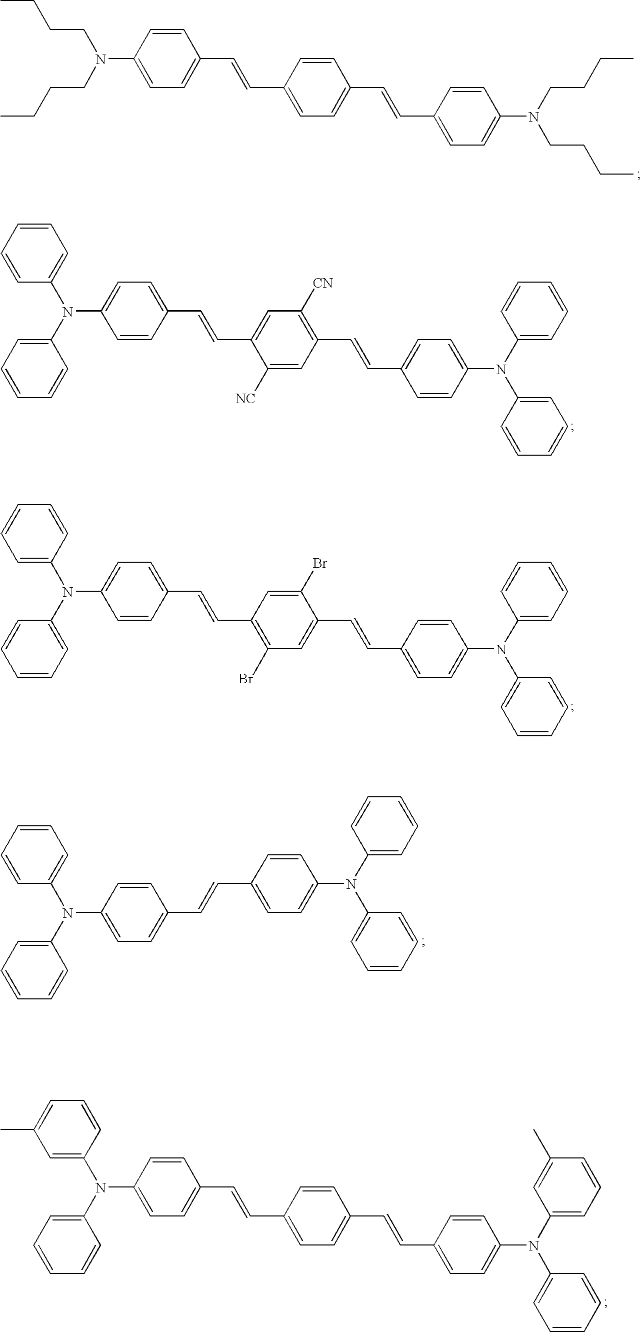

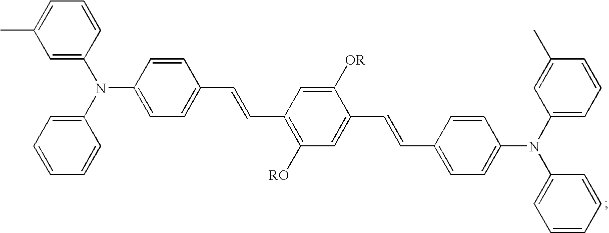

and the like, or combinations thereof, wherein R═(CH₂)₁₁CH₃.

A composition in accordance with the present invention can include at least one chromophore compound selected from the species presented above and at least one second compound. The at least one second compound has at least one photocleavable group bonded to at least one protected group. The at least one photocleavable group has formula (E), wherein R′ is defined above. The at least one protected group is selected from selected from:

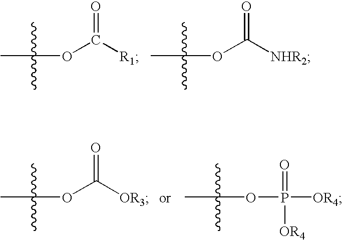

wherein R₁, R₂, R₃, and R₄ are as defined above.

In another aspect, this invention also provides reagents of the molecular formula Ar—C(R₁)(R₂)—O—C(O)—X₂, where Ar, R₁, and R₂ have the meanings ascribed above, for incorporating the protecting group into the molecule desired to be protected. X₂ can be any suitable leaving group such as halo, oxycarbonyl, imidazolyl, pentafluorophenoxy and the like, which is capable of reacting with a nucleophilic group such as hydroxy, amino, alkylamino, thio and the like on the molecule being protected. Thus, the reagents comprising the protecting groups Ar—C(R₁)(R₂)—O—C(O)— disclosed herein can be used in numerous applications where protection of a reactive nucleophilic group is required. Such applications include, but are not limited to polypeptide synthesis, both solid phase and solution phase, oligo- and polysaccharide synthesis, polynucleotide synthesis, protection of nucleophilic groups in organic syntheses of potential drugs, etc.

The invention also provides compositions of the molecular formula Ar—C(R₁)(R₂)—O—C(O)-M, where Ar, R₁ and R₂ have the meaning outlined above and M is any other chemical fragment. Preferably, M will be a monomeric building block that can be used to make a macromolecule. Such building blocks include amino acids, peptides, polypeptides, nucleic acids, nucleotides, nucleosides, monosaccharides, and the like. Preferred nucleosides are ribonucleosides and deoxyribonucleosides such as adenosine, deoxyadenosine, cytidine, deoxycytidine, thymidine, uracil, guanosine and deoxyguanosine as welt as oligonucleotides incorporating such nucleosides. Preferably, the building block is linked to the photolabile protecting group via a hydroxy or amine group. When nucleotide and oligonucleotide compositions are used, with the protecting groups of this invention, the protecting groups are preferably incorporated into the 3′-OH or the 5′-OH of the nucleoside. Other preferred compounds are protected peptides, proteins, oligonucleotides and oligodeoxyribonucleotides. Small organic molecules, proteins, hormones, antibodies and other such species having nucleophilic reactive groups can be protected using the protecting groups disclosed herein.

The use of nucleoside and nucleotide analogs is also contemplated by this invention to provide oligonucleotide or oligonucleoside analogs bearing the protecting groups disclosed herein. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into an oligonucleotide or oligonucleoside sequence, they allow hybridization with a naturally occurring oligonucleotide sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Analogs also include protected and/or modified monomers as are conventionally used in oligonucleotide synthesis. As one of skill in the art is well aware oligonucleotide synthesis uses a variety of base-protected deoxynucleoside derivatives in which one or more of the nitrogens of the purine and pyrimidine moiety are protected by groups such as dimethoxytrityl, benzyl, tert-butyl, iso-butyl and the like. Specific monomeric building blocks which are encompassed by this invention include base protected deoxynucleoside H-phosphonates and deoxynucleoside phosphoramidites.

For instance, structural groups are optionally added to the ribose or base of a nucleoside for incorporation into an oligonucleotide, such as a methyl, propyl or allyl group at the 2′-O position on the ribose, or a fluoro group which substitutes for the 2′-O group, or a bromo group on the ribonucleoside base. 2′-O-methyloligoribonucleotides (2′-O-MeORNs) have a higher affinity for complementary nucleic acids (especially RNA) than their unmodified counterparts. 2′-O-MeORNA phosphoramidite monomers are available commercially, e.g., from Chem Genes Corp. or Glen Research, Inc. Alternatively, deazapurines and deazapyrimidines in which one or more N atoms of the purine or pyrimidine heterocyclic ring are replaced by C atoms can also be used.

The phosphodiester linkage, or “sugar-phosphate backbone” of the oligonucleotide analogue can also be substituted or modified, for instance with methyl phosphonates or O-methyl phosphates. Another example of an oligonucleotide analogue for purposes of this disclosure includes “peptide nucleic acids” in which a polyamide backbone is attached to oligonucleotide bases, or modified oligonucleotide bases. Peptide nucleic acids which comprise a polyamide backbone and the bases found in naturally occurring nucleosides are commercially available from, e.g., Biosearch, Inc. (Bedford, Mass.).

Nucleotides with modified bases can also be used in this invention. Some examples of base modifications include 2-aminoadenine, 5-methylcytosine, 5-(propyn-1-yl)cytosine, 5-(propyn-1-yl)uracil, 5-bromouracil, and 5-bromocytosine which can be incorporated into oligonucleotides in order to increase binding affinity for complementary nucleic acids. Groups can also be linked to various positions on the nucleoside sugar ring or on the purine or pyrimidine rings which may stabilize the duplex by electrostatic interactions with the negatively charged phosphate backbone, or through hydrogen bonding interactions in the major and minor groves. For example, adenosine and guanosine nucleotides can be substituted at the N² position with an imidazolyl propyl group, increasing duplex stability. Universal base analogues such as 3-nitropyrrole and 5-nitroindole can also be included. A variety of modified oligonucleotides and oligonucleotide analogs suitable for use in this invention are described in, e.g., “Antisense Research and Applications”, S. T. Crooke and B. LeBleu (eds.) (CRC Press, 1993) and “Carbohydrate Modifications in Antisense Research” in ACS Symp. Ser. #580, Y. S. Sanghvi and P. D. Cook (eds.) ACS, Washington, D.C. 1994).

Compounds of this invention can be prepared by carbonylating an aromatic carbinol of the general formula Ar—C(R₁)(R₂)—OH with a carbonylation reagent such as for example, phosgene (COCl₂), carbonyldiimidazole or pentafluorophenoxy chloroformate and the like to provide Ar—C(R₁)(R₂)—O—C(O)—X₃ where X₃ is a leaving group derived from the carbonylating reagent (Cl, if phosgene was used, pentafluorophenoxy, if pentafluorophenoxy chloroformate was used, etc.). This intermediate, Ar—C(R₁)(R₂)—O—C(O)—X₃ is then reacted with a molecule M carrying a nucleophilic group whose protection is desired to yield a protected building block Ar—C(R₁)(R₂)—O—C(O)-M. Representative aromatic carbinols are pyrenemethanol, naphthalenemethanol, anthracenemethanol, perylenemethanol and the like. Such aromatic carbinols are available from commercial suppliers such as Aldrich Chemical Co., Milwaukee, Wis. Alternatively, they may also be obtained from precursor aromatic hydrocarbons by acylation under Friedel-Crafts conditions with acid chlorides and anhydrides and subsequent reduction of the carbonyl group thus added to a carbinol.

Alternatively, one may first carbonylate the group on the molecule being protected with a carbonylation reagent, such as one described above, and subsequently displace the leaving group X₃ thus inserted with the hydroxyl group of the aromatic carbinol. In either procedure, one frequently uses a base such as triethylamine or diisopropylethylamine and the like to facilitate the displacement of the leaving group.

One of skill in the art will recognize that the protecting groups disclosed herein can also be attached to species not traditionally considered as “molecules”. Therefore, compositions such as solid surfaces (e.g., paper, nitrocellulose, glass, polystyrene, silicon, modified silicon, GaAs, silica and the like), gels (e.g., agarose, sepharose, polyacrylamide and the like) to which the protecting groups disclosed herein are attached are also contemplated by this invention.

A molecule with a reactive site may be attached to a support, following the steps of:

(a) providing a support with a reactive site;
(b) binding a molecule to the reactive site, the first molecule comprising a masked reactive site attached to a photolabile protecting group of the formula Ar—C(R₁)(R₂)—O—C(O)—, wherein:

Ar is an optionally substituted fused polycyclic aryl or heteroaromatic group or a vinylogously substituted derivative of the foregoing;

R₁ and R₂ are independently H, optionally substituted alkyl, alkenyl or alkynyl, or optionally substituted aryl or heteroaromatic group or a vinylogously substituted derivative of the foregoing;

to produce a derivatized support having immobilized thereon the molecule attached to the photolabile protecting group; and

(c) removing the photolabile protecting group to provide a derivatized support comprising the molecule with an unmasked reactive site immobilized thereon.

As one of skill will recognize, the process can be repeated to generate a compound comprising a chain of component molecules attached to the solid support. In a “mix and match” approach, the photolabile protecting groups may be varied at different steps in the process depending on the ease of synthesis of the protected precursor molecule. Alternatively, photolabile protecting groups can be used in some steps of the synthesis and chemically labile (e.g. acid or base sensitive groups) can be used in other steps, depending for example on the availability of the component monomers, the sensitivity of the substrate and the like.

Specific examples of groups that can be used in this manner include those shown in U.S. Pat. No. 6,310,083, but are not limited to:

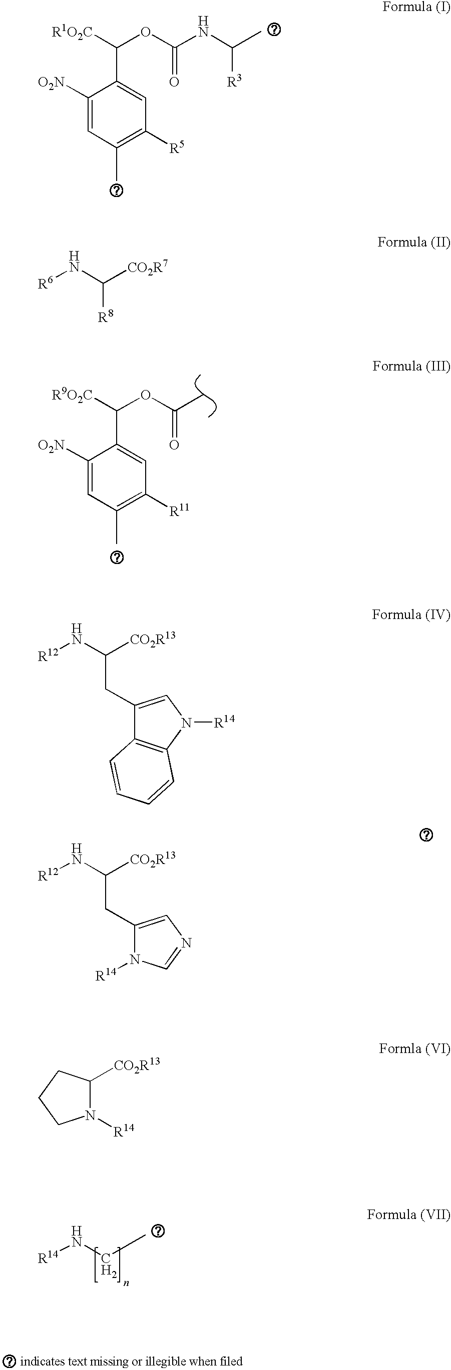

In Formulae (I)-(VII), the substituent groups are further defined as follows:

R¹ and R² are preferably each selected from the group consisting of H, Na, K, methyl, ethyl, and t-butyl;

R³ is preferably selected from the group consisting of H, CH₃, —CH(CH₃)₂, —CH₂—CH(CH₃)₂, —CH₂—CH(CH₃)(CH₂CH₃), —CH₂CH₂SCH₃, —CH₂—C₆H₅, —CH₂CO₂H, —CH₂CONH₂, —CH₂CH₂CO₂H, and —CH₂CH₂CONH₂;

R⁴ and R⁵ are preferably each H or —OCH₃, or combined together to form —OCH₂O—;

R⁶ is preferably selected from the group consisting of 9-fluorenylmethyloxycarbonyl (Fmoc), t-butyloxycarbonyl (Boc), benzyloxycarbonyl (Cbz), and t-butyl-o-nitromandelyloxycarbonyl (t-butyl Nmoc);

R⁷ is preferably selected from the group consisting of H, Na, K, methyl, ethyl, and t-butyl;

R⁸ is preferably selected from the group consisting of (t-butyl-o-nitromandelyloxycarbonyl)-OCH₂—, (t-butyl-o-nitromandelyloxycarbonyl)-OCH(CH₃)—, (t-butyl-o-nitromandelyloxycarbonyl)-SCH₂—, (t-butyl-o-nitromandelyloxycarbonyl)-NH(CH₂)₄—, (t-butyl-o-nitromandelyloxycarbonyl)-NH—C(═NH)—NH(CH₂)₃—, and (t-butyl-o-nitromandelyloxycarbonyl)-O—C₆H₄—CH₂—;

R⁹ is preferably selected from the group consisting of H, CH₃, and t-butyl;

R¹⁰ and R¹¹ are preferably each H or —OCH₃, or combined together to form —OCH₂O—;

R¹² is preferably selected from the group consisting of 9-fluorenylmethyloxycarbonyl (Fmoc), t-butyloxycarbonyl (Boc), benzyloxycarbonyl (Cbz), and t-butyl-o-nitromandelyloxycarbonyl (t-butyl Nmoc);

R¹³ is preferably selected from the group consisting of H, CH₃, and t-butyl; and

R¹⁴ is preferably t-butyl-o-nitromandelyloxycarbonyl (t-butyl Nmoc);

Acetoxymethyl (—CH₂O₂CCH₃) (AM) esters can be directly loaded into living cells. This is because these esters mask the negative charge on the carboxyl group, and the resulting compounds are neutral and hydrophobic, such that they easily diffuse across biological membranes. Once inside the cells, however, the esters are readily hydrolyzed by non-specific esterases to yield the caged amino acid compound, which are negatively charged, and unable to cross biological membranes, and thus become trapped and accumulate inside the cells.

Specific examples of the compounds of the present invention include N-(t-butyl Nmoc)-glycine, N-(t-butyl Nmoc)-L-alanine, N-(t-butyl Nmoc)-D-alanine, N-(t-butyl Nmoc)-L-valine, N-(t-butyl Nmoc)-D-valine, N-(t-butyl Nmoc)-L-leucine, N-(t-butyl Nmoc)-D-leucine, N-(t-butyl Nmoc)-L-isoleucine, N-(t-butyl Nmoc)-D-isoleucine, N-(t-butyl Nmoc)-L-methionine, N-(t-butyl Nmoc)-D-methionine, N-(t-butyl Nmoc)-L-phenylalanine, N-(t-butyl Nmoc)-D-phenylalanine, α-N-(t-butyl Nmoc)-L-aspartic acid, α-N-(t-butyl Nmoc)-D-aspartic acid, α-N-(t-butyl Nmoc)-L-asparagine, α-N-(t-butyl Nmoc)-D-asparagine, α-N-(t-butyl Nmoc)-L-glutamic acid, α-N-(t-butyl Nmoc)-D-glutamic acid, α-N-(t-butyl Nmoc)-L-glutamine, α-N-(t-butyl Nmoc)-D-glutamine, N-(t-butyl Nmoc)-L-proline, N-(t-butyl Nmoc)-D-proline, α-N-Fmoc-.epsilon.-N-(t-butyl Nmoc)-L-lysine, α-N-Fmoc-.epsilon.-N-(t-butyl Nmoc)-D-lysine, α-N-Fmoc-N^{G}-(t-butyl Nmoc)-L-arginine, α-N-Fmoc-N^{G}-(t-butyl Nmoc)-D-arginine, α-N-Fmoc-S-(t-butyl Nmoc)-L-cysteine, α-N-Fmoc-S-(t-butyl Nmoc)-D-cysteine, α-N-Fmoc-β-O-(t-butyl Nmoc)-L-serine, α-N-Fmoc-β-O-(t-butyl Nmoc)-D-serine, α-N-Fmoc-β-O-(t-butyl Nmoc)-L-threonine, α-N-Fmoc-β-O-(t-butyl Nmoc)-D-threonine, α-N-Fmoc-4-O-(t-butyl Nmoc)-L-tyrosine, α-N-Fmoc-4-O-(t-butyl Nmoc)-D-tyrosine, α-N-Fmoc-N^{ln}-(t-butyl Nmoc)-L-tryptophan, α-N-Fmoc-N^{ln}-(t-butyl Nmoc)-D-tryptophan, α-N-Fmoc-N^{lm}-(t-butyl Nmoc)-L-histidine, α-N-Fmoc-N^{lm}-(t-butyl Nmoc)-D-histidine, N-Nmoc-glycine, N-Nmoc-L-alanine, N-Nmoc-D-alanine, N-Nmoc-L-valine, N-Nmoc-D-valine, N-Nmoc-L-leucine, N-Nmoc-D-leucine, N-Nmoc-L-isoleucine, N-Nmoc-D-isoleucine, N-Nmoc-L-methionine, N-Nmoc-D-methionine, N-Nmoc-L-phenylalanine, N-Nmoc-D-phenylalanine, α-N-Nmoc-L-aspartic acid, α-N-Nmoc-D-aspartic acid, α-N-Nmoc-L-asparagine, α-N-Nmoc-D-asparagine, α-N-Nmoc-L-glutamic acid, α-N-Nmoc-D-glutamic acid, α-N-Nmoc-L-glutamine, α-N-Nmoc-D-glutamine, N-Nmoc-L-proline, N-Nmoc-D-proline, .epsilon.-N-Nmoc-L-lysine, .epsilon.-N-Nmoc-D-lysine, N^{G}-Nmoc-L-arginine, N^{G}-Nmoc-D-arginine, S-Nmoc-L-cysteine, S-Nmoc-D-cysteine, β-O-Nmoc-L-serine, β-O-Nmoc-D-serine, β-O-Nmoc-L-threonine, β-O-Nmoc-D-threonine, 4-O-Nmoc-L-tyrosine, 4-O-Nmoc-D-tyrosine, N^{ln}-Nmoc-L-tryptophan, N^{ln}-Nmoc-D-tryptophan, N^{lm}-Nmoc-L-histidine, N^{lm}-Nmoc-D-histidine, and N-Nmoc-4-aminobutyric acid.

### Intramolecular Photodeprotection

Intramolecular photodeprotection involves substantially the same procedure discussed above relative to intermolecular photodeprotection, with the exception that a single compound is employed. Such a compound comprises at least one chromophore moiety bonded to at least one photocleavable group, the at least one photocleavable group bonded to at least one protected group, wherein:

(a) the at least one chromophore moiety is selected from:

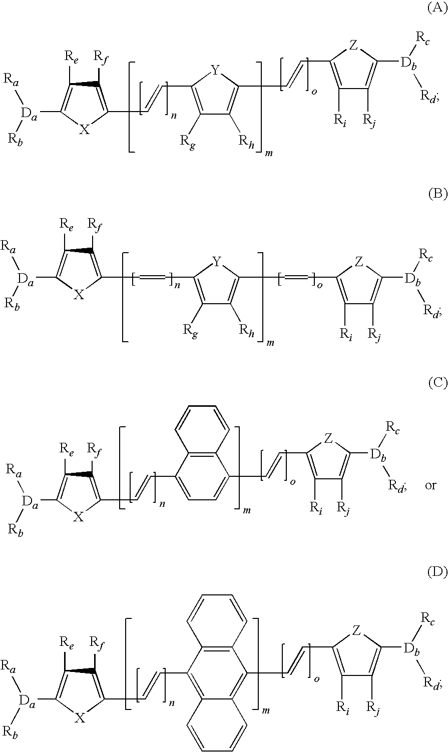

wherein:
Dₐ is selected from N, O, S, or P;
D_{b} is selected from N, O, S, or P;
m, n, and o independently are integers from 0 to 10, inclusive;
X, Y, and Z independently are selected from CRₖ═CRₗ, O, S, or N—Rₘ;
Rₐ, R_{b}, R_{c}, and R_{d} independently are selected from a hydrogen atom, a linear or branched alkyl group with up to 25 carbons, —(CH₂CH₂O)_{α}—(CH₂)_{β}ORₐ₁, —(CH₂CH₂O)_{α}—(CH₂)_{β}NRₐ₂Rₐ₃, —(CH₂CH₂O)_{α}—(CH₂)_{β}CONRₐ₂Rₐ₃, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, a group of aromatic rings having up to 20 carbons in the aromatic ring framework, fused aromatic rings, vinyl, allyl, 4-styryl, acroyl, methacroyl, acrylonitrile, isocyanate, isothiocyanate, epoxides, strained ring olefins, (—CH₂)_{γ}SiCl₃, (—CH₂)_{γ}Si(OCH₂CH₃)₃, or (—CH₂)_{γ}Si(OCH₃)₃; wherein one of Rₐ and R_{b} is not present when Dₐ is O or S and wherein one of R_{c} and R_{d} is not present when D_{b} is O or S;
Rₑ, R_{f}, R_{g}, Rₕ, Rᵢ, Rⱼ, Rₖ, Rₗ, and Rₘ independently are selected from a hydrogen atom, a linear or branched alkyl group with up to 25 carbons, —(CH₂CH₂O)_{α}—(CH₂)_{β}OR_{b1}, —(CH₂CH₂O)_{α}—(CH₂)_{β}NR_{b2}R_{b3}, —(CH₂CH₂O)_{α}—(CH₂)_{β}CONR_{b2}R_{b3}, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, a group of aromatic rings having up to 20 carbons in the aromatic framework, fused aromatic rings, CN, NO₂, Br, Cl, I, phenyl, an acceptor group containing more than two carbon atoms, a functional group obtained by reaction with an amino acid, NRₑ₁Rₑ₂, or ORₑ₃;
Rₐ₁, Rₐ₂, and Rₐ₃ independently are selected from a hydrogen atom, a linear or branched alkyl group with up to 25 carbons, or a functional group obtained by reaction with: an amino acid, a polypeptide, adenine, guanine, tyrosine, cytosine, uracil, biotin, ferrocene, ruthenocene, cyanuric chloride, or methacryloyl chloride;
R_{b1}, R_{b2}, and R_{b3} independently are selected from a functional group obtained by reaction with: an amino acid, a polypeptide, adenine, guanine, tyrosine, cytosine, uracil, biotin, ferrocene, ruthenocene, cyanuric chloride, or methacryloyl chloride;
Rₑ₁, Rₑ₂, and Rₑ₃, are independently selected from a hydrogen atom, a linear or branched alkyl group with up to 25 carbons, —(CH₂CH₂O)_{α}—(CH₂)_{β}OR_{g1}, —(CH₂CH₂O)_{α}, —(CH₂)_{β}NR_{g2}R_{g3}, —(CH₂CH₂O)_{α}—(CH₂)_{β}CONR_{g2}R_{g3}, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, aryl groups, fused aromatic rings, vinyl, allyl, 4-styryl, acroyl, methacroyl, acrylonitrile, isocyanate, isothiocyanate, epoxides, strained ring olefins, (—CH₂)_{γ}SiCl₃, (—CH₂)_{γ}Si(OCH₂CH₃)₃, or (—CH₂)_{γ}Si(OCH₃)₃;
R_{g1}, R_{g2}, and R_{g3} independently are selected from a hydrogen atom, a linear or branched alkyl group with up to 25 carbons, or a functional group obtained by reaction with: an amino acid, a polypeptide, adenine, guanine, tyrosine, cytosine, uracil, biotin, ferrocene, ruthenocene, cyanuric chloride, or methacryloyl chloride;
α is an integer from 0 to 10, inclusive;
β and γ independently are integers in a range from 1 to 25, inclusive;
and wherein the at least one photocleavable group is bonded to the chromophore moiety through at least one of Rₐ, R_{b}, R_{c}, R_{d}, Rₑ, R_{f}, R_{g}, Rₕ, Rᵢ, Rⱼ, Rₖ, Rₗ, or Rₘ;
   (b) the at least one photocleavable group has the formula:

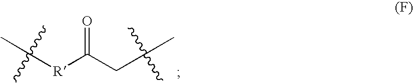

wherein R′ is a substituted or unsubstituted aryl or heteroaryl moiety, wherein any substituents on R′ are selected from alkyl, alkenyl, alkoxy, alkylcarboxy, hydroxy, or carboxy groups, wherein the at least one photocleavable group is bonded to the chromophore moiety through R′ or a substituent on R′, and wherein any substituent on R′ bonded to the chromophore moiety is selected from alkyl, alkenyl, alkoxy, or hydroxy groups; and
(c) the at least one protected group is selected from:

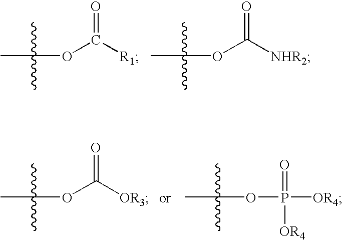

wherein R₁, R₂, R₃, and R₄ independently are selected from a substituted or unsubstituted alkyl, alkenyl, alkynyl, aryl, aralkyl, aralkenyl, aralkynyl, fused aryl, carbocyclic, carboxylalkyl-substituted alkyl, heterocycloalkyl, heterocycloalkyl-substituted alkyl, heteroaryl, heteroarylalkyl, heteroarylalkenyl, or heteroaralkynyl groups, wherein any substituents on R₁, R₂, R₃, and R₄ independently are selected from alkyl, alkenyl, alkynyl, alkoxy, acyl (alkanoyl), acyloxy, cyano, alkylcarboxy, chloro, bromo, aryl, cycloalkyl, aralkyl, aralkenyl, aralkynyl, hydroxy, nitro, amino, carboxy, amino acid, peptide, adenine, guanine, tyrosine, cytosine, uracil, biotin, ferrocene, ruthenocene, cyanuric chloride, methacryloyl chloride, glucose, mannose, galactose, gulose, allose, altrose, idose, talose, fructose, arabinose, xylose, sucrose, cellobiose, maltose, lactose, trehalose, gentiobiose, melibiose, raffinose, gentianose, adenosine, deoxyadenosine, guanosine, deoxyguanosine, cytidine, deoxycytidine, uridine, or deoxythymidine.

When the photocleavable group is described as bonded to the chromophore moiety through at least one of the chromophore R groups (Rₐ, R_{b}, R_{c}, etc.), it is understood that the normal rules of chemical valence apply. Thus, the bonding site on the photocleavable group typically is bonded to the chromophore R group by replacement of a hydrogen atom on that R group with the photocleavable group. As disclosed, the photocleavable group is bonded to the chromophore moiety through R′ or a substituent on R′ of the photocleavable group.

In another aspect, an intramolecular photodeprotection compound, comprising at least one chromophore moiety bonded to at least photocleavable group, the at least one photocleavable group bonded to at least one protected functional group, was produced. One such compound is

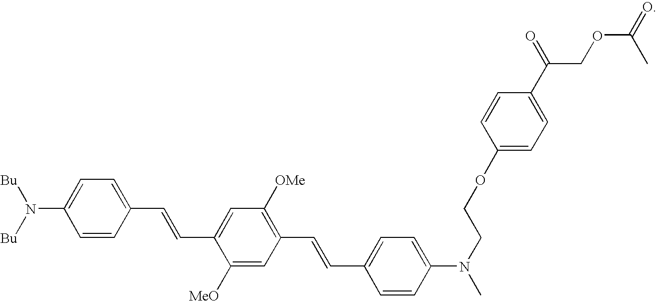

Additional compounds prepared herein include, but are not limited to,

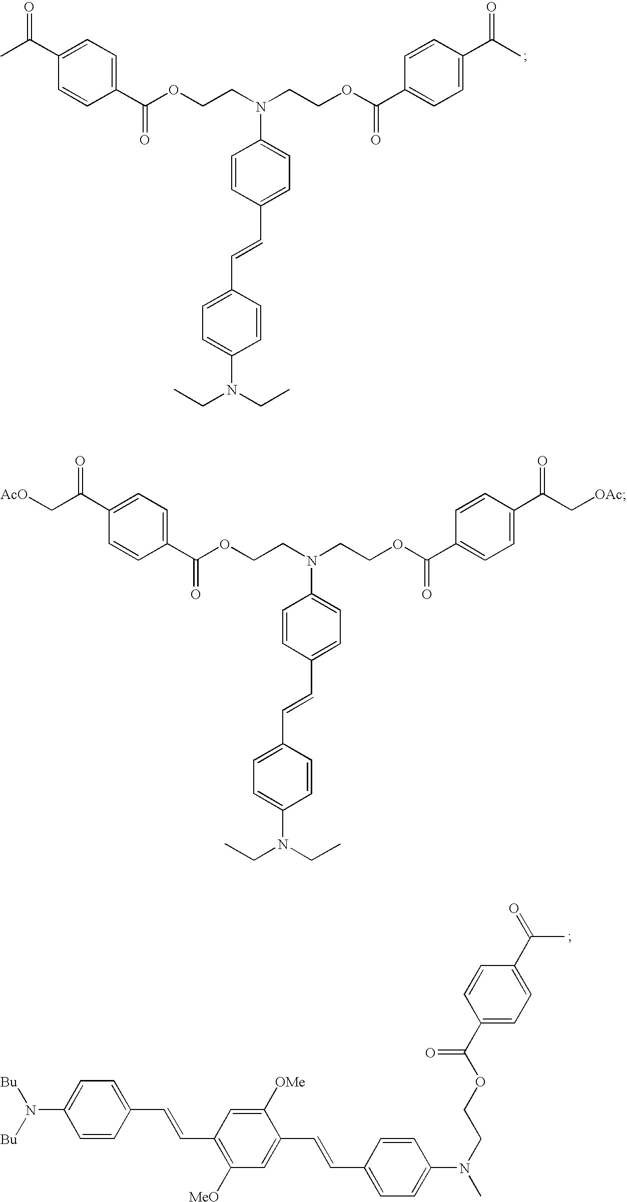

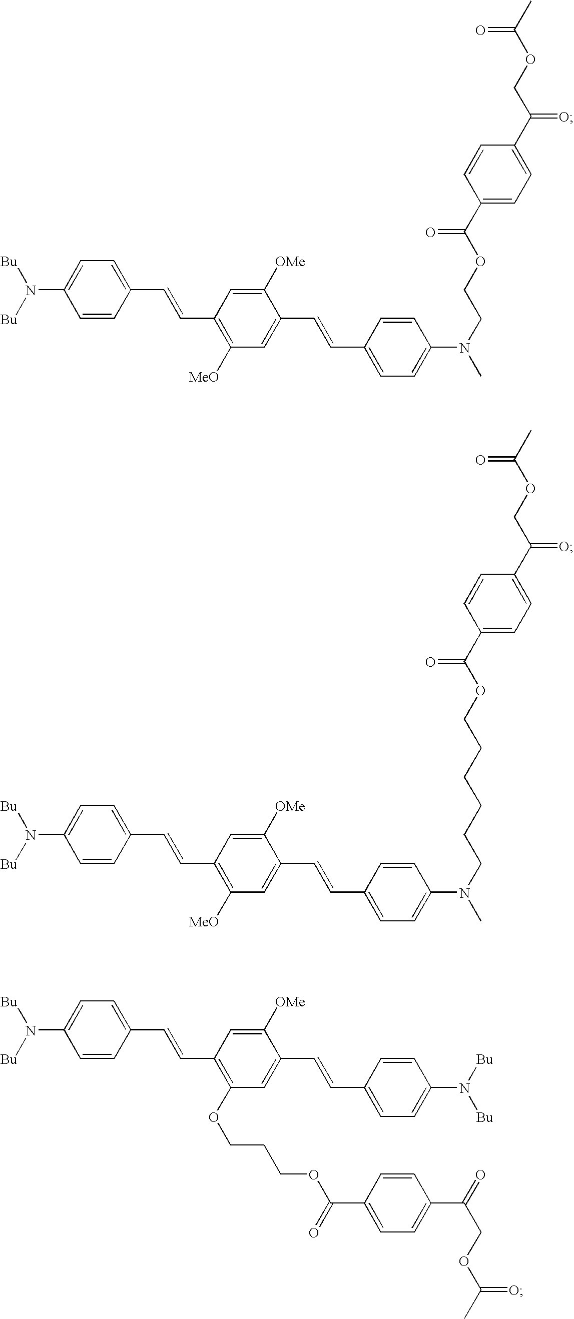

and the like.

In accordance with the present invention, intramolecular photodeprotection compounds comprising at least one chromophore moiety bonded to at least photocleavable group, the at least one photocleavable group bonded to at least one protected functional group, can be produced by any means known to those of skill in the art. In one aspect, Reaction Scheme B illustrates the synthesis of both a model compound and a protected acetic acid covalently linked to a two-photon bis-donor-stilbene chromophore:

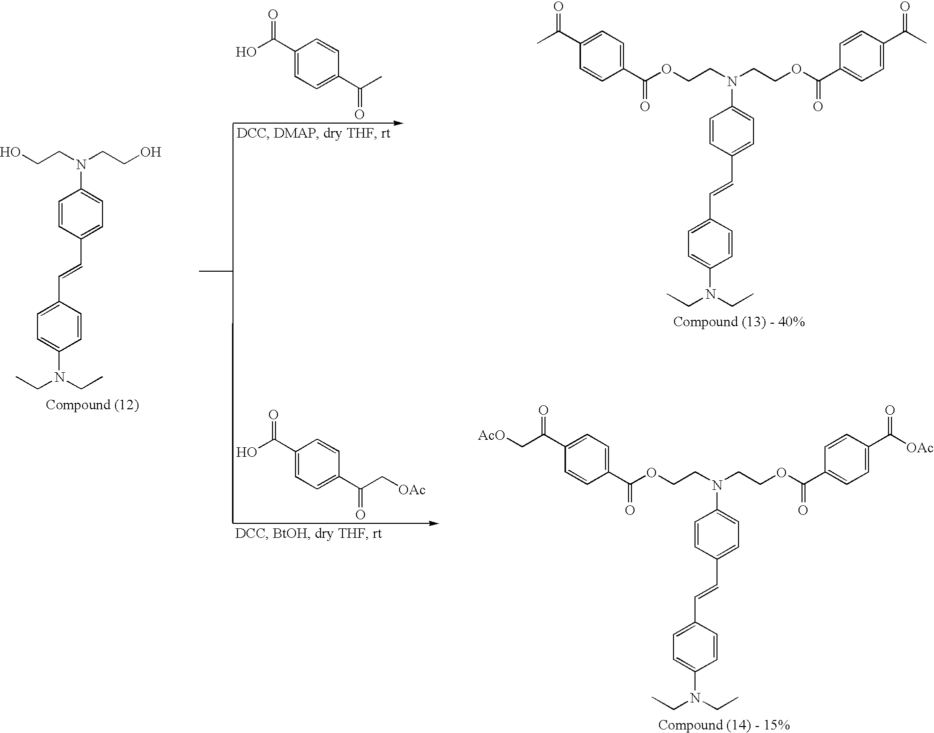

Non-limiting examples of the synthesis of Compound (13) and Compound (14) are illustrated in Examples 5 and 6, respectively.

Reaction Scheme C illustrates the synthesis of both a model compound and a protected acetic acid covalently linked to a two-photon chromophore:

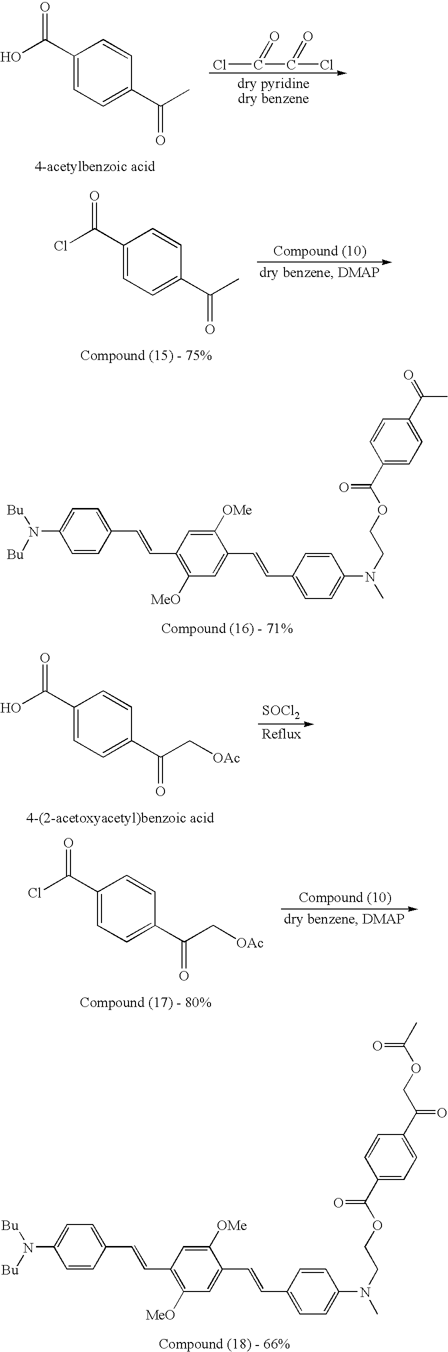

Non-limiting examples of the synthesis of Compound (16) and Compound (18) are illustrated in Examples 7 and 8, respectively.

Reaction Scheme D illustrates the synthesis of a compound having a protected acetic acid and a photocleavable group covalently linked to a two-photon chromophore.

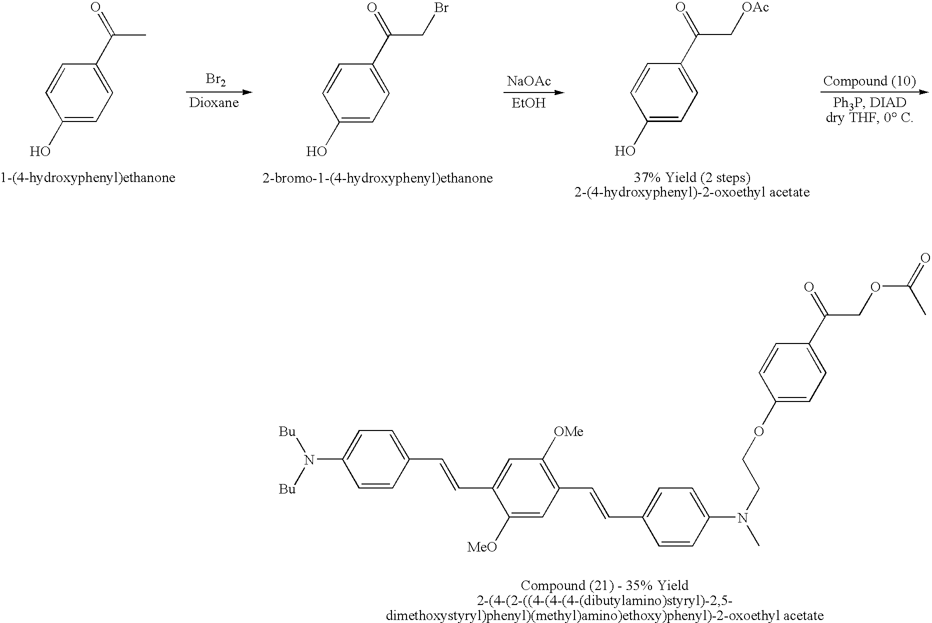

An intramolecular photodeprotection compound as described above can be used in a method for deprotecting a protected functional group. Such a method comprises:

(a) providing a compound, the compound comprising at least one chromophore moiety bonded to at least photocleavable group, the at least one photocleavable group bonded to at least one protected functional group;

(b) exposing the compound to radiation;

(c) converting the compound to a multi-photon electronically excited state upon simultaneous absorption of at least two photons of the radiation by the compound, wherein the sum of the energies of all of the absorbed photons is greater than or equal to the transition energy from a ground state of the compound to the multi-photon excited state and wherein the energy of each absorbed photon is less than the transition energy between the ground state and the lowest single-photon excited state of the compound and is less than the transition energy between the multi-photon excited state and the ground state;

thereby inducing an electron transfer from the chromophore moiety to the photocleavable group to cleave and deprotect the protected functional group.

### DEFINITIONS

The following definitions are set forth to illustrate and define the meaning and scope of the various terms used to describe the invention herein.

Generally, a donor is an atom or group of atoms with a low ionization potential that can be bonded to a π(pi)-conjugated bridge, and an acceptor is an atom or group of atoms with a high electron affinity that can be bonded to a π(pi)-conjugated bridge. A bridging group is a molecular fragment that connects two or more smaller chemical moieties or units; bridging groups can also contain donor groups and acceptor groups.

The term “leaving group” means a group capable of being displaced by a nucleophile in a chemical reaction, for example halo, nitrophenoxy, pentafluorophenoxy, alkyl sulfonates (e.g., methanesulfonate), aryl sulfonates, phosphates, sulfonic acid, sulfonic acid salts, and the like.

“Activating group” refers to those groups which, when attached to a particular functional group or reactive site, render that site more reactive toward covalent bond formation with a second functional group or reactive site. For example, the group of activating groups which can be used in the place of a hydroxyl group include —O(CO)Cl; —OCH₂Cl; —O(CO)OAr, where Ar is an aromatic group, preferably, a p-nitrophenyl group; —O(CO)(ONHS); and the like. The group of activating groups which are useful for a carboxylic acid include simple ester groups and anhydrides. The ester groups include alkyl, aryl and alkenyl esters and in particular such groups as 4-nitrophenyl, N-hydroxylsuccinimide and pentafluorophenol. Other activating groups are known to those of skill in the art.

“Alkyl” refers to a cyclic, branched, or straight chain saturated hydrocarbon radical, typically having from one to twenty carbon atoms, such as methyl, heptyl, —(CH₂)₂—, and adamantyl. Alkyl groups can either be unsubstituted or substituted with one or more substituents, e.g., halogen, alkoxy, acyloxy, amino, aryl, hydroxyl, mercapto, carboxy, benzyloxy, phenyl, benzyl, or other functionality which may be suitably blocked, if necessary for purposes of the invention, with a protecting group. When “alkyl” or “alkylene” is used to refer to a linking group or a spacer, it is taken to be a group having two available valences for covalent attachment, for example, —CH₂CH₂—, CH₂CH₂CH₂—, —CH₂CH₂CH(CH₃)CH₂— and —CH2(CH2)₂CH₂—. Preferred alkyl groups as substituents are those containing 1 to 10 carbon atoms, with those containing 1 to 6 carbon atoms being particularly preferred. Preferred alkyl or alkylene groups as linking groups are those containing 1 to 20 carbon atoms, with those containing 3 to 6 carbon atoms being particularly preferred.

“Alkoxy” refers to the group alkyl-O—.

The term “alkenyl” refers to an unsaturated hydrocarbon radical which contains at least one carbon-carbon double bond and includes straight chain, branched chain and cyclic radicals.

The term “alkynyl” refers to an unsaturated hydrocarbon radical which contains at least one carbon-carbon triple bond and includes straight chain, branched chain and cyclic radicals.

The term “aryl” or “Ar” refers to an aromatic monovalent carbocyclic radical having a single ring (e.g., phenyl) or two condensed or fused rings (e.g., naphthyl), or the like. Thus, Aryl” or “Ar” includes an aromatic substituent which may be multiple rings which are fused together, linked covalently, or linked to a common group such as an ethylene or methylene moiety. The aromatic rings may each contain heteroatoms, for example, phenyl, naphthyl, biphenyl, diphenylmethyl, 2,2-diphenyl-1-ethyl, thienyl, pyridyl and quinoxalyl. The aryl moieties may also be optionally substituted with halogen atoms, or other groups such as nitro, carboxyl, alkoxy, phenoxy and the like. Additionally, the aryl radicals may be attached to other moieties at any position on the aryl radical which would otherwise be occupied by a hydrogen atom (such as, for example, 2-pyridyl, 3-pyridyl and 4-pyridyl). Aryl groups optionally can be mono-, di-, or tri-substituted, independently, with alkyl, lower-alkyl, cycloalkyl, hydroxylower-alkyl, aminolower-alkyl, hydroxyl, thiol, amino, halo, nitro, lower-alkylthio, lower-alkoxy, mono-lower-alkylamino, di-lower-alkylamino, acyl, hydroxycarbonyl, lower-alkoxycarbonyl, hydroxysulfonyl, lower-alkoxysulfonyl, lower-alkylsulfonyl, lower-alkylsulfinyl, trifluoromethyl, cyano, tetrazoyl, carbamoyl, lower-alkylcarbamoyl, and di-lower-alkylcarbamoyl. Alternatively, two adjacent positions of the aromatic ring may be substituted with a methylenedioxy or ethylenedioxy group. Typically, electron-donating substituents are preferred. Representative fused polycyclic aromatic hydrocarbons include naphthalene, phenanthracene, anthracene, benzoanthracene, dibenzoanthracene, heptalene, acenaphthalene, acephenanthracene, triphenylene, pyrene, fluorene, phenalene, naphthacene, picene, perylene, pentaphenylene, pyranthrene, fullerenes (including C₆₀ and C₇₀), and the like. A representative vinylogously substituted derivative of an aromatic hydrocarbon is styrene. A more complete description of aromaticity and heteroaromaticity can be found in J. March, Advanced Organic Chemistry: Reactions, Mechanisms and Structure, Fourth edition, Wiley-Interscience, New York, 1992. Chapter 2.

“Aryloxy” refers to the group aryl-O— or heteroaryl-O—.

“Amino” or “amine group” refers to the group —NR′R″, where R′ and R″ are independently selected from the group consisting of hydrogen, alkyl, substituted alkyl, aryl, substituted aryl, aryl alkyl, substituted aryl alkyl, heteroaryl, and substituted heteroaryl. In a primary amino group, both R′ and R″ are hydrogen, whereas in a secondary amino group, either, but not both, R′ or R″ is hydrogen.

“Arylalkyl” or “aralkyl” refers to the groups R′—Ar and R-HetAr, where Ar is an aryl group, HetAr is a heteroaryl group, and R′ is straight-chain or branched-chain aliphatic group (for example, benzyl, phenylethyl, 3-(4-nitrophenyl)propyl, and the like). Preferred aryl groups include phenyl, 1-naphthyl, 2-naphthyl, biphenyl, phenylcarboxylphenyl (i.e., derived from benzophenone), and the like.

“Carboxy” or “carboxyl” refers to the group —R′(COOH) where R′ is alkyl, substituted alkyl, aryl, substituted aryl, aryl alkyl, substituted aryl alkyl, heterocyclic, heteroaryl, or substituted heteroaryl.

“Carboxyalkyl” refers to the group —(CO)—R′ where R′ is alkyl or substituted alkyl.

“Carboxyaryl” refers to the group —(CO)—R′ where R′ is aryl, heteroaryl, or substituted aryl or heteroaryl.

The term “heteroaromatic” refers to an aromatic monovalent mono- or poly-cyclic radical having at least one heteroatom within the ring, e.g., nitrogen, oxygen or sulfur, wherein the aromatic ring can optionally be mono-, di- or tri-substituted, independently, with alkyl, lower-alkyl, cycloalkyl, hydroxylower-alkyl, aminolower-alkyl, hydroxyl, thiol, amino, halo, nitro, lower-alkylthio, lower-alkoxy, mono-lower-alkylamino, di-lower-alkylamino, acyl, hydroxycarbonyl, lower-alkoxycarbonyl, hydroxysulfonyl, lower-alkoxysulfonyl, lower-alkylsulfonyl, lower-alkylsulfinyl, trifluoromethyl, cyano, tetrazoyl, carbamoyl, lower-alkylcarbamoyl, and di-lower-alkylcarbamoyl. For example, typical heteroaryl groups with one or more nitrogen atoms are tetrazoyl, pyridyl (e.g., 4-pyridyl, 3-pyridyl, 2-pyridyl), pyrrolyl (e.g., 2-pyrrolyl, 2-(N-alkyl)pyrrolyl), pyridazinyl, quinolyl (e.g. 2-quinolyl, 3-quinolyl etc.), imidazolyl, isoquinolyl, pyrazolyl, pyrazinyl, pyrimidinyl, pyridonyl or pyridazinonyl; typical oxygen heteroaryl radicals with an oxygen atom are 2-furyl, 3-furyl or benzofuranyl; typical sulfur heteroaryl radicals are thienyl, and benzothienyl; typical mixed heteroatom heteroaryl radicals are furazanyl and phenothiazinyl. Further the term also includes instances where a heteroatom within the ring has been oxidized, such as, for example, to form an N-oxide or sulfone.

The term “optionally substituted” refers to the presence or lack thereof of a substituent on the group being defined. When substitution is present the group may be mono-, di- or tri-substituted, independently, with alkyl, lower-alkyl, cycloalkyl, hydroxylower-alkyl, aminolower-alkyl, hydroxyl, thiol, amino, halo, nitro, lower-alkylthio, lower-alkoxy, mono-lower-alkylamino, di-lower-alkylamino, acyl, hydroxycarbonyl, lower-alkoxycarbonyl, hydroxysulfonyl, lower-alkoxysulfonyl, lower-alkylsulfonyl, lower-alkylsulfinyl, trifluoromethyl, cyano, tetrazoyl, carbamoyl, lower-alkylcarbamoyl, and di-lower-alkylcarbamoyl. Typically, electron-donating substituents such as alkyl, lower-alkyl, cycloalkyl, hydroxylower-alkyl, aminolower-alkyl, hydroxyl, thiol, amino, halo, lower-alkylthio, lower-alkoxy, mono-lower-alkylamino and di-lower-alkylamino are preferred.

The term “electron donating group” refers to a radical group that has a lesser affinity for electrons than a hydrogen atom would if it occupied the same position in the molecule. For example, typical electron donating groups are hydroxy, alkoxy (e.g. methoxy), amino, alkylamino and dialkylamino.

The term “amino acid” refers to the twenty naturally occurring amino acids, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine.

“Simultaneous” refers to two events that occur within the period of 10⁻¹⁴ sec.

A “two-photon absorption” is the process wherein a molecule absorbs two quanta of electromagnetic radiation.

A “multi-photon absorption” is the process wherein a molecule absorbs two or more quanta of electromagnetic radiation.

A “ligand” is a molecule that is recognized by a receptor. Examples of ligands which can be synthesized using the methods and compounds of this invention include, but are not restricted to, agonists and antagonists for cell membrane receptors, toxins and venoms, viral epitopes, hormones, opiates, steroids, peptides, enzyme substrates, cofactors, drugs, lectins, sugars, oligonucleotides, nucleic acids, oligosaccharides, and proteins.

A “receptor” is a molecule that has an affinity for a ligand. Receptors may be naturally-occurring or manmade molecules. They can be employed in their unaltered state or as aggregates with other species. Receptors may be attached, covalently or non-covalently, to a binding member, either directly or via a specific binding substance. Examples of receptors which can be employed by this invention include, but are not restricted to, antibodies, cell membrane receptors, monoclonal antibodies and antisera reactive with specific antigenic determinants, viruses, cells, drugs, polynucleotides, nucleic acids, peptides, cofactors, lectins, sugars, polysaccharides, cellular membranes, and organelles. Receptors are sometimes referred to in the art as anti-ligands. As the term receptors is used herein, no difference in meaning is intended. A “Ligand Receptor Pair” is formed when two molecules have combined through molecular recognition to form a complex.

Specific examples of receptors which can be investigated using ligands and libraries prepared using the methods and compounds of this invention include but are not restricted to:

a) Microorganism receptors: Determination of ligands that bind to microorganism receptors such as specific transport proteins or enzymes essential to survival of microorganisms would be a useful tool for discovering new classes of antibiotics. Of particular value would be antibiotics against opportunistic fungi, protozoa, and bacteria resistant to antibiotics in current use.

b) Enzymes: For instance, a receptor can comprise a binding site of an enzyme such as an enzyme responsible for cleaving a neurotransmitter; determination of ligands for this type of receptor to modulate the action of an enzyme that cleaves a neurotransmitter is useful in developing drugs that can be used in the treatment of disorders of neurotransmission.

c) Antibodies: For instance, the invention may be useful in investigating a receptor that comprises a ligand-binding site on an antibody molecule which combines with an epitope of an antigen of interest; determining a sequence that mimics an antigenic epitope may lead to the development of vaccines in which the immunogen is based on one or more of such sequences or lead to the development of related diagnostic agents or compounds useful in therapeutic treatments such as for autoimmune diseases (e.g., by blocking the binding of the “self” antibodies).

d) Nucleic Acids: Sequences of nucleic acids may be synthesized to establish DNA or RNA binding sequences that act as receptors for synthesized sequence.

e) Catalytic Polypeptides: Polymers, preferably antibodies, which are capable of promoting a chemical reaction involving the conversion of one or more reactants to one or more products. Such polypeptides generally include a binding site specific for at least one reactant or reaction intermediate and an active functionality proximate to the binding site, which functionality is capable of chemically modifying the bound reactant. Catalytic polypeptides and others are described in, for example, PCT Publication No. WO 90/05746, WO 90/05749, and WO 90/05785, which are incorporated herein by reference for all purposes.

f) Hormone receptors: Determination of the ligands which bind with high affinity to a receptor such as the receptors for insulin and growth hormone is useful in the development of, for example, an oral replacement of the daily injections which diabetics must take to relieve the symptoms of diabetes or a replacement for growth hormone. Other examples of hormone receptors include the vasoconstrictive hormone receptors; determination of ligands for these receptors may lead to the development of drugs to control blood pressure.

g) Opiate receptors: Determination of ligands which bind to the opiate receptors in the brain is useful in the development of less-addictive replacements for morphine and related drugs.

Specific examples of groups that can be used in this way include those disclosed in U.S. Pat. No. 6,147,205, and include, but are not limited to, groups of the type:

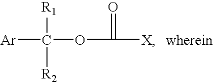

Ar is aryl or heteroaryl or is an optionally substituted fused polycyclic aryl or heteroaromatic group or a vinylogous derivative thereof;

R₁ and R₂ are independently H, optionally substituted alkyl, alkenyl or alkynyl, optionally substituted aryl or optionally substituted heteroaromatic, or a vinylogous derivative of the foregoing; and

X is a leaving group, a chemical fragment linked to Ar—C(R₁)(R₂)—O—C(O)— via a heteroatom, or a solid support; provided that when Ar is 1-pyrenyl and R₁═R₂═H, X is not linked to Ar—C(R₁)(R₂)—O—C(O)— via a nitrogen atom. Preferred embodiments are those in which Ar is a fused polycyclic aromatic hydrocarbon and in which the substituents on Ar, R₁, R₂ are electron donating groups. Particularly preferred protecting groups are the “PYMOC” protecting group, pyrenylmethyloxycarbonyl, where Ar=1-pyrenyl and R₁═R₂═H, and the “ANMOC” protecting group, anthracenylmethyloxycarbonyl, where Ar=anthracenyl and R₁═R₂═H.

### EXAMPLES

The invention is further illustrated by the following examples, which are not to be construed in any way as imposing limitations to the scope of this invention. Various other aspects, embodiments, modifications, and equivalents thereof which, after reading the description herein, may suggest themselves to one or ordinary skill in the art without departing from the spirit of the present invention or the scope of the appended claims.

NMR spectroscopy was performed using either a DRX-500 MHz spectrometer or a Varian Unity Plus 200 MHz or 300 MHz spectrometer. Mass spectrometry (MS) was performed by the MS Instrument Facility at the University of Arizona. All electrochemical experiments were conducted using a BAS Model 100B/W cyclic voltammetry (CV) unit.

The electrodes were a glassy-carbon working electrode, a platinum auxiliary wire, and a Ag/AgCl pseudo-reference electrode. The supporting electrolyte was 0.1 M tetrabutylammonium hexafluorosphonate in dichloromethane. Combustion experiments for elemental analysis were conducted by Desert Analytics of Tucson, Ariz. Silica gel (40-63 μm, from EMD Chemical, Inc.) was used to perform flash column chromatography. TLC was performed on pre-coated plates containing a fluorescent indicator (silica gel 60 F₂₅₄, from EMD Chemicals, Inc.), All reagents and solvents including dry solvents and anhydrous solvents in Acroseal bottles were purchased from readily available suppliers (e.g., Aldrich and Acros), and were used as received.

### Example 1

### Synthesis of a Compound with a Photocleavable Group Bonded to a Protected Group: Acetic acid 2-oxo-2-phenyl-ethyl ester (2-oxo-2-phenylethyl acetate)

The structure for acetic acid 2-oxo-2-phenyl-ethyl ester, also referred to as 2-oxo-2-phenylethyl acetate, is shown below as Compound (1):

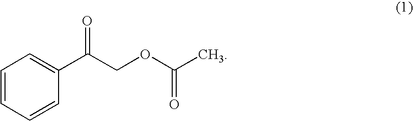

1,8-diazabicyclo[5,4,0]-undec-7-ene (DBU) (4.78 g, 4.69 mL, 26.0 mmol) and 2-bromoacetophone (5.20 g, 26.0 mmol) were added to a solution of acetic acid (2.04 g, 34 mmol) in benzene (80 mL) at room temperature under nitrogen. After 2 h, the reaction product was washed with 10% hydrogen chloride aqueous solution (3×80 mL) and then with saturated sodium carbonate (3×80 mL). The organic layers were dried over magnesium sulfate. 4.08 g of a white solid product, Compound (1), were obtained by recrystallization in diethyl ether. The yield was 88%. ¹H NMR (500 MHz, chloroform-d) δ 7.92 (d, J=8.0 Hz, 2H), 7.61 (d, J=8.0 Hz, 1H), 7.49 (d, J=8.0 Hz, 2H), 5.35 (m, 2H), 2.23 (s, 2H). The data were consistent with the literature data.

### Example 2

### Synthesis of a Compound with a Photocleavable Group Bonded to a Protected Group: 4-(2-acetoxy-acetyl)-benzoic acid 2-phenylamino-ethyl ester

The structure for 4-(2-acetoxy-acetyl)-benzoic acid 2-phenylamino-ethyl ester, also referred to as 2-(methyl(phenyl)amino)ethyl 4-(2-(ethanoyloxy)ethanoyl)benzoate, is shown below as Compound (2):

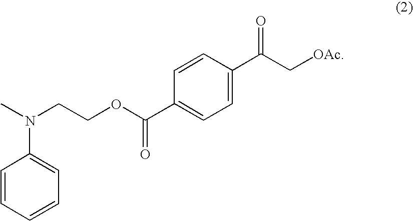

A reaction scheme for the synthesis of Compound (2), having a protected acetic acid group covalently bonded to a diakylaniline group, is shown below:

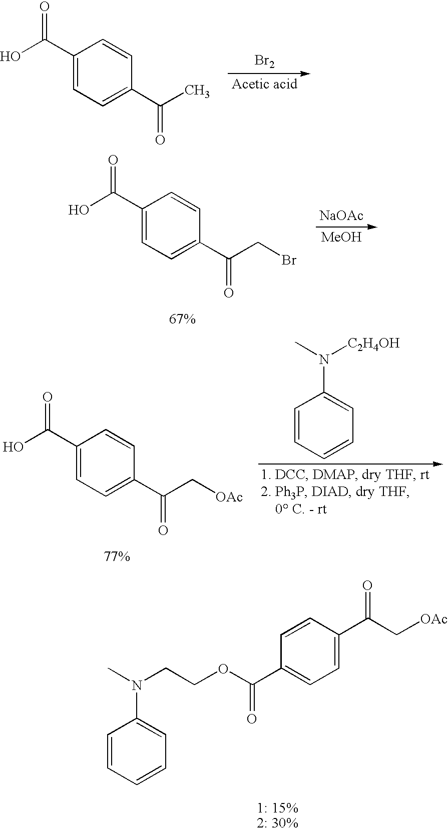

In the first step, 4-acetylbenzoic acid (10.76 g, 64.23 mmol) and acetic acid (200 mL) at room temperature were added to a 3-necked flask with a condenser. Bromine (3.3 mL) in methanol (35 mL) were added dropwise to the flask through a dripping funnel over 40 min at 35° C. The temperature was subsequently increased to 70° C. and maintained for 1 h. After cooling to room temperature, the reaction mixture was filtered through a fritted funnel, the solid was washed with acetic acid (2×40 mL) and cold diethyl ether (2×50 mL), and then dried overnight (about 8 hours). 10.47 g of a white solid, 4-(2-bromoacetyl)-benzoic acid, were obtained. The yield was 67%. ¹H NMR (300 MHz, acetone-d₆) δ 8.17 (s, 4H), 4.34 (s, 2H). The data were consistent with the literature data.

4-(2-acetoxyacetyl)-benzoic acid is produced in the next step of the above reaction scheme. 14.78 g of sodium acetate (180.2 mmol) were added to methanol (200 mL), and the mixture was stirred until a clear solution resulted. A solution 4-(2-bromoacetyl)-benzoic acid (3.12 g, 12.8 mmol, prepared above) in methanol (400 mL) was added dropwise though a dripping funnel over 4 h at room temperature to the sodium acetate/methanol solution. This resulting reaction mixture was stirred overnight (about 8 hours) at room temperature, followed by heating under reflux for 1 h. After cooling to room temperature, the reaction mixture was filtered to remove solids from the solution. After the solution was concentrated under reduced pressure at 50° C. to dryness, cold water and a 5% sulfuric acid solution were added until precipitation was complete. The resulting white precipitate was collected by filtration, washed with water (2×80 mL), and dried in air overnight (about 8 hours). 2.85 g of a white solid, 4-(2-acetoxyacetyl)-benzoic acid, were obtained. The yield was 77% yield. ¹H NMR (500 MHz, chloroform-d) δ 8.20 (d, J=8.0 Hz, 2H), 7.99 (d, J=8.5 Hz, 2H), 5.34 (s, 2H), 2.23 (s, 3H). ¹C NMR (125 MHz, chloroform-d) δ 191.8, 170.4, 169.1, 138.0, 133.4, 130.7, 127.8, 66.1, 20.5. The data were consistent with the literature data.

Compound (2), 4-(2-acetoxy-acetyl)-benzoic acid 2-phenylamino-ethyl ester, can be produced by two alternative methods. In the first method, 0.30 mL of 2-(methylphenylamino)ethanol (2.1 mmol) and 0.70 g of 4-(2-acetoxyacetyl)-benzoic acid (3.2 mmol, prepared above) in dry THF (15 mL) were charged to a vessel at room temperature. 40 mg of 4-dimethylaminopyridine (DMAP, 0.32 mmol) and 0.65 g of N,N′ dicyclohexylcarbodimine (DCC, 3.2 mmol) were added to the vessel. The reaction mixture was stirred for 21 h. The insoluble solid was removed by filtration, and the filtrate was washed with saturated ammonia chloride and water. The combined aqueous layer was extracted with diethyl ether. The combined organic layers were washed with water, and dried over magnesium sulfate. Separation by column chromatography (hexane:ethyl acetate at 4:1) gave a mixture of 0.29 g (39% total yield) of reactant 2-(methylphenylamino)ethanol, 0.08 g (12% yield) of reaction by-product, acetic acid 2-(methyl-phenyl-amino)-ethyl ester. 0.11 g of the desired reaction product, Compound (2), were obtained by recrystallization in methanol. The yield was 15%.

In the second method, 0.50 mL of 2-(methylphenylamino)ethanol (0.53 g, 3.5 mmol), and 0.59 g of 4-(2-acetoxyacetyl)-benzoic acid (3.6 mmol, prepared above) and 0.95 g of triphenyl-phosphine (3.6 mmol) in dry THF (20 mL) were charged to a vessel at 0° C. 0.80 mL of diisopropyl azocarboxylate (DIAD, 0.84 g, 3.9 mmol) were added dropwise to the vessel. The reaction mixture was allowed to reach room temperature slowly overnight (about 8 hours), then quenched with a saturated sodium chloride solution. The aqueous layer was extracted with dichloromethane. The organic layers were dried over magnesium sulfate. Separation by column chromatography (hexane:ethyl acetate at 8:1) gave a mixture including 0.29 g (66% total yield) of reactant 2-(methylphenylamino)ethanol. 0.28 g of the desired reaction product, Compound (2), were obtained by recrystallization in methanol. The yield was 30%. ¹H NMR (500 MHz, chloroform-d) δ 8.04 (d, J=8.0 Hz, 2H), 7.93 (d, J=8.5 Hz, 2H), 7.24 (dd, J=9.0 Hz, J=7.5 Hz, 2H), 6.79 (d, J=8.0 Hz, 2H), 6.73 (t, J=7.5 Hz, 1H), 5.33 (s, 2H), 4.53 (t, J=5.5 Hz, 2H), 3.77 (t, J=6.0 Hz, 2H), 3.04 (s, 3H), 2.23 (s, 3H). ¹³C NMR (125 MHz, chloroform-d) δ 191.9, 170.3, 165.5, 149.0, 137.4, 134.4, 130.1, 129.3, 127.7, 116.9, 112.4, 66.1, 62.7, 51.1, 38.6, 20.5. HRMS (FAB+) m/z: Calcd. for C₂₀H₂₁NO₅(M⁺) 355.1420, Found 355.1438. Anal. Calcd. for C₄₀H₄₂N₂O₆: C, 67.59; H, 5.96; N, 3.49. Found: C, 67.60; H, 5.92; N, 3.91.

### Example 3

### Synthesis of a Compound Containing a Photocleavable Group: 2-(methyl(phenyl)amino)ethyl 4-acetylbenzoate

The structure for 2-(methyl(phenyl)amino)ethyl 4-acetylbenzoate, is shown below as Compound (3):

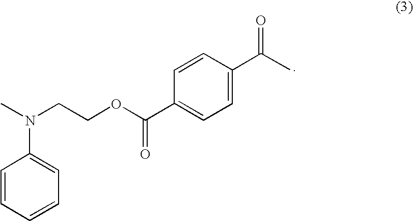

A reaction scheme for the synthesis of Compound (3) is shown below:

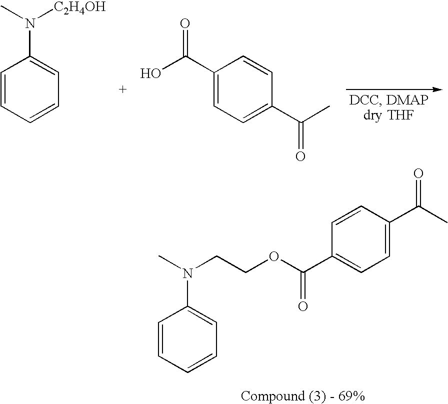

The method described in last step of the synthesis in Example 2, using DCC, DMAP, and dry THF was employed, where 4-acetylbenzoic acid was used instead of 4-(2-acetoxyacetyl)-benzoic acid. The yield of Compound (3), 2-(methyl(phenyl)amino)ethyl 4-acetylbenzoate, was 69%.

### Example 4

### Synthesis of a Chromophore Compound with a Hydroxyl Functionality for Covalent Attachment to Photocleavable Group: 2-{[4-(2-{4-[2-(4-dibutylamino-phenyl)-vinyl]-2,5-dimethoxy-phenyl}-vinyl)-phenyl]methyl-amino}-ethanol

The structure for 2-{[4-(2-{4-[2-(4-dibutylamino-phenyl)-vinyl]-2,5-dimethoxy-phenyl}-vinyl)-phenyl]methyl-amino}-ethanol, also referred to as 2-((4-(4-(4-(dibutylamino)styryl)-2,5-dimethoxystyryl)phenyl)(methyl)amino)ethanol, is shown below as Compound (10):

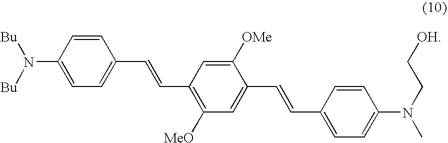

A reaction scheme for the synthesis of chromophore Compound (10) is shown below:

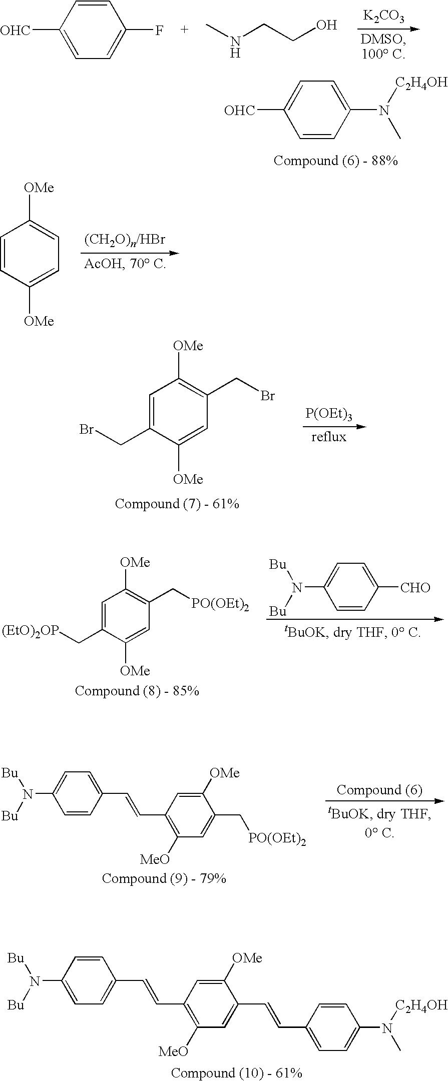

In a preliminary step, Compound (6) above, 4-[(2-hydroxy-ethyl)-methyl-amino]-benzaldehyde, is synthesized. To a solution of 23 mL of 2-(methylamino)ethanol (0.28 mol) and 20.11 g of potassium carbonate (0.15 mmol) in DMSO (100 mL), 10 mL of 4-fluoro-benzaldehyde (93.2 mmol) were added. The reaction mixture was heated at 100° C. for 68 h. After cooling to room temperature, the reaction product was poured into ice water (500 mL) and then extracted with dichloromethane. The combined organic layers were dried over magnesium sulfate. Subsequent removal of solvent by evaporation resulted in 14.70 g of Compound (6). The yield was 88%. ¹H NMR (500 MHz, chloroform-d) δ 9.65 (s, 1H), 7.66 (d, J=9.0 Hz, 2H), 6.72 (d, J=9.0 Hz, 2H), 3.84 (t, J=6.0 Hz, 2H), 3.59 (t, J=6.0 Hz, 2H), 3.09 (s, 3H).

In the first step, 70 mL of hydrogen bromide (30% wt. in acetic acid) were added to a suspension of 15.82 g of 1,4-dimethoxybenzene (0.11 mol) and 7.46 g of paraformaldehyde (0.24 mol) in acetic acid (220 mL). The reaction mixture was heated to 70° C. for 4 h. After cooling to room temperature, the reaction mixture was poured into water (1500 mL), and the crude reaction product was collected by filtration. Trituration with hot methanol resulted in 22.32 g of Compound (7), 1,4-bis(bromomethyl)-2,5-dimethoxybenzene. The yield was 61%. ¹H NMR (500 MHz, chloroform-d) δ 6.85 (s, 2H), 4.51 (s, 4H), 3.85 (s, 6H).

Next, 11.99 g of Compound (7) (12.22 mmol, prepared above) is mixed with 50 mL of triethyl phosphate and was refluxed for 18 h. After cooling to room temperature, the reaction mixtures was placed in a freezer for 2 h. The desired product crystallized from the triethyl phosphite. The excess triethyl phosphite was decanted, and the residue was washed with hexane (3×80 mL). Subsequent filtration and drying resulted in 13.7 g of Compound (8), [4-(diethoxy-phosphorylmethyl)-2,5-dimethoxy-benzyl]-phosphonic acid diethyl ester. The yield was 85% yield. ¹H NMR (500 MHz, chloroform-d) δ 6.86 (d, J=1.5 Hz, 2H), 3.98 (dq, J=7.5 Hz, 8H), 3.75 (s, 6H), 3.20 (s, 2H), 3.16 (s, 2H), 1.20 (t, J=7.5 Hz, 12H).

In the next step, 6.5 mL of potassium tert-butoxide (6.0 mmol, 1.0 M in 2-methyl-2-propanol) were added to a solution of 0.98 g of 4-(dimethylamino)benzaldehyde (1 mL, 4.2 mmol) and 2.81 g of Compound (8) (6.41 mmol, prepared above) in dry THF (80 mL) at 0° C. After stirring at 0° C. for 2 h, the reaction mixture was quenched by adding 50 mL of water. THF was removed by evaporation, and the aqueous layer was extracted with dichloromethane until the color of the aqueous layer was only slight yellow or colorless. The combined organic layers were washed with water, and dried over magnesium sulfate. Column chromatography (ethyl acetate) resulted in 1.52 g of Compound (9), {-4-[2-(4-dibutylamino-phenyl)-vinyl]-2,5-dimethoxy-benzyl}phosphonic acid diethyl ester. The yield was 79%. ¹H NMR (500 MHz, chloroform-d) δ 7.39 (d, J=8.5 Hz, 2H), 7.20 (d, J=16.5 Hz, 1H), 7.07 (s, 1H), 6.98 (d, J=16.0 Hz, 1H), 6.90 (d, J=2.5 Hz, 1H), 6.61 (d, J=9.0 Hz, 2H), 4.04 (dq, J=7.0 Hz, 4H), 3.86 (s, 3H), 3.83 (s, 3H), 3.21-3.29 (m, 6H, 2NCH₂), 1.58 (quintet, J=8.0 Hz, 4H), 1.36 (sextet, J=7.5 Hz, 4H), 1.26 (t, J=7.5 Hz, 6H), 0.95 (t, J=7.5 Hz, 6H).

In the last step, 2.4 mL of potassium tert-butoxide (2.4 mmol, 1.0 M in 2-methyl-2-propanol) were added to a solution of 0.37 g of Compound (9) (0.80 mmol, prepared above) and 0.15 g of Compound (6) (0.80 mmol, prepared above) in dry THF (15 mL) at 0° C. After stirring at 0° C. for 4 h, then at room temperature for 3 h, the reaction mixture was quenched by adding of water. THF was removed by evaporation, and the aqueous layer was extracted with ether and dichloromethane until the color of the aqueous layer was only slight yellow or colorless. The combined organic layers were washed with water, and dried over magnesium sulfate. Column chromatography (hexane:ethyl acetate at 2:1) resulted in 0.26 g of Compound (10), 2-{[4-(2-{4-[2-(4-dibutylamino-phenyl)-vinyl]-2,5-dimethoxy-phenyl}-vinyl)-phenyl]-methyl-amino}-ethanol. The yield was 61%. ¹H NMR (500 MHz, acetone-d₆) δ 7.38 (d, J=9.0 Hz, 2H), 7.37 (d, J=9.0 Hz, 2H), 7.25 (d, J=16.5 Hz, 1H), 7.24 (s, 2H), 7.23 (d, J=16.5 Hz, 1H), 7.14 (d, J=16.5 Hz, 1H), 7.13 (d, J=16.5 Hz, 1H), 6.74 (d, J=9.0 Hz, 2H), 6.68 (d, J=9.0 Hz, 2H), 3.90 (s, 6H), 3.73 (t, J=6.5 Hz, 2H), 3.50 (t, J=6.5 Hz, 2H), 3.35 (t, J=7.5 Hz, 4H), 3.02 (s, 3H), 1.59 (quintet, J=7.5 Hz, 4H), 1.38 (six, J=7.5 Hz, 4H), 0.95 (t, J=7.5 Hz, 6H). ¹³C NMR (125 MHz, acetone-d₆) δ 152.1, 152.0, 149.9, 148.7, 129.5, 129.4, 128.5 127.3, 127.1, 126.7, 126.1, 118.9, 118.6, 112.8, 112.6, 109.2, 109.1, 59.9, 56.5, 56.5, 55.4, 51.2, 39.2, 20.9, 14.3. HRMS (FAB+) m/z: Calcd. for C₃₆H₄₆N₂O₃(M⁺) 542.3508, Found 542.3525. Anal. Calcd. for C₃₅H₄₆N₂O₃: C, 77.45; H, 8.54; N, 5.16. Found: C, 77.06; H, 8.66; N, 5.16.

### Example 5

### Synthesis of a Compound Having at Least One Chromophore Moiety Bonded to at Least One Photocleavable Group: (E)-2,2′-(4-(4-(diethylamino)styryl)phenylazanediyl)bis(ethane-2,1-diyl)bis(4-acetylbenzoate)

The structure for (E)-2,2′-(4-(4-(diethylamino)styryl)phenylazanediyl)bis(ethane-2,1-diyl)bis(4-acetylbenzoate) is shown below as Compound (13):

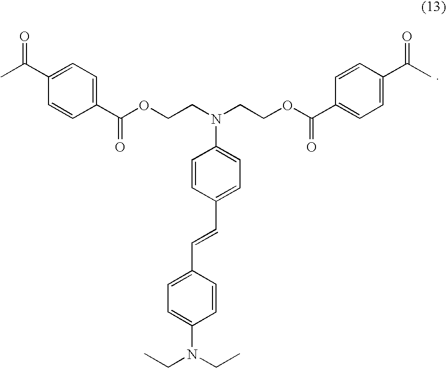

To a solution of 0.3 g of Compound (12), 2-[{4-[2-(4-Diethylamino-phenyl)-vinyl]-phenyl}-(2-hydroxy-ethyl)-amino]-ethanol (0.85 mmol), and 0.43 g of 4-acetylbenzoic acid (2.6 mmol) in dry THF (25 mL), 40 mg of DMAP (0.28 mmol) and 0.54 g of DCC (2.6 mmol) were added at room temperature. The reaction mixture was stirred for 3 h. After the insoluble solid was removed by filtration, the remaining solution was washed with saturated ammonia chloride and water. The combined aqueous layer was then extracted with diethyl ether. The combined organic layers were washed with water and dried over magnesium sulfate. Purification by column chromatography (hexane:ethyl acetate at 6:1 and then at 4:1) resulted in 0.17 g of Compound (13). The yield was 40%. ¹H NMR (500 MHz, acetonitrile-d₃) δ 7.99 (d, J=8.5 Hz, 4H), 7.94 (d, J=8.5 Hz, 4H), 7.34 (d, J=9.0 Hz, 2H), 7.31 (d, J=9.0 Hz, 2H), 6.80 (d, J=8.5 Hz, 2H), 6.84 (s, 1H), 6.83 (s, 1H), 6.67 (d, J=8.5 Hz, 2H), 4.51 (t, J=6.0 Hz, 4H), 3.85 (t, J=6.0 Hz, 2H), 3.37 (q, J=7.0 Hz, 4H), 2.55 (s, 6H), 1.23 (t, J=6.0 Hz, 6H). ¹³C NMR (125 MHz, acetone-d₆) δ 197.56, 166.13, 147.85, 147.72, 141.36, 134.50, 130.46, 129.02, 128.35, 128.05, 127.91, 126.35, 125.90, 124.37, 113.72, 112.66, 63.61, 50.30, 44.86, 26.92, 12.92. HRMS (FAB+) m/z: Calcd. for C₄₀H₄₂N₂O₆ (M⁺) 647.3121, Found 647.3137. Anal. Calcd. for C₄₀H₄₂N₂O₆: C, 74.28; H, 6.55; N, 4.33. Found: C, 73.89; H, 6.69; N, 4.27.

### Example 6

### Synthesis of a Compound Having at Least One Chromophore Moiety Bonded to at Least One Photocleavable which is Bonded to at Least One Protected Functional Group: (E)-2,2′-(4-(4-(diethylamino)styryl)phenylazanediyl)bis(ethane-2,1-diyl)bis(4-(2-acetoxyacetyl-benzoate)

The structure for (E)-2,2′-(4-(4-(diethylamino)styryl)phenylazanediyl)bis(ethane-2,1-diyl)bis(4-(2-acetoxyacetyl)benzoate) is shown below as Compound (14):

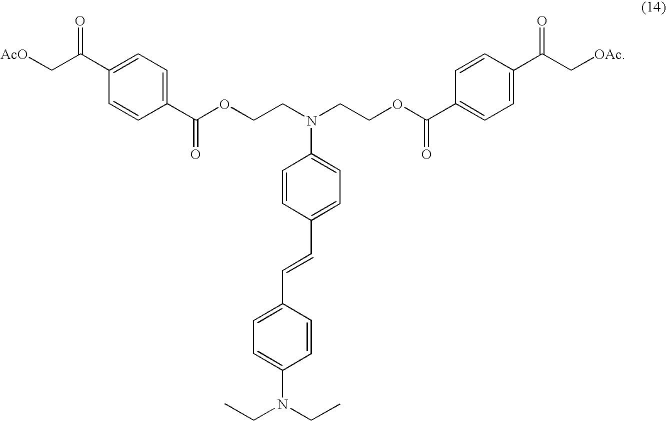

To a solution 0.12 g of Compound (12), 2-[{4-[2-(4-Diethylamino-phenyl)-vinyl]-phenyl}-(2-hydroxy-ethyl)-amino]-ethanol (0.35 mmol), and 0.23 g of 4-(2-acetoxyacetyl)benzoic acid (1.1 mmol) in dry THF (15 mL), 40 mg of DMAP (0.32 mmol) and 0.22 g of DCC (1.1 mmol) were added at room temperature. The reaction mixture was stirred at room temperature for 16 h. After the insoluble solid was removed by filtration, the filtrate was washed with saturated ammonia chloride and water. The combined aqueous layer was then extracted with diethyl ether. The combined organic layers were washed with water and dried over magnesium sulfate. Separation by column chromatography (hexane:ethyl acetate at 2:1) resulted in 0.04 g of Compound (14). The yield was 15% yield. ¹H NMR (500 MHz, acetone-d₆) δ 8.09 (d, J=8.5 Hz, 4H), 8.03 (d, J=8.5 Hz, 4H), 7.38 (d, J=9.0 Hz, 2H), 7.33 (d, J=9.0 Hz, 2H), 6.97 (d, J=9.0 Hz, 2H), 6.86 (d, J=8.5 Hz, 2H), 6.67 (d, J=8.5 Hz, 2H), 5.42 (s, 1H), 4.60 (t, J=6.0 Hz, 4H), 3.97 (t, J=6.0 Hz, 2H), 3.39 (q, J=7.0 Hz, 4H), 2.12 (s, 6H), 1.14 (t, J=7.0 Hz, 6H). ¹³C NMR (125 MHz, acetone-d₆) δ 192.9, 170.4, 165.9, 147.8, 138.6, 135.1, 130.6, 128.7, 128.1, 127.9, 125.9, 113.8, 112.7, 67.1, 63.7, 50.4, 44.9, 20.4, 12.9. HRMS (FAB+) m/z: Calcd. for C₄₄H₄₇N₂O₁₀ (MH⁺) 736.3231, Found 736.3240.

### Example 7

### Synthesis of a Compound Having at Least One Chromophore Moiety Bonded to at Least One Photocleavable Group: 4-acetyl-benzoic acid 2-{[4-(2-{4-[2-(4-dibutylamino-phenyl)-vinyl]-2,5-dimethoxy-phenyl}-vinyl)-phenyl]-methyl-amino}-ethyl ester

The structure for 4-acetyl-benzoic acid 2-{[4-(2-{4-[2-(4-dibutylamino-phenyl)-vinyl]-2,5-dimethoxy-phenyl}-vinyl)-phenyl]-methyl-amino}-ethyl ester, also referred to as 2-((4-(4-(4-(dibutylamino)styryl)-2,5-dimethoxystyryl)phenyl)methyl)amino)ethyl 4-acetylbenzoate, is shown below as Compound (16):

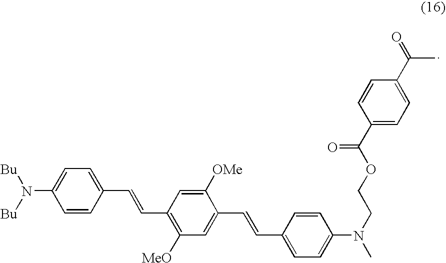

As illustrated in Reaction Scheme C above, Compound (15), 4-acetylbenzoyl chloride is first produced. To a mixture of 1.94 g of 4-acetylbenzoic acid (11.6 mmol) in 1 mL of dry pyridine and 30 mL dry benzene, 6 mL of oxalyl chloride (68 mmol) were added dropwise at room temperature. A vigorous reaction occurred upon the addition of first few mL of oxalyl chloride. The reaction was stirred at room temperature for 30 min, then heated to 80° C. for 40 min, and finally to 100° C. for 40 min. After subsequent cooling to room temperature, the reaction mixture was filtered and the filtrate was concentrated by evaporation. Subsequent recrystallization in hexane resulted in 1.57 g of Compound (15). The yield was 75%. ¹H NMR (500 MHz, chloroform-d) δ 8.21 (d, J=8.0 Hz, 2H), 8.06 (d, J=8.5 Hz, 2H), 2.67 (s, 3H). The data were consistent with the literature data.

Compound (15), as prepared above, and Compound (10), as prepared in Example 4, are used as reactants to produce Compound (16). A mixture of 98 mg of Compound (10) (0.18 mmol), 41 mg of Compound (15) (0.22 mmol), and 69 mg of DMAP (0.56 mmol) in 30 mL of dry benzene was heated to reflux for 2 h. After cooling to room temperature, the reaction mixture was quenched by adding a 50 mL of a saturated sodium chloride solution, followed by extraction with ethyl acetate (3×30 mL). The combined organic layers were dried over magnesium sulfate. Isolation by column chromatography (hexane:ethyl acetate at 4:1) resulted in 88 mg of Compound (16). The yield was 71%. ¹H NMR (500 MHz, acetone-d₆) δ 8.01 (s, 4H), 7.40 (d, J=8.5 Hz, 2H), 7.37 (d, J=8.0 Hz, 2H), 7.27 (d, J=16.5 Hz, 1H), 7.25 (d, J=16.5 Hz, 1H), 7.24 (s, 2H), 7.15 (d, J=16.5 Hz, 1H), 7.12 (d, J=16.5 Hz, 1H), 6.82 (d, J=9.0 Hz, 2H), 6.67 (d, J=9.0 Hz, 2H), 4.53 (t, J=5.5 Hz, 2H), 3.90 (s, 6H), 3.84 (t, J=7.5 Hz, 2H), 3.33 (t, J=7.5 Hz, 4H), 3.07 (s, 3H), 2.57 (s, 3H), 1.58 (quintet, J=7.5 Hz, 4H), 1.36 (sextet, J=7.5 Hz, 4H), 0.95 (t, J=7.5 Hz, 6H). ¹³C NMR (125 MHz, acetone-d₆) δ 197.52, 166.05, 152.10, 152.01, 149.75, 148.68, 141.27, 134.46, 130.43, 129.57, 129.26, 128.96, 128.48, 128.37, 127.43, 126.97, 126.09, 119.49, 118.61, 113.27, 112.60, 109.31, 109.13, 63.38, 56.55, 56.53, 51.45, 51.22, 38.75, 30.25, 26.89, 20.86, 14.27. HRMS (FAB+) m/z: Calcd for C₄₄H₅₂N₂O₆(M⁺) 688.3876, Found 688.3893. Anal. Calcd. for C₄₄H₅₂N₂O₆: C, 76.71; H, 7.61; N, 4.07. Found: C, 76.63; H, 7.76; N, 4.11.

### Example 8

### Synthesis of a Compound Having at Least One Chromophore Moiety Bonded to at Least One Photocleavable which is Bonded to at Least One Protected Functional Group: 4-(2-acetoxyacetyl)-benzoic acid 2-{[4-(2-{4-[2-(4-dibutylamino-phenyl)-vinyl]-2,5-dimethoxy-phenyl}-vinyl)-phenyl]methyl-amino}-ethyl ester

The structure for 4-(2-acetoxyacetyl)-benzoic acid 2-{[4-(2-{4-[2-(4-dibutylamino-phenyl)-vinyl]-2,5-dimethoxy-phenyl}-vinyl)-phenyl]-methyl-amino}-ethyl ester, also referred to as 2-((4-(4-(4-(dibutylamino)styryl)-2,5-dimethoxystyryl)phenyl)(methyl)amino)ethyl 4-(2-acetoxyacetyl)benzoate, is shown below as Compound (18):

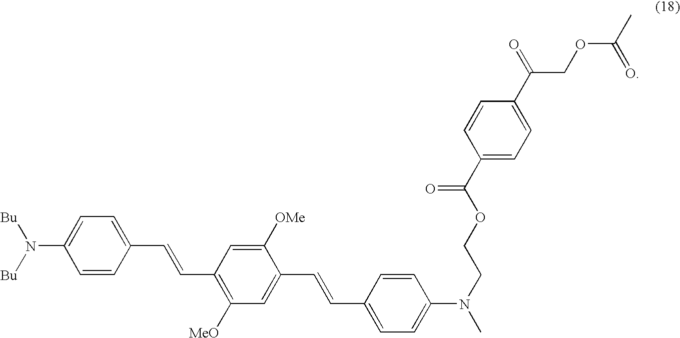

As illustrated in Reaction Scheme C above, Compound (17), acetic acid 2-(4-chlorocarbonyl-phenyl)-2-oxo-ethyl ester or 2-(4-(chlorocarbonyl)phenyl)-2-oxoethyl acetate, is first produced. A solution of 3.27 g of 4-(2-acetoxyacetyl)benzoic acid (14.7 mmol) in 25 mL of thionyl chloride was heated to reflux for 17 h. The thionyl chloride was removed by distillation and decomposed by adding a saturated potassium hydroxide aqueous solution. The residue was washed with water to remove residual thionyl chloride. Filtration resulted in 1.98 g of Compound (17). The yield 80%. ¹H NMR (500 MHz, chloroform-d) δ 8.21 (d, J=8.5 Hz, 2H), 8.00 (d, J=8.5 Hz, 2H), 5.32 (s, 2H), 2.22 (s, 3H). ¹C NMR (125 MHz, chloroform-d) δ 191.5, 170.3, 167.7, 138.9, 137.1, 131.6, 128.1, 66.1, 20.5. The data were consistent with the literature data.

Compound (17), as prepared above, and Compound (10), as prepared in Example 4, are used as reactants to produce Compound (18). A mixture of 0.21 g of Compound (10) (0.39 mmol), 0.28 mg of Compound (17) (1.2 mmol), and 0.15 g of DMAP (1.2 mmol) in 20 mL of dry benzene was heated to reflux for 2 h. After cooling to room temperature, the reaction mixture was quenched by adding a 50 mL of a saturated sodium chloride solution, followed by extraction with ethyl acetate (3×30 mL). The combined organic layers were dried over magnesium sulfate. Isolation by column chromatography (hexane:ethyl acetate at 8:1 and then 4:1) resulted in 0.19 mg of Compound (18). The yield was 66%. ¹H NMR (500 MHz, acetone-d₆) δ 8.03 (s, 4H), 7.40 (d, J=8.0 Hz, 2H), 7.37 (d, J=9.0 Hz, 2H), 7.27 (d, J=16.5 Hz, 1H), 7.25 (s, 1H), 7.24 (s, 1H), 7.23 (d, J=16.5 Hz, 1H), 7.15 (d, J=16.5 Hz, 1H), 7.14 (d, J=16.5 Hz, 1H), 6.84 (d, J=8.5 Hz, 2H), 6.67 (d, J=9.0 Hz, 2H), 5.40 (s, 2H), 4.55 (t, J=5.5 Hz, 2H), 3.90-3.86 (m, 8H), 3.34 (t, J=8.0 Hz, 4H), 3.08 (s, 3H), 2.11 (s, 3H), 1.58 (quintet, J=7.5 Hz, 4H), 1.37 (sextet, J=7.5 Hz, 4H), 0.95 (t, J=7.5 Hz, 6H). ¹³C NMR (125 MHz, acetone-d₆) δ 192.9, 170.4, 165.9, 152.1, 152.00, 149.8, 148.7, 138.5, 135.11, 130.6, 129.6, 129.3, 128.6, 128.5, 128.4, 127.5, 127.4, 126.9, 126.1, 119.5, 118.6, 113.3, 112.6, 109.30, 109.1, 67.1, 63.5, 56.5, 56.5, 38.7, 20.9, 20.3, 14.3. HRMS (FAB+) m/z: Calcd for C₄₆H₅₅N₂O₇ (MH⁺) 747.4009, Found 747.4001. Anal. Calcd. for C₄₆H₅₄N₂O₇: C, 73.97; H, 7.29; N, 3.75. Found: C, 73.61; H, 7.19; N, 3.59.

### Example 9

### Synthesis of a Compound Having at Least One Chromophore Moiety Bonded to at Least One Photocleavable which is Bonded to at Least One Protected Functional Group: 4-(2-acetoxyacetyl)-benzoic acid 6-{[4-(2-{4-[2-(4-dibutylamino-phenyl)-vinyl]-2,5-dimethoxy-phenyl}-vinyl)-phenyl]-methyl-amino}-hexyl ester

The structure for 4-(2-acetoxyacetyl)-benzoic acid 6-{[4-(2-{4-[2-(4-dibutylamino-phenyl)-vinyl]-2,5-dimethoxy-phenyl}-vinyl)-phenyl]methyl-amino}-hexyl ester, also referred to as 6-((4-(4-(4-(dibutylamino)styryl)-2,5-dimethoxystyryl)phenyl)(methyl)amino)hexyl 4-(2-acetoxyacetyl)benzoate, is shown below as Compound (20):

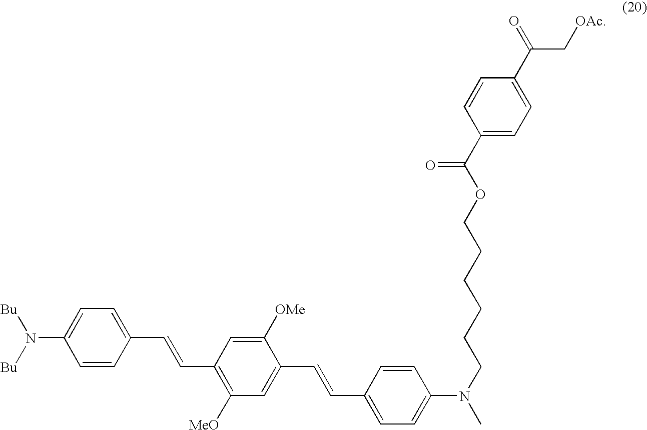

A reaction scheme for the synthesis of Compound (20) is shown below:

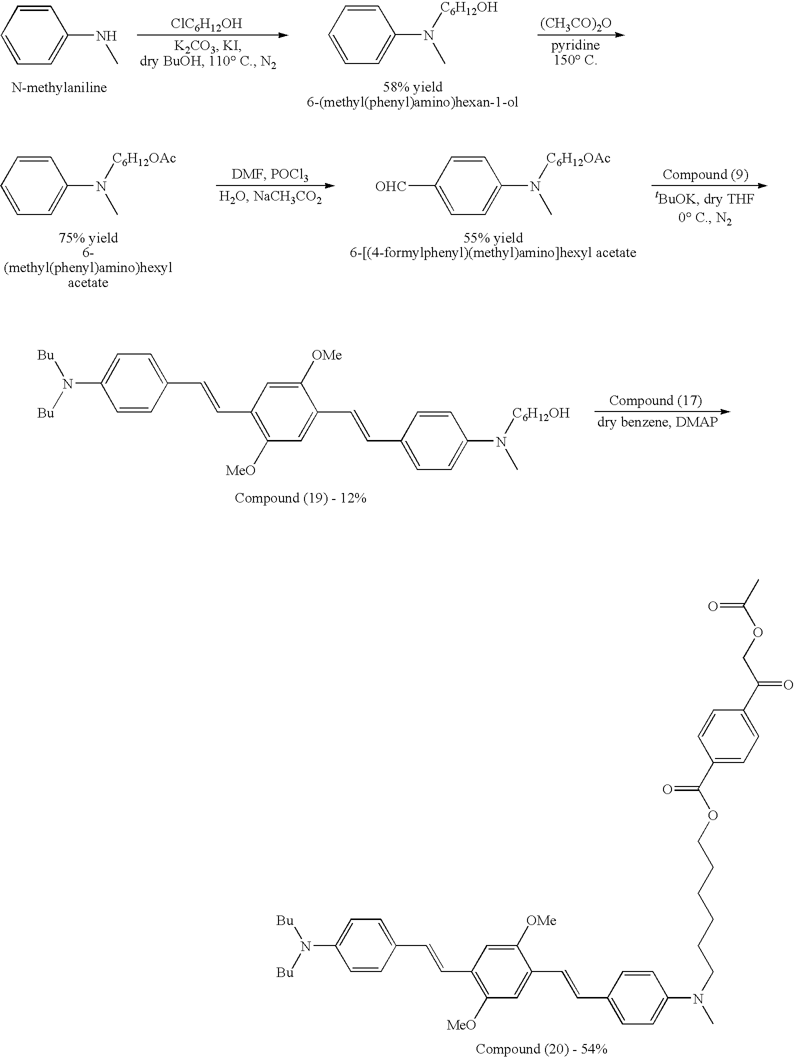

In the first step, a mixture of 18.38 g of N-methylaniline (0.17 mol), 24.62 g of 6-chloro-1-hexanol (0.18 mol), 23.7 g of potassium carbonate (0.17 mol), and 0.21 g of potassium iodide (1.3 mmoL) in 60 mL of dry butanol (60 mL) was heated to 110° C. under nitrogen. After 15 h, the mixture was cooled to room temperature, filtered, and the butanol removed by evaporation. The residue was dissolved in 150 mL of diethyl ether and then extracted with water (3×150 mL). The combined organic layers were dried over magnesium sulfate and the solvent was subsequently removed by evaporation. The residue was fractionally distilled under vacuum resulting in 20.69 g of 6-(methyl(phenyl)amino)hexan-1-ol. The yield was 58% yield. ¹H NMR (500 MHz, chloroform-d) δ 7.21 (t, J=8.5 Hz, 2H), 6.74-6.64 (m, 3H), 3.61 (t, J=6.5 Hz, 2H), 3.29 (t, J=7.5 Hz, 2H), 2.91 (s, 3H), 1.59-1.54 (m, 4H), 1.40-1.34 (m, 5H).

Next, a mixture of 18.49 g of 6-(methyl(phenyl)amino)hexan-1-ol (89.19 mmol, prepared above), 10.41 g of acetic anhydride (99.32 mmol), and 7.85 g of pyridine (99.2 mmol) was heated to 150° C. for 2 h. After the solution was cooled to room temperature, 250 mL of water were added, and the mixture was extracted with ethyl acetate (3×150 mL). The combined organic layers were dried over magnesium sulfate, and the solvent was subsequently removed by evaporation. The residue was fractionally distilled under vacuum resulting in 16.62 g of 6-(methyl(phenyl)amino)hexyl acetate. The yield was 75%. ¹H NMR (500 MHz, chloroform-d) δ 7.21 (t, J=7.5 Hz, 2H), 6.74-6.62 (m, 3H), 4.04 (t, J=6.5 Hz, 2H), 3.29 (t, J=7.5 Hz, 2H), 2.91 (s, 3H), 2.03 (s, 3H), 1.63-1.57 (m, 4H), 1.40-1.34 (m, 4H).

In the next step, 2 mL of phosphorus oxychloride (22 mmol) were added dropwise at 5° C. to 8 mL of N,N-dimethylforamide (DMF), and the reaction mixture was stirred for 2 h. 5.56 g of 6-(methyl(phenyl)amino)hexyl acetate (22.3 mmol, prepared above) were then added slowly, followed by heating the reaction mixture to 90° C. for 3 h. After cooling to room temperature, the solution was poured into ice water, and neutralized to a pH of about 5 with sodium acetate. The mixture was extracted with dichloromethane (3×200 mL). The combined organic layers were dried over magnesium sulfate, and the solvent was subsequently removed by evaporation. Isolation by column chromatography (hexane:ethyl acetate at 4:1, then at 3:1) resulted in 3.38 g of 6-[(4-formylphenyl)(methyl)amino]hexyl acetate. The yield was 55%. ¹H NMR (500 MHz, chloroform-d) δ 9.70 (s, 1H), 7.69 (d, J=8.5 Hz, 2H), 6.66 (d, J=8.5 Hz, 2H), 4.03 (t, J=6.5 Hz, 2H), 3.38 (t, J=7.5 Hz, 2H), 3.02 (s, 3H), 2.02 (s, 3H), 1.65-1.56 (m, 4H), 1.42-1.30 (m, 4H). ¹³C NMR (125 MHz, CDCl₃) δ 190.1, 171.1, 153.2, 132.1, 125.1, 111.0, 64.3, 52.5, 38.6, 28.51, 26.8, 26.6, 25.8, 20.9.

Compound (19), 6-{[4-(2-{4-[2-(4-dibutylamino-phenyl)-vinyl]-2,5-dimethoxy-phenyl}-vinyl)-phenyl]methyl-amino}-hexan-1-ol or 6-((4-(4-(4-(dibutylamino)styryl)-2,5-dimethoxystyryl)phenyl)(methyl)amino)hexan-1-ol, was produced in the next step. To a mixture of 5.09 g of Compound (9) (11.2 mmol, prepared in Example 4) and 3.37 g of 6-[(4-formylphenyl)(methyl)amino]hexyl acetate (12.2 mmol, prepared above) in 60 mL of dry THF, 23 mL of potassium tert-butoxide (23 mmol, 1.0 M in 2-methyl-2-propanol) were added at 0° C. After stirring at 0° C. for 5 h, the solvent was removed by evaporation, the reaction was quenched by adding water, and the resulting mixture was extracted with diethyl ether and dichloromethane until the color of the aqueous layer was only slight yellow or colorless. The combined organic layers were washed with water and dried over magnesium sulfate. Purification by column chromatography (hexane:ethyl acetate at 4:1, and then at 2:1) resulted in 0.76 g of Compound (19). The yield was 12%. ¹H NMR (500 MHz, acetone-d₆) δ 7.38 (d, J=9.0 Hz, 2H), 7.37 (d, J=9.0 Hz, 2H), 7.23 (d, J=16.5 Hz, 1H), 7.24 (s, 1H), 7.23 (s, 1H), 7.22 (d, J=16.5 Hz, 1H), 7.14 (d, J=16.5 Hz, 1H), 7.13 (d, J=16.5 Hz, 1H), 6.70 (d, J=9.0 Hz, 2H), 6.67 (d, J=9.0 Hz, 2H), 3.90 (s, 6H), 3.53 (q, J=6.5 Hz, 2H), 3.39-3.32 (m, 6H), 2.96 (s, 3H), 1.61-1.55 (m, 6H), 1.53-1.50 (m, 2H), 1.42-1.34 (m, 8H), 0.95 (t, J=7.5 Hz, 6H). ¹³C NMR (125 MHz, acetone-d₆) δ 152.1, 152.0, 149.8, 148.7, 129.5, 129.4, 128.5, 128.4, 127.3, 127.1, 126.6, 126.1, 118.9, 118.6, 112.8, 112.6, 109.2, 109.1, 62.3, 56.5, 52.9, 51.2, 38.4, 33.7, 30.2, 27.6, 27.4, 26.6, 20.9, 14.3. HRMS (FAB+) m/z: Calcd. for C₃₉H₅₅N₂O₃ (MH⁺) 599.4213, Found 599.4212. Anal. Calcd. for C₃₉H₅₄N₂O₃: C, 78.22; H, 9.09; N, 4.68. Found: C, 77.56; H, 9.07; N, 4.73.

In the last step, Compound (20) was produced. A mixture of 0.14 g of Compound (19) (0.20 mmol, prepared above), 0.20 mg of Compound (17) (0.83 mmol, prepared in Example 8), and 80 mg of DMAP (0.65 mmol) in 25 mL of dry benzene was heated to reflux for 2 h. After cooling to room temperature, the reaction mixture was quenched by adding 50 mL of a saturated sodium chloride solution, and then extracted with ethyl acetate (3×50 mL). The combined organic layers were dried over magnesium sulfate. Isolation by column chromatography (hexane:ethyl acetate at 2:1) resulted in 0.09 mg of Compound (20). The yield was 54%. ¹H, =9.0 Hz, 2H), 7.36 (d, J=9.0 Hz, 2H), 7.25 (d, J=16.5 Hz, 1H), 7.24 (s, 2H), 7.23 (d, J=16.5 Hz, 1H), 7.14 (d, J=16.5 Hz, 1H), 7.13 (d, J=16.5 Hz, 1H), 6.70 (d, J=9.0 Hz, 2H), 6.67 (d, J=9.0 Hz, 2H), 5.44 (s, 2H), 4.34 (t, J=6.5 Hz, 2H), 3.89 (s, 6H), 3.39 (t, J=6.5 Hz, 2H), 3.34 (t, J=7.5 Hz, 2H), 2.96 (s, 3H), 2.12 (s, 3H), 1.80 (quintet, J=7.5 Hz, 2H), 1.63-1.51 (m, 8H), 1.47-1.41 (m, 2H), 1.37 (sextet, J=7.5 Hz, 4H), 0.95 (t, J=7.5 Hz, 6H). ¹³C NMR (125 MHz, acetone-d₆) δ 192.9, 170.4, 165.9, 152.0, 152.0, 149.8, 148.7, 138.5, 135.5, 130.5, 129.5, 129.4, 128.7, 128.4, 128.3, 127.3, 127.1, 126.6, 126.1, 118.9, 118.6, 112.9, 112.6, 109.2, 109.2, 67.1, 66.0, 56.5, 52.9, 51.2, 38.5, 3.25, 27.4, 27.3, 26.6, 20.9, 20.3, 14.3. HRMS (FAB+) m/z: Calcd. for C₅₀H₆₃N₂O₇ (MH⁺) 803.4635, Found 803.4613.

### Example 10

### Synthesis of a Compound Having at Least One Chromophore Moiety Bonded to at Least One Photocleavable which is Bonded to at Least One Protected Functional Group: 4-(2-acetoxyacetyl)-benzoic acid 3-{2,5-bis-[2-(4-dibutylamino-phenyl)-vinyl]-4-methoxy-phenoxy}-propyl ester

The structure for 4-(2-acetoxyacetyl)-benzoic acid 3-{2,5-bis-[2-(4-dibutylamino-phenyl)-vinyl]-4-methoxy-phenoxy}propyl ester, also referred to as 3-(2,5-bis(4-(dibutylamino)styryl)-4-methoxyphenoxy)propyl 4-(2-acetoxyacetyl)benzoate, is shown below as Compound (23):

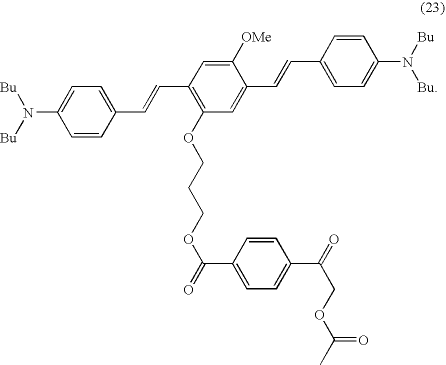

A reaction scheme for the synthesis of Compound (23) is shown below:

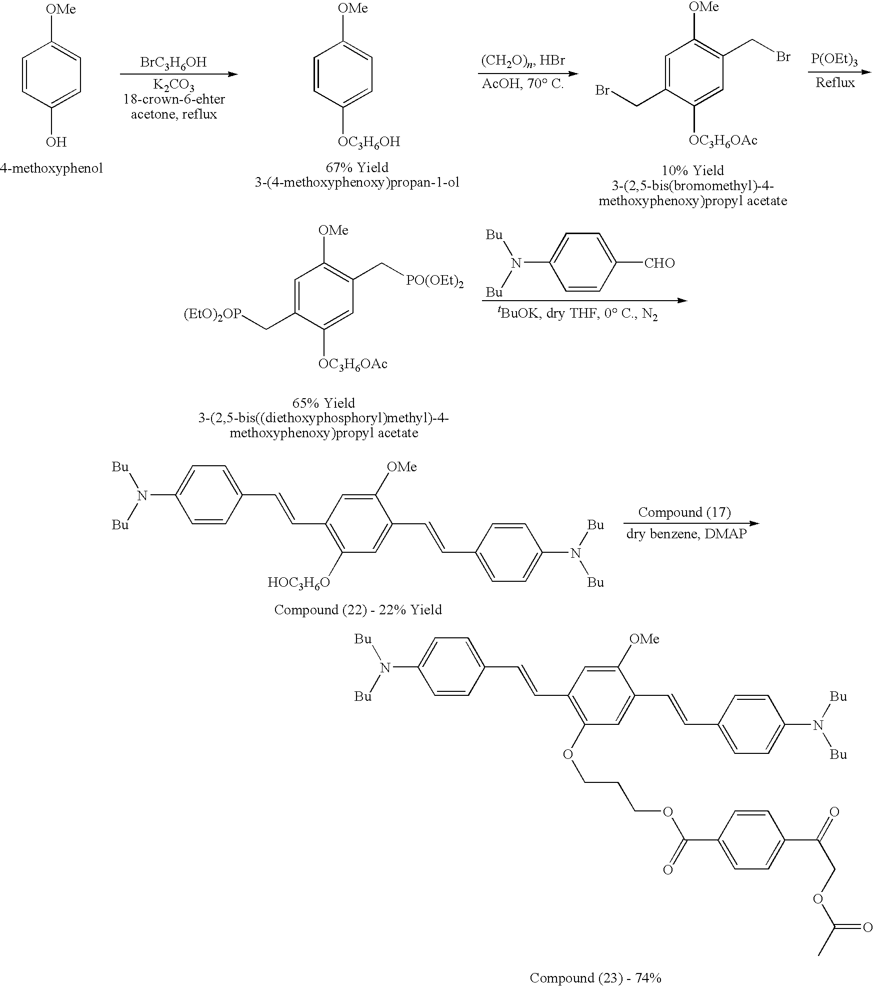

In the first step, a mixture of 15.17 g of 4-methoxylphenol (0.12 mol), 20.06 g of potassium carbonate (0.15 mol), 14.5 mL of 3-bromo-1-propanol (0.16 mol), and 2.77 g of 18-crown-6-ether (0.01 mol) in 100 mL of acetone was heated to reflux for 18 h. After cooling to room temperature, the reaction mixture was quenched by adding 80 mL of water, and then extracted with diethyl ether (3×150 mL). The combined organic layers were dried over magnesium sulfate. Isolation by column chromatography (hexane:ethyl acetate at 2:1) resulted in 14.76 g of 3-(4-methoxyphenoxy)propan-1-ol. The yield was 67% yield. ¹H NMR (500 MHz, chloroform-d) δ 6.81 (d, J=2.0 Hz, 4H), 4.06 (t, J=6.0 Hz, 2H), 3.84 (t, J=6.0 Hz, 2H), 3.75 (s, 3H), 2.00 (quintet, J=6.0 Hz, 2H). ¹³C NMR (125 MHz, chloroform-d) δ 153.9, 152.9, 115.5, 114.7, 66.7, 60.7, 55.7, 32.1.

Next, to a mixture of 5.33 g of 3-(4-methoxyphenoxy)-propan-1-ol (23.8 mmol, prepared above) and 1.57 g of paraformaldehyde (49.7 mmol) in 30 mL of acetic acid, 24 mL of hydrogen bromide (33% wt in acetic acid, 18.8 mmol) were added. The reaction mixture was heated to 70° C. for 4 h. After cooling to room temperature, the reaction mixture was poured into 1500 mL of water, and subsequently extracted with dichloromethane (3×500 mL). Isolation by column chromatography (hexane:ethyl acetate at 7:1) resulted in 0.94 g of 3-(2,5-bis(bromomethyl)-4-methoxyphenoxy)propyl acetate). The yield was 10%. ¹H NMR (500 MHz, chloroform-d) δ 6.85 (s, 1H), 6.84 (s, 1H), 4.50 (s, 2H), 4.49 (s, 2H), 4.30 (t, J=6.0 Hz, 2H), 4.07 (t, J=6.0 Hz, 2H), 3.85 (s, 3H), 2.14 (quintet, J=6.0 Hz, 2H), 2.06 (s, 3H).

In the next step, a mixture of 3.13 g of 3-(2,5-bis(bromomethyl)-4-methoxyphenoxy)propyl acetate) (7.63 mmol, prepared above) and 30 mL of triethyl phosphite was refluxed for 18 h. The triethyl phosphite was removed by distillation. Purification by column chromatography (dichloromethane:methanol at 30:1) resulted in 2.59 g of 3-(2,5-bis((diethoxyphosphoryl)methyl)-4-methoxyphenoxy)propyl acetate. The yield was 65%. ¹H NMR (500 MHz chloroform-d) δ 6.89 (s, 1H), 6.87 (s, 1H), 4.23 (t, J=6.0 Hz, 2H), 3.99-3.97 (m, 8H), 3.76 (s, 3H), 3.19 (s, 2H), 3.15 (s, 2H), 2.07 (quintet, J=7.5 Hz, 2H), 2.02 (s, 2H), 1.20 (t, J=7.5 Hz, 12H).

Compound (22), 3-{2,5-Bis-[2-(4-dibutylamino-phenyl)-vinyl]-4-methoxy-phenoxy}-propan-1-ol or 3-(2,5-bis(4-(dibutylamino)styryl)-4-methoxyphenoxy)propan-1-ol, was produced in the next step. To a mixture of 2.59 g of 3-(2,5-bis((diethoxyphosphoryl)methyl)-4-methoxyphenoxy)propyl acetate (4.94 mmol, prepared above) and 2.5 mL of (dibutylamino)benzaldehyde (10 mmol) in 20 mL of dry THF, 13 mL of potassium tert-butoxide (13 mmol, 1.0 M in 2-methyl-2-propanol) were added at 0° C. After stirring at 0° C. for 4 h and then at room temperature for 3 h, the reaction was quenched by adding water, and subsequently extracted with ether and dichloromethane until the color of the aqueous layer was only slight yellow or colorless.

The combined organic layers were washed with water and dried over magnesium sulfate. Column chromatography (chloromethane:methanol at 20:1) resulted in 0.7 g of Compound (22). The yield was 22%. ¹H NMR (500 MHz, acetone-d₅) δ 7.35 (d, J=9.0 Hz, 4H), 7.25 (s, 1H), 7.24 (d, J=16.5 Hz, 1H), 7.22 (d, J=16.5 Hz, 1H), 7.21 (s, 1H), 7.14 (d, J=16.5 Hz, 1H), 7.11 (d, J=16.5 Hz, 1H), 6.66 (d, J=9.0 Hz, 42H), 4.17 (t, J=6.5 Hz, 2H), 3.88 (s, 6H), 3.82 (m, 2H), 3.32 (t, J=7.5 Hz, 8H), 2.04 (quintet, J=6.0 Hz, 2H) 1.57 (quintet, J=7.5 Hz, 8H), 1.35 (sextet, J=7.5 Hz, 8H), 0.94 (t, J=7.5 Hz, 12H). ¹³C NMR (125 MHz, acetone-d₆) δ 152.1, 151.5, 148.7, 129.4, 128.5, 128.4, 127.5, 127.2, 126.2, 126.1, 118.8, 118.6, 112.6, 110, 109.1, 66.9, 59.4, 56.5, 51.3, 51.2, 33.7, 30.3, 20.9, 14.3. HRMS (FAB+) m/z: Calcd for C₄₂H₆₁N₂O₃ (MH⁺) 641.4682, Found 641.4690. Anal. Calcd. for C₄₂H₆₀N₂O₃: C, 78.48; H, 9.44; N, 4.37. Found: C, 78.48; H, 9.55; N, 4.39.

In the last step, Compound (23) was produced. A mixture of 0.52 g of Compound (22) (0.81 mmol, prepared above), 0.59 mg of Compound (17) (2.5 mmol, prepared in Example 8), and 0.3 g of DMAP (2.5 mmol) in 25 mL of dry benzene was heated to reflux for 2 h. After cooling to room temperature, the reaction mixture was quenched by adding 80 mL of a saturated sodium chloride solution, followed by extraction with ethyl acetate (3×80 mL). The combined organic layers were dried over magnesium sulfate. Isolation by column chromatography (hexane:ethyl acetate at 8:1 and then at 4:1) resulted in 0.5 mg of Compound (23). The yield was 74%. ¹H NMR (500 MHz, acetone-d₆) δ 8.15 (d, J=8.5 Hz, 2H), 8.00 (d, J=8.5 Hz, 2H), 7.38 (d, J=8.5 Hz, 2H), 7.34 (d, J=8.5 Hz, 2H), 7.30 (d, J=16.5 Hz, 1H), 7.28 (s, 1H), 7.24 (d, J=16.5 Hz, 1H), 7.23 (s, 1H), 7.15 (d, J=16.5 Hz, 1H), 7.08 (d, J=16.5 Hz, 1H), 6.66 (d, J=8.5 Hz, 2H), 6.65 (d, J=8.5 Hz, 2H), 5.34 (s, 1H), 4.64 (t, J=6.0 Hz, 2H), 4.28 (t, J=6.0 Hz, 2H), 3.89 (s, 3H), 3.34-3.31 (m, 8H), 2.36 (quintet, J=6.0 Hz, 2H), 2.12 (s, 3H), 1.59-1.54 (m, 8H), 1.37 (sextet, J=7.5 Hz, 8H), 0.95 (sextet, J=7.5 Hz, 12H). ¹³C NMR (125 MHz, acetone-d₆) δ 192.8, 170.3, 165.9, 152.3, 151.2, 148.7, 138.4, 135.4, 130.6, 129.7, 129.5, 128.6, 128.5, 128.4, 127.7, 127.2, 126.0, 118.5, 112.6, 112.6, 111.1, 109.0, 67.0, 66.9, 63.4, 56.5, 51.2, 30.3, 20.9, 20.4, 14.3. HRMS (FAB+) m/z: Calcd. for C₅₃H₆₉N₂O₇ (MH⁺) 845.5105, Found 845.5111.

### Example 11

### Intermolecular Photodeprotection Experiment Using a Chromophore Compound and a Second Compound Having a Photocleavable Group Bonded to a Protected Functional Group

A Rayonet photochemical reactor equipped with fourteen lamps (419 nm) was used as the single photon radiation light source (one-photon excitation) for the chromophore to initiate electron transfer to the photocleavable group to cleave and deprotect the protected functional group. ¹H NMR spectra were taken to follow the progress of the reaction. A solution of the chromophore (1.5×10⁻² M), the second compound (1.5×10⁻² M), and hexamethyldisiloxane (3 μL) as an internal standard in benzene-d₆ (2 mL) was prepared. 1 mL of the solution was transferred to a sealed NMR tube through a mini-filter and purged with nitrogen gas for about 15 min. ¹H NMR spectrum was taken, and the areas of corresponding peaks (see reactions schemes below and associated figures) relative to hexamethyldisiloxane were determined and recorded. As irradiation time progressed, the relative peak areas of the second compound (dis=disappeared) and the photoproduct (app=appeared) were recorded at different irradiation time. The integration was plotted versus the irradiation time for successful photodeprotection reactions.

In this example, Compound (11), also referred to as dye 41 or 4,4′-(1E,1′E)-2,2′-(2,5-dimethoxy-1,4-phenylene)bis(ethene-2,1-diyl)bis(N,N-dibutylaniline) was the chromophore compound. Compound (5), methyl 4-(2-acetoxyacetyl)benzoate, was the second compound. A reaction scheme for Example 11 is shown below:

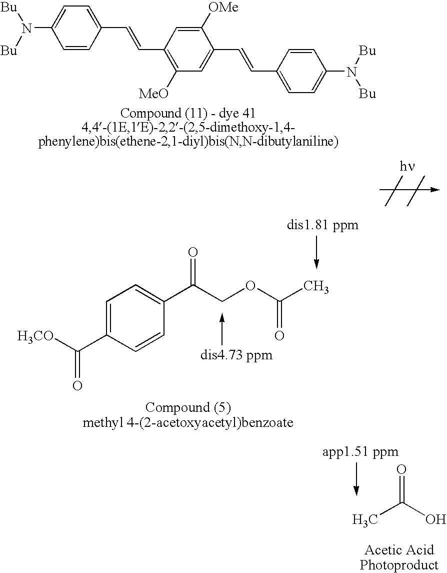

In Example 11, the photodeprotection scheme was unsuccessful in cleaving and deprotecting the acetic acid functional group.

### Example 12

### Intermolecular Photodeprotection Experiment Using a Chromophore Compound and a Second Compound Having a Photocleavable Group Bonded to a Protected Functional Group

Substantially the same procedure discussed above relative to Example 11 was employed in Example 12. Compound (11) was employed in Example 12; however, the second compound was Compound (1), acetic acid 2-oxo-2-phenyl-ethyl ester or 2-oxo-2-phenylethyl acetate. A reaction scheme for Example 12 is shown below:

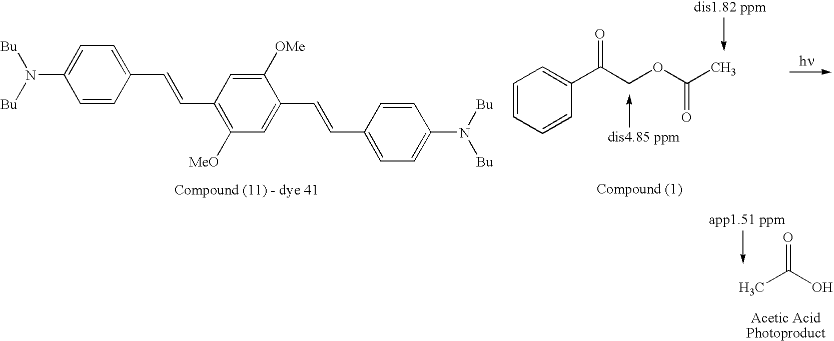

FIG. 21 demonstrates that the reaction scheme of Example 12 was successful in cleaving and deprotecting a protected functional group—in this case, acetic acid. As irradiation time progressed, moieties at 1.82 ppm and 4.85 ppm decreased, while the acetic acid moiety at 1.51 ppm increased. Thus, due to absorption of radiation, electrons were transferred from Compound (11) to Compound (1) causing the protected functional group to be cleaved from Compound (1).

### Example 13

### Intermolecular Photodeprotection Experiment Using a Chromophore Compound and a Second Compound Having a Photocleavable Group Bonded to a Protected Functional Group

Substantially the same procedure discussed above relative to Example 11 was employed in Example 13. Compound (11) was employed in Example 13; however, the second compound was Compound (4), 2-(4-methoxyphenyl)-2-oxoethyl acetate. A reaction scheme for Example 13 is shown below:

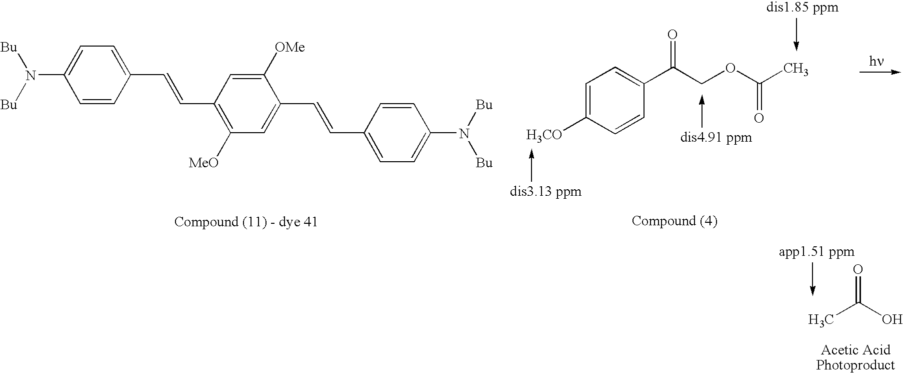

FIG. 22 demonstrates that the reaction scheme of Example 13 was successful in cleaving and deprotecting a protected functional group—in this case, acetic acid. As irradiation time progressed, moieties at 1.85 ppm, 3.13 ppm, and 4.91 ppm decreased, while the acetic acid moiety at 1.51 ppm increased. Thus, due to absorption of radiation, electrons were transferred from Compound (11) to Compound (4) causing the protected functional group to be cleaved from Compound (4).

### Example 14

### Intramolecular Photodeprotection Experiment Using a Compound Comprising a Chromophore Moiety Bonded to a Photocleavable Group which is Bonded to a Protected Functional Group

Substantially the same procedure discussed above relative to Example 11 was employed in Example 14. Instead of a chromophore compound and a second compound having a photocleavable group bonded to a protected functional group, however, only Compound (21) was employed. Compound (21) is a compound comprising a chromophore moiety bonded to a photocleavable group which is bonded to a protected functional group. See Reaction Scheme D above for a non-limiting example of the synthesis of Compound (21). A reaction scheme for Example 14 is shown below:

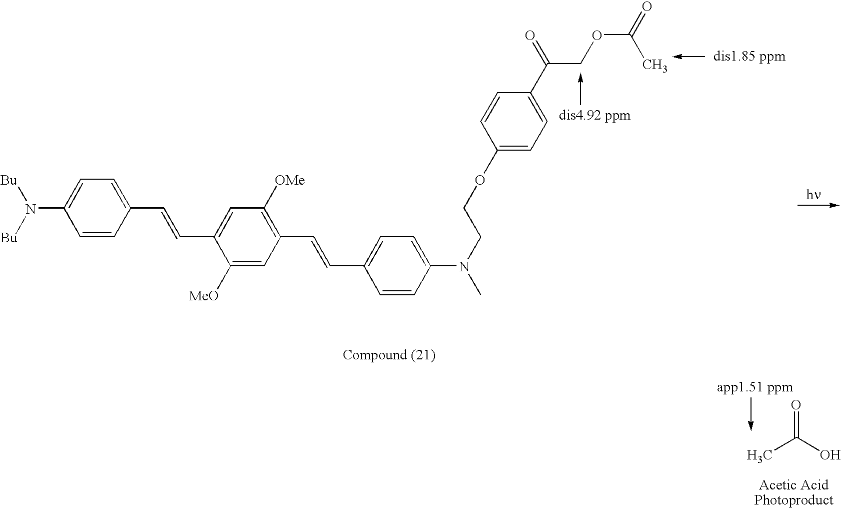

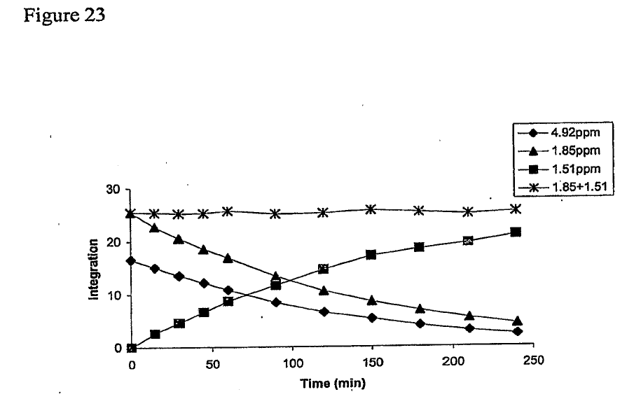

FIG. 23 demonstrates that the reaction scheme of Example 14 was successful in cleaving and deprotecting a protected functional group—in this case, acetic acid. As irradiation time progressed, moieties at 1.85 ppm and 4.91 ppm decreased, while the acetic acid moiety at 1.51 ppm increased. Thus, due to absorption of radiation, electrons were transferred from the chromophore moiety of Compound (21) to the photocleavable group within the same compound, causing the protected functional group to be cleaved from Compound (21).

### Constructive Example 15

### Constructive Intermolecular Photodeprotection Experiment Using Two Photon Excitation, a Chromophore Compound, and a Second Compound Having a Photocleavable Group Bonded to a Protected Functional Group

Constructive Example 15 substantially employs the procedure described in Example 11. A photochemical reactor can be equipped with a light source at 730 nm and used as a two-photon radiation light source (two-photon excitation) for the chromophore to initiate electron transfer to the photocleavable group to cleave and deprotect the protected functional group. ¹H NMR spectra is taken to follow the progress of the reaction. A solution of the chromophore (1.5×10⁻² M), the second compound (1.5×10⁻² M), and hexamethyldisiloxane (3 μL) as an internal standard in benzene-d₆ (2 mL) is prepared. 1 mL of the solution is transferred to a sealed NMR tube through a mini-filter and purged with nitrogen gas for about 15 min. ¹H NMR spectrum is taken, and the areas of corresponding peaks (see reactions schemes and associated figures in Examples 12-14) relative to hexamethyldisiloxane is determined and recorded. As irradiation time progresses, the relative peak areas of the second compound (dis=disappeared) and the photoproduct (app=appeared) are recorded at different irradiation time. The integration is plotted versus the irradiation time for successful photodeprotection reactions.

Constructive Example 15 employs the same material described in Example 12. Compound (11) is used as the chromophore compound. Compound (1) is the second compound. A reaction scheme for Constructive Example 15 is shown below:

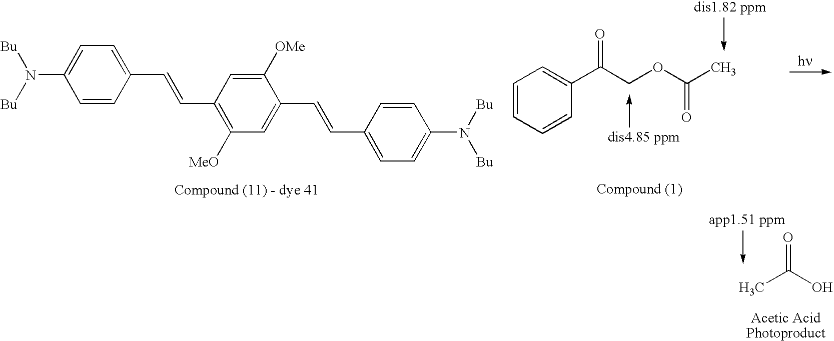

Compound (11), also referred to as dye 41, has a two-photon absorption cross-section of about 900×10⁻⁵⁰ cm⁴s/photon at 730 nm wavelength, as disclosed in Rumi et al., J. Am. Chem. Soc., 2000, 122, 9500-9510. Therefore, excitation of this compound with two-photon radiation at 730 nm is expected to result in the transfer of electrons from Compound (11) to Compound (1) causing the protected functional group to be cleaved from Compound (1). Constructive Example 15 shows that excitation followed by electron transfer and cleavage using two-photon excitation is analogous to that achievable by one-photon excitation.

### Constructive Example 16

### Constructive Intramolecular Photodeprotection Experiment Using Two-Photon Excitation and a Compound Comprising a Chromophore Moiety Bonded to a Photocleavable Group which is Bonded to a Protected Functional Group

Constructive Example 16 substantially employs the procedure described in Constructive 15. Instead of a chromophore compound and a second compound having a photocleavable group bonded to a protected functional group, however, only Compound (21) is employed. Compound (21) is a compound comprising a chromophore moiety bonded to a photocleavable group which is bonded to a protected functional group. A reaction scheme for Constructive Example 16 is shown below:

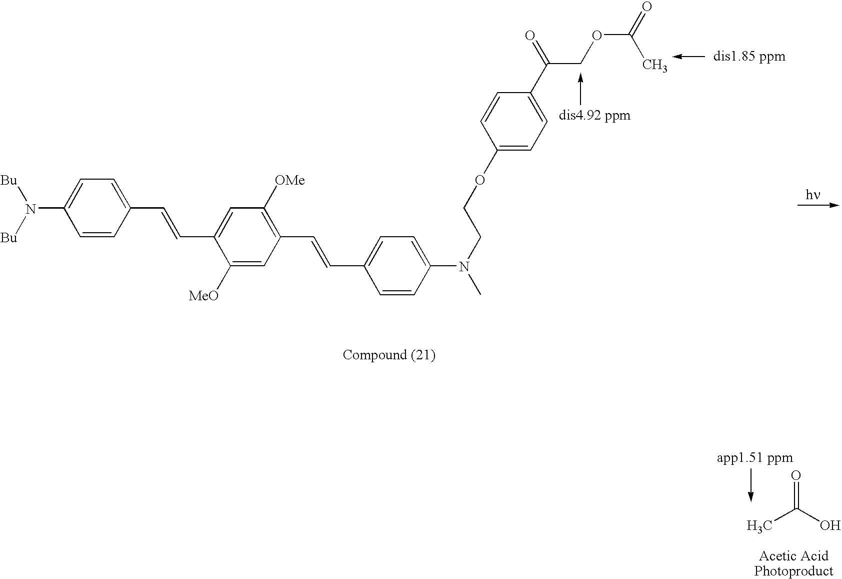

Since Compound (21) has a chromophore moiety similar to that of Compound (11), it is expected that Compound (21) would have a two-photon absorption cross-section of about 900×10⁻⁵⁰ cm⁴s/photon at 730 nm wavelength. Therefore, excitation of this compound with two-photon radiation at 730 nm is expected to result in the transfer of electrons from the chromophore moiety of Compound (21) to the photocleavable group within the same compound, causing the protected functional group to be cleaved from Compound (21). Constructive Example 16 shows that excitation followed by electron transfer and cleavage using two-photon excitation is analogous to that achievable by one-photon excitation, in intramolecular or intermolecular photodeprotection.

1. A composition comprising:
(a) at least one chromophore compound selected from: 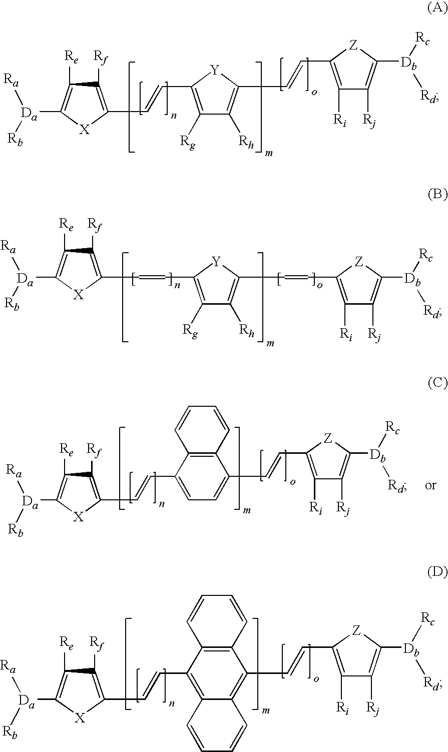
wherein:
Dₐ is selected from N, O, S, or P;
D_{b} is selected from N, O, S, or P;
m, n, and o independently are integers from 0 to 10, inclusive;
X, Y, and Z independently are selected from CRₖ═CRₗ, O, S, or N—Rₘ;
Rₐ, R_{b}, R_{c}, and R_{d} independently are selected from a hydrogen atom, a linear or branched alkyl group with up to 25 carbons, —(CH₂CH₂O)_{α}—(CH₂)_{β}Rₐ₁, —(CH₂CH₂O)_{α}—(CH₂)_{β}NRₐ₂Rₐ₃, —(CH₂CH₂O)_{α}-(CH₂)_{β}CONRₐ₂Rₐ₃, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, a group of aromatic rings having up to 20 carbons in the aromatic ring framework, fused aromatic rings, vinyl, allyl, 4-styryl, acroyl, methacroyl, acrylonitrile, isocyanate, isothiocyanate, epoxides, strained ring olefins, (—CH₂)_{γ}SiCl₃, (—CH₂)_{γ}Si(OCH₂CH₃)₃, or (—CH₂)_{γ}Si(OCH₃)₃; wherein one of Rₐ and R_{b} is not present when Dₐ is O or S and wherein one of R_{c} and R_{d} is not present when D_{b} is O or S;
Rₑ, R_{f}, R_{g}, Rₕ, Rᵢ, Rⱼ, Rₖ, Rₗ, and Rₘ independently are selected from a hydrogen atom, a linear or branched alkyl group with up to 25 carbons, —(CH₂CH₂O)_{α}—(CH₂)_{β}OR_{b1}, —(CH₂CH₂O)_{α}—(CH₂)_{β}NR_{b2}R_{b3}, —(CH₂CH₂O)_{α}—(CH₂)_{β}CONR_{b2}R_{b3}, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, a group of aromatic rings having up to 20 carbons in the aromatic framework, fused aromatic rings, CHO, CN, NO₂, Br, Cl, I, phenyl, an acceptor group containing more than two carbon atoms, a functional group obtained by reaction with an amino acid, NRₑ₁Rₑ₂, or ORₑ₃;
Rₐ₁, Rₐ₂, and Rₐ₃ independently are selected from a hydrogen atom, a linear or branched alkyl group with up to 25 carbons, or a functional group obtained by reaction with: an amino acid, a polypeptide, adenine, guanine, tyrosine, cytosine, uracil, biotin, ferrocene, ruthenocene, cyanuric chloride, or methacryloyl chloride;
R_{b1}, R_{b2}, and R_{b3} independently are selected from a functional group obtained by reaction with: an amino acid, a polypeptide, adenine, guanine, tyrosine, cytosine, uracil, biotin, ferrocene, ruthenocene, cyanuric chloride, or methacryloyl chloride;
Rₑ₁, Rₑ₂, and Rₑ₃, are independently selected from a hydrogen atom, a linear or branched alkyl group with up to 25 carbons, —(CH₂CH₂O)_{α}—(CH₂)_{β}OR_{g1}, —(CH₂CH₂O)_{α}—(CH₂)_{β}NR_{g2}R_{g3}, —(CH₂CH₂O)_{α}—(CH₂)_{β}CONR_{g2}R_{g3}, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, aryl groups, fused aromatic rings, vinyl, allyl, 4-styryl, acroyl, methacroyl, acrylonitrile, isocyanate, isothiocyanate, epoxides, strained ring olefins, (—CH₂)_{γ}SiCl₃, (—CH₂)_{γ}Si(OCH₂CH₃)₃, or (—CH₂)_{γ}Si(OCH₃)₃;
R_{g1}, R_{g2}, and R_{g3} independently are selected from a hydrogen atom, a linear or branched alkyl group with up to 25 carbons, or a functional group obtained by reaction with: an amino acid, a polypeptide, adenine, guanine, tyrosine, cytosine, uracil, biotin, ferrocene, ruthenocene, cyanuric chloride, or methacryloyl chloride;
α is an integer from 0 to 10, inclusive;
β and γ independently are integers in a range from 1 to 25, inclusive; and
(b) at least one second compound having at least one photocleavable group bonded to at least one protected group,
wherein the at least one photocleavable group has the formula: 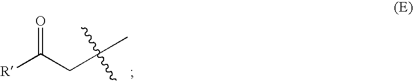
wherein R′ is a substituted or unsubstituted aryl or heteroaryl moiety, wherein any substituents on R′ are selected from alkyl, alkenyl, alkoxy, or hydroxy groups; and
wherein the at least one protected group is selected from selected from: 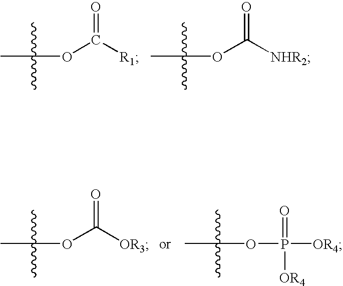
wherein:
R₁, R₂, R₃, and R₄ independently are selected from a substituted or unsubstituted alkyl, alkenyl, alkynyl, aryl, aralkyl, aralkenyl, aralkynyl, fused aryl, carbocyclic, carboxylalkyl-substituted alkyl, heterocycloalkyl, heterocycloalkyl-substituted alkyl, heteroaryl, heteroarylalkyl, heteroarylalkenyl, or heteroaralkynyl groups, wherein any substituents on R₁, R₂, R₃, and R₄ independently are selected from alkyl, alkenyl, alkynyl, alkoxy, acyl (alkanoyl), acyloxy, cyano, alkylcarboxy, chloro, bromo, aryl, cycloalkyl, aralkyl, aralkenyl, aralkynyl, hydroxy, nitro, amino, carboxy, amino acid, peptide, adenine, guanine, tyrosine, cytosine, uracil, biotin, ferrocene, ruthenocene, cyanuric chloride, methacryloyl chloride, glucose, mannose, galactose, gulose, allose, altrose, idose, talose, fructose, arabinose, xylose, sucrose, cellobiose, maltose, lactose, trehalose, gentiobiose, melibiose, raffinose, gentianose, adenosine, deoxyadenosine, guanosine, deoxyguanosine, cytidine, deoxycytidine, uridine, or deoxythymidine; and
wherein the rate constant for cleavage, k_{cleav}, of a charge-transfer state of the photocleavable group is greater than 0.1 times the rate constant for back-electron transfer, k_{BET}, of the charge-transfer state of the photocleavable group.

2. A composition comprising:
(a) at least one chromophore compound selected from: 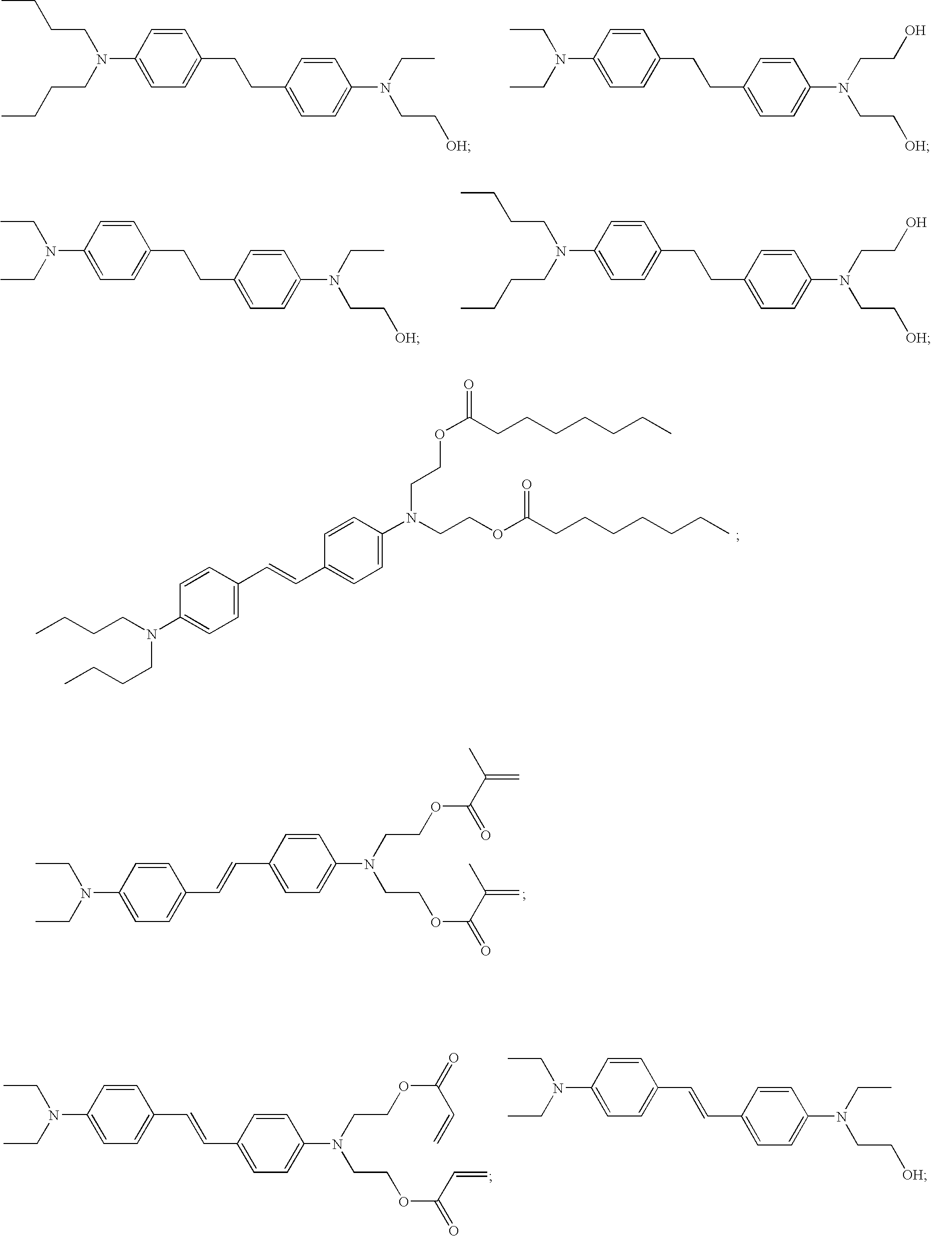 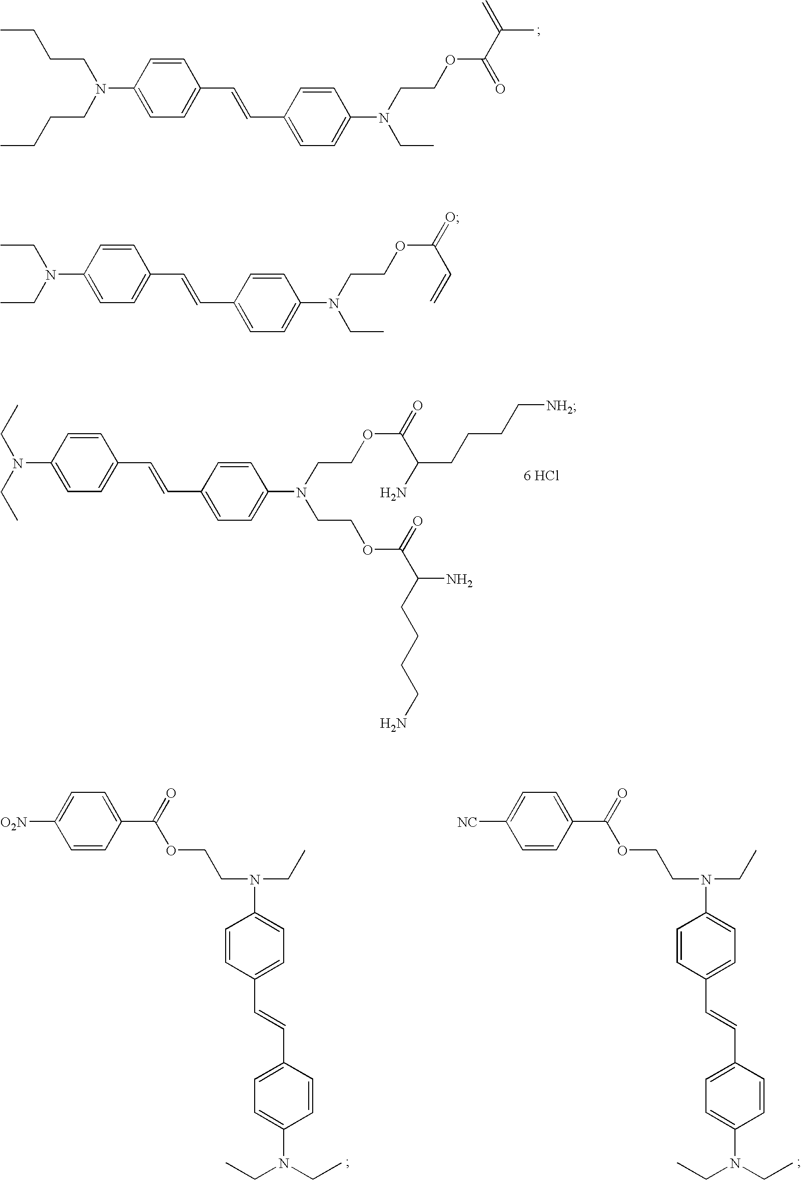 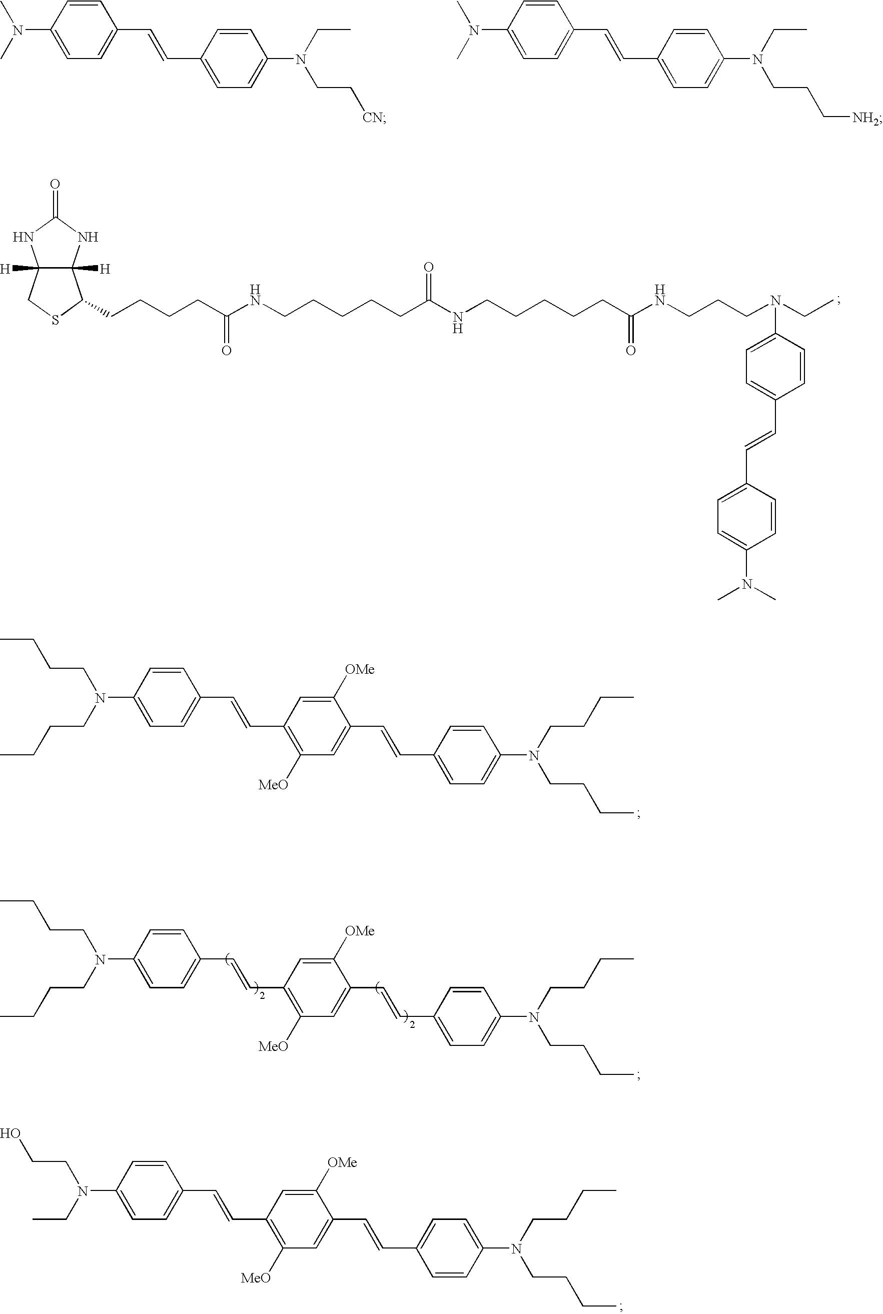 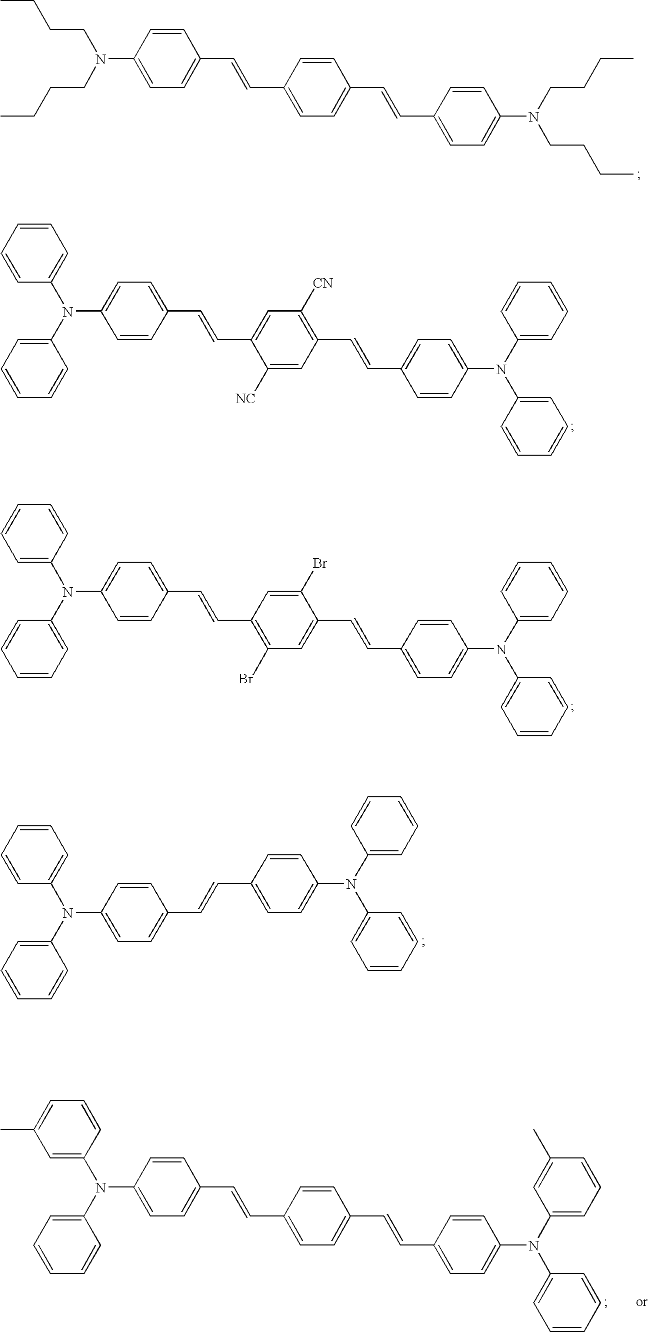 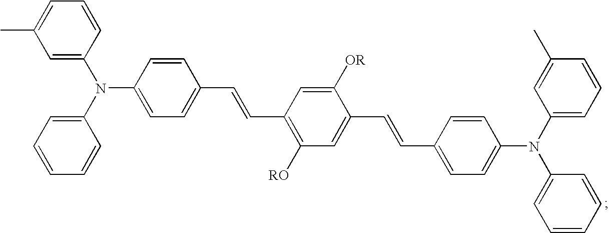
or a combination thereof, wherein R═(CH₂)₁₁CH₃; and
(b) at least one second compound having at least one photocleavable group bonded to at least one protected group,
wherein the at least one photocleavable group has the formula: 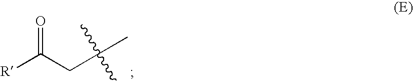
wherein R′ is a substituted or unsubstituted aryl or heteroaryl moiety, wherein any substituents on R′ are selected from alkyl, alkenyl, alkoxy, or hydroxy groups; and
wherein the at least one protected group is selected from selected from: 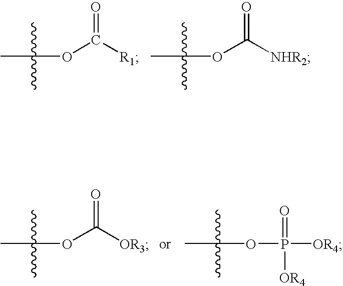
wherein:
R₁, R₂, R₃, and R₄ independently are selected from a substituted or unsubstituted alkyl, alkenyl, alkynyl, aryl, aralkyl, aralkenyl, aralkynyl, fused aryl, carbocyclic, carboxylalkyl-substituted alkyl, heterocycloalkyl, heterocycloalkyl-substituted alkyl, heteroaryl, heteroarylalkyl, heteroarylalkenyl, or heteroaralkynyl groups, wherein any substituents on R₁, R₂, R₃, and R₄ independently are selected from alkyl, alkenyl, alkynyl, alkoxy, acyl (alkanoyl), acyloxy, cyano, alkylcarboxy, chloro, bromo, aryl, cycloalkyl, aralkyl, aralkenyl, aralkynyl, hydroxy, nitro, amino, carboxy, amino acid, peptide, adenine, guanine, tyrosine, cytosine, uracil, biotin, ferrocene, ruthenocene, cyanuric chloride, methacryloyl chloride, glucose, mannose, galactose, gulose, allose, altrose, idose, talose, fructose, arabinose, xylose, sucrose, cellobiose, maltose, lactose, trehalose, gentiobiose, melibiose, raffinose, gentianose, adenosine, deoxyadenosine, guanosine, deoxyguanosine, cytidine, deoxycytidine, uridine, or deoxythymidine; and
wherein the rate constant for cleavage, k_{cleav}, of a charge-transfer state of the photocleavable group is greater than 0.1 times the rate constant for back-electron transfer, k_{BET}, of the charge-transfer state of the photocleavable group.

3. The composition of claim 1, wherein k_{cleav} is greater than 0.5 times k_{BET}.

4. The composition of claim 1, wherein k_{cleav} is greater than k_{BET}.

5. The composition of claim 1, wherein k_{cleav} is greater than 5 times k_{BET}.

6. The composition of claim 1, wherein the chromophore compound has a two-photon absorption cross-section of greater than 50×10⁻⁵⁰ cm⁴s/photon.

7. The composition of claim 1, wherein the chromophore compound has a two-photon absorption cross-section of greater than 100×10⁻⁵⁰ cm⁴s/photon.

8. The composition of claim 1, wherein the chromophore compound has a two-photon absorption cross-section of greater than 500×10⁻⁵⁰ cm⁴s/photon.

9. The composition of claim 1, wherein the Gibbs free energy change associated with electron transfer from the chromophore compound to the second compound (ΔG_{ET}) is less than +28 kJ/mol.

10. The composition of claim 1, wherein the Gibbs free energy change associated with electron transfer from the chromophore compound to the second compound (ΔG_{ET}) is less than +14 kJ/mol.

11. The composition of claim 1, wherein the Gibbs free energy change associated with electron transfer from the chromophore compound to the second compound (ΔG_{ET}) is less than zero.

12. The composition of claim 1, wherein the rate constant for electron transfer, k_{ET}, of an excited state of the chromophore compound is greater than 0.1×(rate constant for radiative decay plus the rate constant for non-radiative decay) (k_{rad}+k_{non-rad}) of the excited state of the chromophore compound.

13. The composition of claim 1, wherein the rate constant for electron transfer, k_{ET}, of an excited state of the chromophore compound is greater than 0.5×(rate constant for radiative decay plus the rate constant for non-radiative decay) (k_{rad}+k_{non-rad}) of the excited state of the chromophore compound.

14. The composition of claim 1, wherein the rate constant for electron transfer, k_{ET}, of an excited state of the chromophore compound is greater than the rate constant for radiative decay plus the rate constant for non-radiative decay (k_{rad}+k_{non-rad}) of the excited state of the chromophore compound.

15. The composition of claim 1, wherein the electrostatic interaction between the at least one chromophore compound and the at least one second compound is greater than 3 kcal/mol when the at least one chromophore compound and the at least one second compound, independently, are present in a solution at a concentration between about 0.001M and about 2M.

16. A method for deprotecting a protected functional group comprising:
(a) viding a composition according to claim 1;
(b) exposing the composition to radiation;
(c) converting the chromophore compound to a multi-photon electronically excited state upon simultaneous absorption of at least two photons of the radiation by the chromophore compound, wherein the sum of the energies of all of the absorbed photons is greater than or equal to the transition energy from a ground state of the chromophore compound to the multi-photon excited state and wherein the energy of each absorbed photon is less than the transition energy between the ground state and the lowest single-photon excited state of the chromophore compound and is less than the transition energy between the multi-photon excited state and the ground state;
thereby inducing an electron transfer from the chromophore compound to the photocleavable group in the second compound to cleave and deprotect the protected functional group in the second compound.

17. A compound comprising at least one chromophore moiety bonded to at least one photocleavable group, the at least one photocleavable group bonded to at least one protected group, wherein:
(a) the at least one chromophore moiety is selected from: 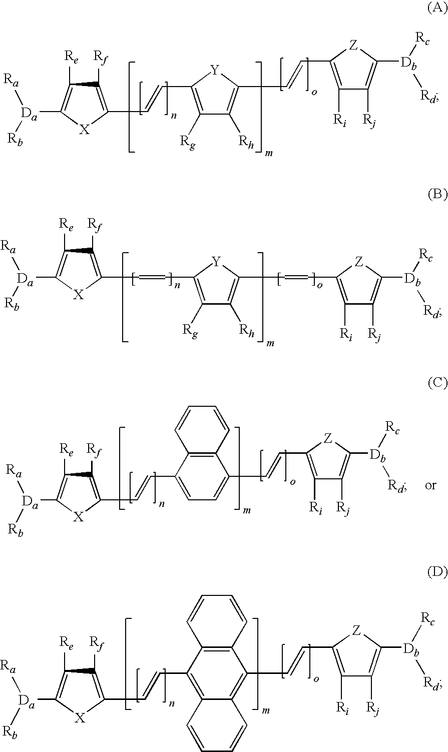
wherein:
Dₐ is selected from N, O, S, or P;
D_{b} is selected from N, O, S, or P;
m, n, and o independently are integers from 0 to 10, inclusive;
X, Y, and Z independently are selected from CRₖ═CRₗ, O, S, or N—Rₘ;
Rₐ, R_{b}, R_{c}, and R_{d} independently are selected from a hydrogen atom, a linear or branched alkyl group with up to 25 carbons, —(CH₂CH₂O)_{α}—(CH₂)_{β}ORₐ₁, —(CH₂CH₂O)_{α}—(CH₂)_{β}NRₐ₂Rₐ₃, —(CH₂CH₂O)_{α}—(CH₂)_{β}CONRₐ₂Rₐ₃, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, a group of aromatic rings having up to 20 carbons in the aromatic ring framework, fused aromatic rings, vinyl, allyl, 4-styryl, acroyl, methacroyl, acrylonitrile, isocyanate, isothiocyanate, epoxides, strained ring olefins, (—CH₂)_{γ}SiCl₃, (—CH₂)_{γ}Si(OCH₂CH₃)₃, or (—CH₂)_{γ}Si(OCH₃)₃; wherein one of Rₐ and R_{b} is not present when Dₐ is O or S and wherein one of R_{c} and R_{d} is not present when D_{b} is O or S;
Rₑ, R_{f}, R_{g}, Rₕ, Rᵢ, Rⱼ, Rₖ, Rₗ, and Rₘ independently are selected from a hydrogen atom, a linear or branched alkyl group with up to 25 carbons, —(CH₂CH₂O)_{α}—(CH₂)_{β}OR_{b1}, —(CH₂CH₂O)_{α}—(CH₂)_{β}NR_{b2}R_{b3}, —(CH₂CH₂O)_{α}—(CH₂)_{β}CONR_{b2}R_{b3}, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}, —(CH₂)_{β}-Phenyl, a group of aromatic rings having up to 20 carbons in the aromatic framework, fused aromatic rings, CN, NO₂, Br, Cl, I, phenyl, an acceptor group containing more than two carbon atoms, a functional group obtained by reaction with an amino acid, NRₑ₁Rₑ₂, or ORₑ₃;
Rₐ₁, Rₐ₂, and Rₐ₃ independently are selected from a hydrogen atom, a linear or branched alkyl group with up to 25 carbons, or a functional group obtained by reaction with: an amino acid, a polypeptide, adenine, guanine, tyrosine, cytosine, uracil, biotin, ferrocene, ruthenocene, cyanuric chloride, or methacryloyl chloride;
R_{b1}, R_{b2}, and R_{b3} independently are selected from a functional group obtained by reaction with: an amino acid, a polypeptide, adenine, guanine, tyrosine, cytosine, uracil, biotin, ferrocene, ruthenocene, cyanuric chloride, or methacryloyl chloride;
Rₑ₁, Rₑ₂e, and Rₑ₃, are independently selected from a hydrogen atom, a linear or branched alkyl group with up to 25 carbons, —(CH₂CH₂O)_{α}—(CH₂)_{β}OR_{g1}, —(CH₂CH₂O)_{α}—(CH₂)_{β}NR_{g2}R_{g3}, —(CH₂CH₂O)_{α}—(CH₂)_{β}CONR_{g2}R_{g3}, —(CH₂CH₂O)_{α}—(CH₂)_{β}CN, —(CH₂CH₂O)_{α}—(CH₂)_{β}Cl, —(CH₂CH₂O)_{α}—(CH₂)_{β}Br, —(CH₂CH₂O)_{α}—(CH₂)_{β}I, —(CH₂CH₂O)_{α}—(CH₂)_{β}-Phenyl, aryl groups, fused aromatic rings, vinyl, allyl, 4-styryl, acroyl, methacroyl, acrylonitrile, isocyanate, isothiocyanate, epoxides, strained ring olefins, (—CH₂)_{γ}SiCl₃, (—CH₂)_{γ}Si(OCH₂CH₃)₃, or (—CH₂)_{γ}Si(OCH₃)₃;
R_{g1}, R_{g2}, and R_{g3} independently are selected from a hydrogen atom, a linear or branched alkyl group with up to 25 carbons, or a functional group obtained by reaction with: an amino acid, a polypeptide, adenine, guanine, tyrosine, cytosine, uracil, biotin, ferrocene, ruthenocene, cyanuric chloride, or methacryloyl chloride;
α is an integer from 0 to 10, inclusive;
β and γ independently are integers in a range from 1 to 25, inclusive;
and wherein the at least one photocleavable group is bonded to the chromophore moiety through at least one of Rₐ, R_{b}, R_{c}, R_{d}, Rₑ, R_{f}, R_{g}, Rₕ, Rᵢ, Rⱼ, Rₖ, Rₗ, or Rₘ;
(b) the at least one photocleavable group has the formula: 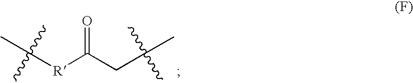
wherein R′ is a substituted or unsubstituted aryl or heteroaryl moiety, wherein any substituents on R′ are selected from alkyl, alkenyl, alkoxy, alkylcarboxy, hydroxy, or carboxy groups, wherein the at least one photocleavable group is bonded to the chromophore moiety through R′ or a substituent on R′, and wherein any substituent on R′ bonded to the chromophore moiety is selected from alkyl, alkenyl, alkoxy, or hydroxy groups; and
(c) the at least one protected group is selected from: 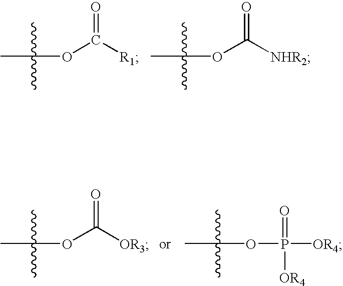
wherein R₁, R₂, R₃, and R₄ independently are selected from a substituted or unsubstituted alkyl, alkenyl, alkynyl, aryl, aralkyl, aralkenyl, aralkynyl, fused aryl, carbocyclic, carboxylalkyl-substituted alkyl, heterocycloalkyl, heterocycloalkyl-substituted alkyl, heteroaryl, heteroarylalkyl, heteroarylalkenyl, or heteroaralkynyl groups, wherein any substituents on R₁, R₂, R₃, and R₄ independently are selected from alkyl, alkenyl, alkynyl, alkoxy, acyl (alkanoyl), acyloxy, cyano, alkylcarboxy, chloro, bromo, aryl, cycloalkyl, aralkyl, aralkenyl, aralkynyl, hydroxy, nitro, amino, carboxy, amino acid, peptide, adenine, guanine, tyrosine, cytosine, uracil, biotin, ferrocene, ruthenocene, cyanuric chloride, methacryloyl chloride, glucose, mannose, galactose, gulose, allose, altrose, idose, talose, fructose, arabinose, xylose, sucrose, cellobiose, maltose, lactose, trehalose, gentiobiose, melibiose, raffinose, gentianose, adenosine, deoxyadenosine, guanosine, deoxyguanosine, cytidine, deoxycytidine, uridine, or deoxythymidine; and
wherein the rate constant for cleavage, k_{cleav}, of a charge-transfer state of the photocleavable group is greater than 0.1 times the rate constant for back-electron transfer, k_{BET}, of the charge-transfer state of the photocleavable group.

18. The compound of claim 17, wherein k_{cleav} is greater than 0.5 times k_{BET}.

19. The compound of claim 17, wherein k_{cleav} is greater than k_{BET}.

20. The compound of claim 17, wherein k_{cleav} is greater than 5 times k_{BET}.

21. The compound of claim 17, wherein the chromophore moiety has a two-photon absorption cross-section of greater than 50×10⁻⁵⁰ cm⁴s/photon.

22. The compound of claim 17, wherein the chromophore moiety has a two-photon absorption cross-section of greater than 100×10⁻⁵⁰ cm⁴s/photon.

23. The compound of claim 17, wherein the chromophore moiety has a two-photon absorption cross-section of greater than 500×10⁻⁵⁰ cm⁴s/photon.

24. The compound of claim 17, wherein the Gibbs free energy change associated with electron transfer from the chromophore moiety to the photocleavable group (ΔG_{ET}) is less than +28 kJ/mol.

25. The compound of claim 17, wherein the Gibbs free energy change associated with electron transfer from the chromophore moiety to the photocleavable group (ΔG_{ET}) is less than +14 kJ/mol.

26. The compound of claim 17, wherein the Gibbs free energy change associated with electron transfer from the chromophore moiety to the photocleavable group (ΔG_{ET}) is less than zero.

27. The compound of claim 17, wherein the rate constant for electron transfer, k_{ET}, of an excited state of the chromophore moiety is greater than 0.1×(rate constant for radiative decay plus the rate constant for non-radiative decay) (k_{rad}+k_{non-rad}) of the excited state of the chromophore moiety.

28. The compound of claim 17, wherein the rate constant for electron transfer, k_{ET}, of an excited state of the chromophore moiety is greater than 0.5×(rate constant for radiative decay plus the rate constant for non-radiative decay) (k_{rad}+k_{non-rad}) of the excited state of the chromophore moiety.

29. The compound of claim 17, wherein the rate constant for electron transfer, k_{ET}, of an excited state of the chromophore moiety is greater than the rate constant for radiative decay plus the rate constant for non-radiative decay (k_{rad}+k_{non-rad}) of the excited state of the chromophore moiety.

30. The compound of claim 17, wherein the compound is:

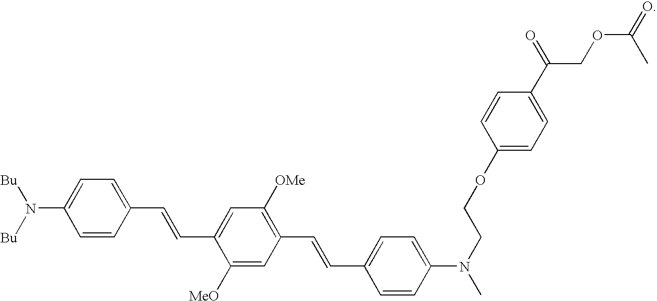

31. A method for deprotecting a protected functional group comprising:
(a) providing a compound according to claim 17;
(b) exposing the compound to radiation;
(c) converting the compound to a multi-photon electronically excited state upon simultaneous absorption of at least two photons of the radiation by the compound, wherein the sum of the energies of all of the absorbed photons is greater than or equal to the transition energy from a ground state of the compound to the multi-photon excited state and wherein the energy of each absorbed photon is less than the transition energy between the ground state and the lowest single-photon excited state of the compound and is less than the transition energy between the multi-photon excited state and the ground state;
thereby inducing an electron transfer from the chromophore moiety to the photocleavable group to cleave and deprotect the protected functional group.

32. A compound selected from: 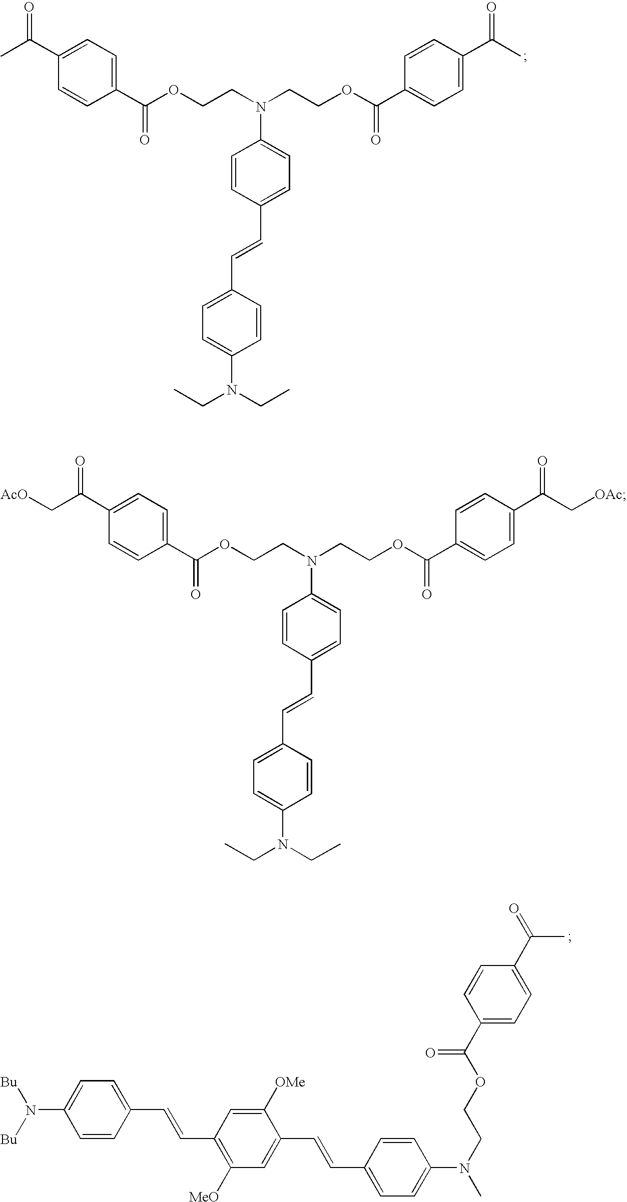 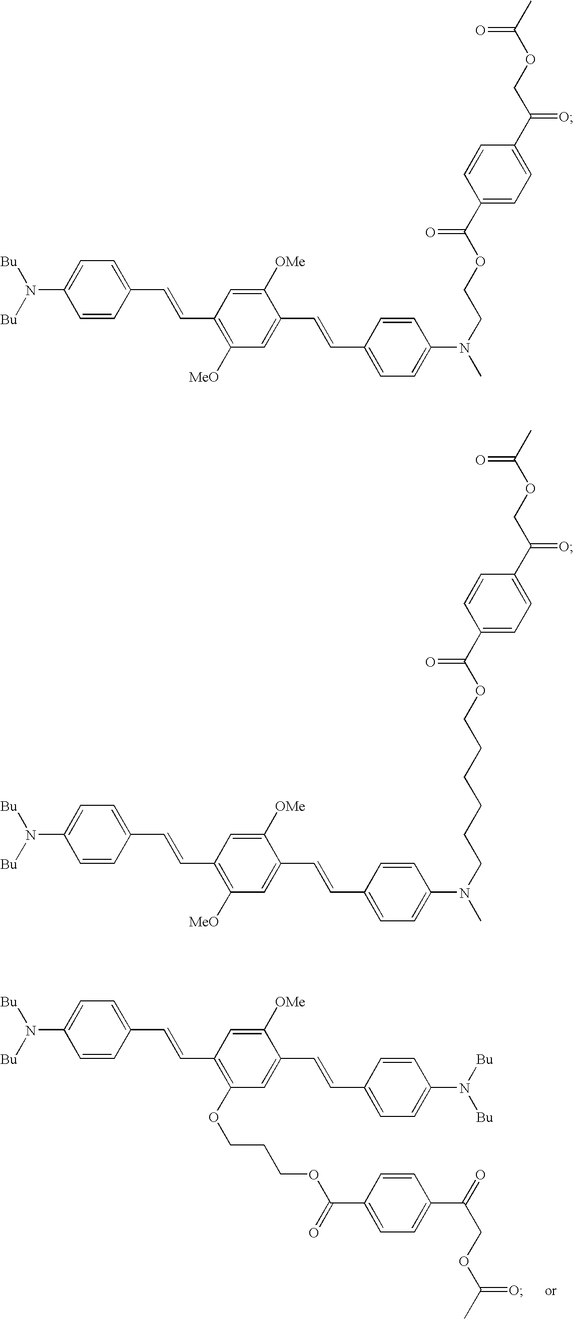 [chemical structure]
